(19) United States
(12) Patent Application Publication
(10) Pub. No.: US 2004/0018561 A1
(43) Pub. Date: Jan. 29, 2004

(54) Peptide compounds and their use as protease substrates

(76) Inventors: Gary A. DeCrescenzo, St. Charles, MO (US); Carol P. Howard, Fenton, MO (US); Joseph G. Rico, Ballwin, MO (US); Kun Zhang, Chesterfield, MO (US)

Correspondence Address: HARNESS, DICKEY, & PIERCE, P.L.C, 7700 BONHOMME, STE 400, ST. LOUIS, MO, 63105, US

(21) Appl. No.: 10/435,486

(22) Filed: May 10, 2003

Related U.S. Application Data
(60) Provisional application No. 60/379,598, filed on May 10, 2002.

(51) Int. Cl. G01N 33/53; C07K 14/00; C07K 7/08

(52) U.S. Cl. 435/7.1; 530/324; 530/402

(57) ABSTRACT

This invention is directed generally to peptide compounds and salts, and particularly to peptide compounds and salts that are useful as protease substrates, such as matrix metalloprotease (“MMP”) substrates. This invention also is directed to methods for making such compounds and salts, as well as amino acids that may, for example, be used in such methods. This invention is further directed to methods for using such compounds and salts to, for example, evaluate the effectiveness of potential protease inhibitors and to detect or monitor a disease associated with protease activity.

### PRIORITY CLAIM TO RELATED PATENT APPLICATIONS

This patent claims priority to U.S. Provisional Patent Application Serial No. 60/379,598 (filed May 10, 2002). The entire text of that application is incorporated by reference into this patent.

### FIELD OF THE INVENTION

This invention is directed generally to peptide compounds and salts, and particularly to peptide compounds and salts that are useful as protease substrates, such as matrix metalloprotease (“MMP”) substrates. This invention also is directed to methods for making such compounds and salts, as well as amino acids that may, for example, be used in such methods. This invention is further directed to methods for using such compounds and salts to, for example, evaluate the effectiveness of potential protease inhibitors and to detect or monitor a disease associated with protease activity.

### BACKGROUND OF THE INVENTION

Matrix metalloproteinases, a family of zinc-dependent proteinases, make up a major class of enzymes involved in degrading connective tissue. Matrix metalloproteinases are divided into several classes, with some members having multiple names in common use. Matrix metalloproteinases include: MMP-1 (also known as collagenase 1, fibroblast collagenase, or EC 3.4.24.3), MMP-2 (also known as gelatinase A, 72 kDa gelatinase, basement membrane collagenase, or EC 3.4.24.24), MMP-3 (also known as stromelysin 1 or EC 3.4.24.17), proteoglycanase, MMP-7 (also known as matrilysin), MMP-8 (also known as collagenase II, neutrophil collagenase, or EC 3.4.24.34), MMP-9 (also known as gelatinase B, 92 kDa gelatinase, or EC 3.4.24.35), MMP-10 (also known as stromelysin 2 or EC 3.4.24.22), MMP-11 (also known as stromelysin 3), MMP-12 (also known as metalloelastase, human macrophage elastase or HME), MMP-13 (also known as collagenase 111), MMP-14 (also known as MT1-MMP or membrane MMP), and MMP-26. See, generally, Woessner, J. F., “The Matrix Metalloprotease Family” in Matrix Metalloproteinases, pp. 1-14 (Edited by Parks, W. C. & Mecham, R. P., Academic Press, San Diego, Calif. 1998). See also, Marchenko, G. N., et al., “Characterization of matrix metalloproteinase-26, a novel metalloproteinase widely expressed in cancer cells of epithelial origin,” Biochem. J., 356:705-718 (2001).

Each MMP comprises a specific amino acid sequence, exhibits a specific cell and tissue distribution, and hydrolytically cleaves a specific subset of target substrate proteins. Generally, the normal substrates of MMPs are other extracellular or cell-surface proteins. Depending on the substrate, cleavage by an MMP either inactivates or activates the substrate (if the substrate is an inactive protein precursor). Because MMPs can activate and inactivate other proteins, MMPs often play key roles in regulation of extracellular signaling, extracellular matrix remodeling, and metabolism. Proper regulation of MMP activity is thus critical to normal development and maintenance of cells and tissues.

Each MMP substrate comprises at least one specific amino acid sequence that the MMP recognizes and binds (the “recognition site”). Associated with each such recognition site is a “scissile bond.” This is a bond that is cleaved by the MMP. Cleavage of the scissile bond results in the formation of two cleavage products. For example, the MMP collagenase cleaves the protein collagen at a single peptide bond at a specific glycine-leucine or glycine-isoleucine sequence. See, e.g., Weingarten, H., et al., “Synthetic Substrates of Vertebrate Collagenase,” Biochemistry, 24:6730-6734 (1985). Specific recognition site sequences are often found in more than one extracellular peptide. Therefore, one MMP may cleave multiple extracellular peptide substrates.

MMP activity can become misregulated. Excessive breakdown of connective tissue by MMPs is a feature of many pathological conditions. Such pathological conditions generally include, for example, tissue destruction, fibrotic diseases, pathological matrix weakening, defective injury repair, cardiovascular diseases, pulmonary diseases, kidney diseases, liver diseases, and diseases of the central nervous system. Specific examples of such conditions include, for example, rheumatoid arthritis, osteoarthritis, septic arthritis, multiple sclerosis, a decubitis ulcer, corneal ulceration, epidermal ulceration, gastric ulceration, tumor metastasis, tumor invasion, tumor angiogenesis, periodontal disease, liver cirrhosis, fibrotic lung disease, emphysema, otosclerosis, atherosclerosis, proteinuria, coronary thrombosis, dilated cardiomyopathy, congestive heart failure, aortic aneurysm, epidermolysis bullosa, bone disease, Alzheimer's disease, defective injury repair (e.g., weak repairs, adhesions such as post surgical adhesions, and scarring), chronic obstructive pulmonary disease, and post myocardial infarction. MMPs (particularly MMP-9) also have been reported to be associated with pathological conditions related to nitrosative and oxidative stress. See Gu, Zezong, et al., “S-Nitrosylation of Matrix Metalloproteinases: Signaling Pathway to Neuronal Cell Death,” Science, 297:1186-90 (2002).

Because abnormal MMP activity causes, promotes, or is necessary for some disease states, MMP inhibitors can be therapeutically beneficial. When developing such inhibitors, it is useful to be able to accurately measure activity levels of specific MMP's in the blood, serum, and/or tissues, both as diagnostic markers and for monitoring the effectiveness of MMP inhibitors. Such an ability, in turn, allows clinicians and researchers to accurately determine parameters of MMP enzyme kinetics, such as the Kₘ, Vₘₐₓ, and k_{cat}/Kₘ, as well as the Kᵢ values of MMP inhibitor compounds. See, e.g., Lehninger, A. L., Biochemistry, pp. 147-168, Worth Publishers, Inc. (1970).

MMP activity can be measured through the use of labeled peptide substrates. See, e.g., Quesada, A. R., et al., “Evaluation of fluorometric and zymographic methods as activity assays for stromelysins and gelatinases,” Clinical Experimental Metastasis, 15:339-340 (1997). In these assays, a substrate peptide comprising an MMP recognition site, a scissile bond, and a detectable label such as a fluorophore (for example, a fluorescein or a coumarin) is mixed with a sample suspected of containing an MMP. Following incubation of the sample with the fluorescently labeled substrate, the mixture is analyzed for the presence and quantity of a fluorescent proteolytic degradation product. Sample analysis can be by any quantifiable method that separates the cleavage product from the uncleaved substrate, such as gel electrophoresis or high pressure liquid chromatography (HPLC). While useful, these assays are limited by the requirement to separate uncleaved substrate from reaction product. In addition, quantification of activity levels over a wide range of substrate concentrations (generally required for accurate determination of enzyme kinetics parameters, such as Kₘ, Vₘₐₓ, and k_{cat}/Kₘ of matrix metalloproteases, as well as inhibitor Kᵢ) can be difficult or impossible because of the limited solubility of many metalloprotease substrate peptides.

An alternative method for detecting and measuring MMP activity is to contact a test sample with a substrate that generates or enhances a fluorescence signal upon hydrolysis by an MMP. For example, a sample can be contacted with a synthetic peptide comprising a tryptophan residue (which is intrinsically fluorescent), an MMP cleavage site, and a quencher for the tryptophan fluorescence, wherein the tryptophan and its quencher are situated on opposite sides of a cleavage site. See, e.g., Stack, M. S., et al., “Comparison of vertebrate collagenase and gelatinase using a new fluorogenic substrate peptide,” The Journal of Biological Chemistry 264:4277-4281 (1989). See, also, Netzel-Arnett, S., et al., “Continuously recording fluorescent assays optimized for five human matrix metalloproteinases,” Analytical Biochemistry 195:86-92 (1991). Cleavage of such a peptide by a metalloprotease separates the tryptophan from the fluorescence quencher, thereby yielding a reaction product that is more fluorescent than the uncleaved substrate. MMP activity consequently can be measured by observing an increase in the intrinsic fluorescence of the sample as the peptide is cleaved. The use of peptide substrates comprising quenched tryptophan tends to be disadvantageous because, for example: (1) tryptophan fluorescence is weak (i.e., has poor quantum yield), (2) the presence of tryptophan in other peptides in a sample interferes with the measurement of enzyme activity, and (3) the use of a tryptophan within a peptide constrains design of synthetic peptides.

A more sensitive alternative to hydrolysis of a quenched tryptophan peptide is hydrolysis of a substrate peptide comprising a highly fluorescent fluorophore and a quencher for the fluorophore, wherein the fluorophore and its quencher are situated on opposite sides of a scissile bond. See, e.g., Knight, C. G., et al., “Fluorometric assays of proteolytic enzymes,” Methods in Enzymology, 248:18-34 (1995). Such a peptide is poorly fluorescent because of the presence of the quencher within the same molecule as the fluorophore. But upon hydrolysis of the peptide at the scissile bond, the fluorophore regains its high fluorescence (i.e., high quantum yield) because the fluorescence quencher is no longer a component of the same molecule. Thus, MMP activity can be measured by monitoring an increase in fluorescence in a sample contacted with a fluorogenic substrate. The use of a fluorogenic peptide substrate provides the advantage of high sensitivity; and obviates the need to separate substrate from cleavage product. This simplifies the task of measuring MMP activity. See, e.g., Knight, C. G., et al., “A novel coumarin-labeled peptide for sensitive continuous assays of the matrix metalloproteinases,” FEBS Lett., 296:263-266 (1992).

Yet another alternative for measuring MMP activity is to use a substrate peptide comprising a fluorophore or a chromophore and a ligand for attaching the substrate to a solid surface. Here, the fluorophore or chromophore are situated on the opposite side of a scissile bond from the ligand for attaching the substrate. When such substrates are attached to a solid surface, they become immobilized and are therefore not in solution. Upon cleavage of the substrate by an MMP, a fluorescent or colored cleavage product is released into solution. The concentration of the released cleavage product is thereby easily measured using standard spectrophotometric or fluorescent techniques.

Many available fluorogenic substrates lack solubility adequate to enable an investigator to measure MMP activity over a sufficiently wide range of substrate concentrations for making accurate measurements of Kₘ, Vₘₐₓ, and k_{cat}/Kₘ of matrix metalloproteases, or Kᵢ values of inhibitor compounds of matrix metalloproteases.

A need continues for matrix metalloprotease substrates, particularly substrates that, for example, are selectively cleaved by a specific matrix metalloprotease of interest (or a selected group of matrix metalloproteases of interest), have a solubility that is greater than the Kₘ value of that particular matrix metalloprotease(s), and are stable in the presence of other constitutive enzymes (including other matrix metalloproteases), present in, for example, blood, serum, and/or tissue samples.

### SUMMARY OF THE INVENTION

This invention provides compounds and salts that tend to be selectively cleaved by a specific matrix metalloprotease (or a select group of matrix metalloproteases), have a solubility that is greater than the Km value of the specific matrix metalloprotease(s), and/or are stable in the presence of other constitutive enzymes, present in, for example, blood, serum, and/or tissue samples.

Briefly, this invention is directed, in part, to a compound or a salt thereof, wherein the compound comprises a peptide and corresponds in structure to Formula (I):

aa₍ᵢ₎-X—Y-aa₍ⱼ₎-Z (I).

Here:

aa₍ᵢ₎ comprises a sequence of i amino acids at the N-terminus of the peptide.

aa₍ⱼ₎ comprises a sequence of j amino acids at the C-terminus of the peptide.

i is an integer from 0 to 5.

j is an integer from 1 to 6.

A fluorophore is covalently attached to the peptide on one side of the X—Y bond, and at least one of a fluorescence quencher and a ligand is covalently attached to the peptide on the other side of the X—Y bond.

Z is a hydroxyl group at the C-terminus of the peptide. Alternatively, Z is a protecting group at the C-terminus of the peptide.

In some such embodiments, X comprises an MMP recognition sequence, and Y comprises an amino acid. In these embodiments, X is not Pro-Gln-Gln, Pro-Tyr-Ala, or Pro-Val-Glu.

In other such embodiments, X comprises an MMP recognition sequence, and Y comprises a bond or amino acid. In these embodiments, the peptide comprises a D-amino acid.

This invention also is directed, in part, to a compound or a salt thereof, wherein the compound comprises a peptide that, in turn, comprises an amino acid selected from the group consisting of phenyloxynorleucine and benzyloxynorleucine.

This invention also is directed, in part, to a method for determining the activity of a matrix metalloprotease. This method comprises combining the matrix metalloprotease with a compound or salt described above.

This invention also is directed, in part, to a method for ex-vivo detection or monitoring of a disease associated with a pathological matrix metalloprotease level. This method comprises measuring the cleavage of a compound or salt described above.

This invention also is directed, in part, to a method for determining the activity of a matrix metalloprotease in a biological sample. This method comprises combining the biological sample with a compound or salt described above to form a mixture, and analyzing the mixture for the presence of a reaction product of the compound or salt with the matrix metalloprotease.

This invention also is directed, in part, to a method for measuring inhibitory activity of a prospective inhibitor of a matrix metalloprotease. This method comprises combining the following to form a mixture:

a compound or salt recited described above,

the prospective inhibitor, and

the matrix metalloprotease.

This mixture, in turn, is analyzed for the presence of a reaction product of the compound or salt with the matrix metalloprotease.

This invention also is directed, in part, to a kit for detecting or monitoring a disease associated with pathological activity of a matrix metalloprotease. This kit comprises a compound or salt described above.

This invention also is directed, in part, to a kit for evaluating the effectiveness of a prospective MMP inhibitor. This kit comprises a compound or salt described above.

This invention also is directed, in part, to a compound or salt thereof, wherein the compound corresponds in structure to Formula (II):

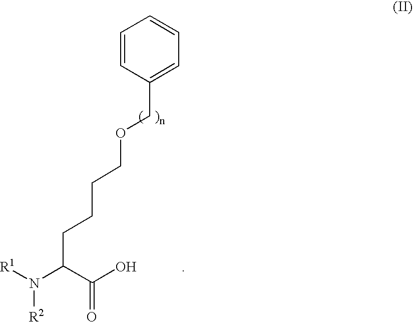

Here:

n is zero or 1.

R¹ and R² are independently selected from the group consisting of hydrogen and a nitrogen protecting group.

Further benefits of Applicants' invention will be apparent to one skilled in the art from reading this patent.

### DETAILED DESCRIPTION OF THE PREFERRED EMBODIMENTS

This detailed description of preferred embodiments is intended only to acquaint others skilled in the art with Applicants' invention, its principles, and its practical application so that others skilled in the art may adapt and apply the invention in its numerous forms, as they may be best suited to the requirements of a particular use. This detailed description and its specific examples, while indicating preferred embodiments of this invention, are intended for purposes of illustration only. This invention, therefore, is not limited to the preferred embodiments described in this patent, and may be variously modified.

A. Compounds of the Invention

The compounds of this invention generally comprise a peptide corresponding in structure to Formula (I):

aa₍ᵢ₎-X—Y-aa₍ⱼ₎-Z (I).

Here, aa₍ᵢ₎ is an amino acid sequence containing i amino acid residues at the N-terminus of the peptide; i is an integer of from zero to about 5; aa₍ⱼ₎ is an amino acid sequence containing j amino acid residues at the C-terminus of the peptide; and j is an integer of from 1 to about 6.

In some preferred embodiments, i is an integer of from zero to 2, and often of from zero to 1.

In some preferred embodiments, j is an integer of from 3 to 6, and often from 3 to 5.

In some preferred embodiments, i is zero, and j is 3. In some other preferred embodiments, i is one, and j is 5.

The amino acid sequences aa₍ᵢ₎ and aa₍ⱼ₎ can contain naturally occurring amino acids or non-naturally occurring synthetic amino acids, including D- and L-amino acids, as well as alpha-, beta-, and gamma-amino acids.

Compounds with D-amino acids and/or other non-naturally occurring amino acids tend to be more resistant to cleavage by constitutive enzymes (e.g., non-specific peptidases) that may be present in biological samples (e.g., blood, serum, tissue, etc.). Compound resistance to cleavage by such enzymes often is an advantage in that it simplifies analysis of cleavage results. Non-naturally occurring amino acids include, for example, N-(imidamidyl)-piperidin-3-yl-L-glycine, 2-aminoadipic acid, 3-aminoadipic acid, beta-alanine, 2-aminobutyric acid, 4-aminobutyric acid, 6-aminocaproic acid, 2-aminoheptanoic acid, 2-aminoisobutyric acid, 2-aminopimelic acid, 2,4-diaminobutyric acid, desmosine, 2,2′-diaminopimelic acid, 2,3-diaminopropionic acid, N-ethylglycine, N-ethylasparagine, hydroxylysine, allo-hydroxylysine, 3-hydroxyproline, 4-hydroxyproline, isodesmosine, allo-isoleucine, N-methylglycine, N-methylisoleucine, 6-N-methyllysine, N-methylvaline, norvaline, norleucine, omithine, 3-(2-napthyl)-L-alanine, O-benzyl-L-tyrosine, 6-(benzyloxy)-L-norleucine, S-(3-phenylpropyl)-L-cysteine, 6-phenoxy-L-norleucine, S-(4-methoxybenzyl)-L-cysteine, and L-mercaptoisocaproic acid (“Mia”). The Examples below illustrate compounds comprising such amino acids.

In some preferred embodiments, aa₍ᵢ₎ and aa₍ⱼ₎ contain alpha-amino acids. In some preferred embodiments, aa₍ᵢ₎ and aa₍ⱼ₎ contain beta- and gamma-amino acids. In some preferred embodiments, aa₍ⱼ₎ contains a sequence of about three D-amino acids at the C-terminus of aa₍ⱼ₎.

The compounds of this invention are preferably sufficiently soluble in water to enable accurate measurement of enzyme activity and parameters of enzyme kinetics (e.g., Kₘ, Vₘₐₓ, and k_{cat}/Kₘ) for specific MMPs, as well as Kᵢ values of prospective inhibitors of specific MMPs. Preferably, the solubility of the compound is at least about 20 μM, more preferably at least about 50 μM, and even more preferably at least about 100 μM in a 1% solution of DMSO in water at pH 7.5.

In some embodiments, at least one amino acid of aa₍ᵢ₎ or aa₍ⱼ₎ contributes to the aqueous solubility of the compound. Preferably more than one amino acid contributes to the compound's aqueous solubility, and more preferably, most (or all) of the aa₍ᵢ₎ and aa₍ⱼ₎ amino acids contribute to the aqueous solubility of the compound.

Amino acids that increase aqueous solubility are generally amino acids that provide a distribution coefficient contribution (log D) of less than about zero. See, generally, Tao et al., “Calculating Partition Coefficients of Peptides by the Addition Method,” Journal of Molecular Modeling 5:189-195 (1999). Log D is the logarithm of the “true” partition coefficient of a compound between octanol and water. Log D accounts for all forms of the compound, including ionized species, and is calculated from the logarithm of the partition coefficient (log P) and the logarithm of the dissociation constant(s) (pKₐ). Amino acids (and their reported Log D contributions) that generally contribute a log D of less than about zero in a peptide include, for example, ornithine (−2.17), arginine (−1.65), aspartic acid (−2.06), glutamic acid (−2.19), histidine (−0.44), asparagine (−0.98), glutamine (−1.00), lysine (−2.27), serine (−0.45), threonine (−0.26), glycine (−0.22), and alanine (−0.27). See Tao, P., et al., J. Mol. Model., 189-195 (1999)). Other non-limiting examples of amino acids that generally contribute a log D of less than about zero in a peptide include beta-alanine, N-(imidamidyl)-piperidin-3-yl-L-alanine, and N-(imidamidyl)-piperidin-3-yl-L-glycine.

In a preferred embodiment, at least one amino acid of aa₍ᵢ₎ and aa₍ⱼ₎ contributes a log D of less than about zero. In some such embodiments, at least one amino acid in each of aa₍ᵢ₎ and aa₍ⱼ₎ contributes a log D of less than about zero. Preferably, aa₍ᵢ₎ and aa₍ⱼ₎ each contain at least one amino acid independently selected from the group consisting of arginine, glutamic acid, aspartic acid, and lysine. In some particularly preferred embodiments, all the amino acids in aa₍ᵢ₎ and aa₍ⱼ₎ contribute a log D of less than about zero.

Z is bound to the carbonyl of the C-terminus of aa₍ⱼ₎. In some embodiments, Z is a hydroxyl group such that the carbonyl group and Z form a carboxy group. In other embodiments, Z is a protecting group. This protecting group preferably confers resistance of the peptide to non-MMP proteases (particularly carboxypeptidases) that may be present in, for example, biological samples (e.g., blood, serum, or tissue). Protecting groups related to peptide chemistry are well known in the art. See generally, Greene, T. W., et al., Protective Groups in Organic Synthesis, 3rd Ed., Wiley: New York (1999) (incorporated by reference into this patent). In some embodiments, for example, Z is an optionally-substituted amino group that is bound to the carbonyl group of the C-terminus forming an amide group—rather than a carboxy group—at the C-terminus. In such instances, Z is —NH₂, —NHR′, and —NHR′R″. Here, R′ and R″ may typically be a wide range of non-hydrogen substituents. In some preferred embodiments, for example, R′ and R″ are independently selected C₁-C₆-alkyl.

X comprises a protease recognition sequence. Typically, a protease recognition sequence is an amino acid sequence that is specifically recognized by a protease of interest. Typically, upon reacting the compound with the suitable protease, the compound's bond that is situated immediately on the carboxy side of the recognition sequence (i.e., the bond between X and Y) is severed (or “cleaved”). This severing typically occurs via a hydrolysis reaction. The bond cleaved by a protease is the scissile bond, and is situated immediately on the carboxy side of X.

In this invention, the protease that recognizes the recognition sequence is typically a matrix metalloprotease. In many embodiments, the recognition sequence is recognized by MMP-2, MMP-9, or MMP-13.

Examples of often suitable MMP recognition sequence include Ala-Gln-Gly, Ala-Met-His, Asn-Gln-Gly, Asp-Lys-Glu, Dnp-Gln-Gly, Dnp-Leu-Gly, Ile-Gly-Phe, Lys-Pro-Asn, Pro-Arg-Gly, Pro-Asp-Gly, Pro-Gln-Tyr, Pro-Gln-Ala, Pro-Gln-Gln, Pro-Gln-Glu, Pro-Gln-Gly, Pro-Gln-His, Pro-Gln-Leu, Pro-Gln-Met, Pro-Gln-Phe, Pro-Gln-Pro, Pro-Gln-Val, Pro-Glu-Asn, Pro-Glu-Gly, Pro-His-Gly, Pro-Leu-Ala, Pro-Leu-Gly, Pro-Met-Gly, Pro-Tyr-Ala, Pro-Tyr-Gly, Pro-Val-Ala, Pro-Val-Glu, Pro-Ser-Glu-Asn, Ser-Gly-Asn, Ser-His-Ser, Ser-Ile-Pro, Thr-Glu-Lys, Tyr-Arg-Trp, Tyr-His-Ser, and Pro-Leu-MeCys. Here, MeCys is S-methyl cysteine.

In some preferred embodiments, the MMP recognition sequence is Ala-Gln-Gly, Ala-Met-His, Asn-Gln-Gly, Asp-Lys-Glu, Dnp-Gln-Gly, Dnp-Leu-Gly, Ile-Gly-Phe, Lys-Pro-Asn, Pro-Arg-Gly, Pro-Asp-Gly, Pro-Gln -Tyr, Pro-Gln-Ala, Pro-Gln-Glu, Pro-Gln-Gly, Pro-Gln-His, Pro-Gln-Leu, Pro-Gln-Met, Pro-Gln-Phe, Pro-Gln-Pro, Pro-Gln-Val, Pro-Glu-Asn, Pro-Glu-Gly, Pro-His-Gly, Pro-Leu-Ala, Pro-Leu-Gly, Pro-Met-Gly, Pro-Tyr-Gly, Pro-Val-Ala, Pro-Ser-Glu-Asn, Ser-Gly-Asn, Ser-His-Ser, Ser-Ile-Pro, Thr-Glu-Lys, Tyr-Arg-Trp, Tyr-His-Ser, or Pro-Leu-MeCys.

In some preferred embodiments, the MMP recognition sequence is Pro-Leu-Gly.

In some preferred embodiments, the MMP recognition sequence is Pro-Gln-Gly.

In some preferred embodiments, the MMP recognition sequence is Pro-Gln-Glu.

In some preferred embodiments, the MMP recognition sequence is Pro-Leu-Glu.

In some preferred embodiments, the MMP recognition sequence is Pro-Leu-MeCys.

In some embodiments, Y is a bond. In this instance, X is bonded directly to aa₍ⱼ₎, and Y is the scissile bond that is cleavable by the particular protease of interest.

In other embodiments, Y comprises a naturally occurring amino acid. Preferred naturally occurring amino acids include, for example, arginine (Arg), glutamine (Gln), glutamic acid (Glu), isoleucine (Ile), leucine (Leu), methionine (Met), phenylalanine (Phe), proline (Pro), serine (Ser), tryptophan (Trp), tyrosine (Tyr), and valine (Val).

In other embodiments, Y comprises a non-naturally occurring amino acid, such as those listed above for aa₍ᵢ₎ and aa₍ⱼ₎. In some preferred embodiments, Y is 3-(2-napthyl)-L-alanine, O-benzyl-L-tyrosine, 6-(benzyloxy)-L-norleucine, S-(3-phenylpropyl)-L-cysteine, 6-phenoxy-L-norleucine, S-(4-methoxybenzyl)-L-cysteine, norvaline (Nva), or L-mercaptoisocaproic acid (Mia).

In some preferred embodiments, Y is a non-naturally occurring amino acid having a side chain comprising at least about 6 (and more preferably at least about eight) non-hydrogen atoms. Such side chains may (and often preferably) comprise one or more bulky structures (e.g., ring structures). However, at least the first atom of the chain (and sometimes more preferably at least the first two atoms of the chain) are linkers that lacking any bulky substituents. Such linkers may be, for example, —C(H)₂—, —O—, —S—, and —N(H)—. In some embodiments, for example, the side chain of Y comprises an alkyl, alkenyl, or alkynyl (preferably an alkyl) substituted with a bulky substituent (e.g., phenyloxy or benzyloxy) at the point on the alkyl, alkenyl, or alkynyl that is furthest from the main peptide chain.

A bulky side chain on the amino acid of Y is often advantageous in contexts where the substrate is used to detect pathological MMP activity in sample containing both MMPs associated pathological activity and other enzymes (including other MMPs) that are more associated with normal bodily functions. For example, MMP-1 and MMP-14, which are typically associated with normal bodily functions, are shallow-pocketed enzymes. In contrast, MMP-2, MMP-9, and MMP-13, which are often associated with pathological conditions, have relatively deep, unobstructed pockets. The pocket depth is dependent on various factors. MMP-1, for example, has an arginine at the P₁′ binding site. This arginine is believed to contribute to MMP-1 having a relatively shallow pocket. Deep-pocketed MMPs, on the other hand, may have, for example, a valine at the same position. A valine is believed to be less obstructive to the pocket. Applicants have discovered that substrates comprising an amino acid with a bulky side chain at the Y position (i.e., the P₁′ position) tend to bind poorly to MMPs that have a shallow pocket (e.g., MMP-1, MMP-7, and MMP-14), while binding relatively well to MMPs that have deep pockets (e.g., MMP-2, MMP-9, and MMP-13). Substrates with such bulky side chains on the amino acid of Y consequently tend to be selectively cleaved by MMPs with deep pockets, and therefore can be advantageously used to detect pathological deep-pocketed MMP activity in biological samples (e.g., blood, serum, and/or tissue), which typically may contain both deep-pocketed and shallow-pocketed MMPs.

In some preferred embodiments, Y is a non-naturally occurring amino acid comprising a side chain of at least 11 non-hydrogen atoms. Examples of side chains include:

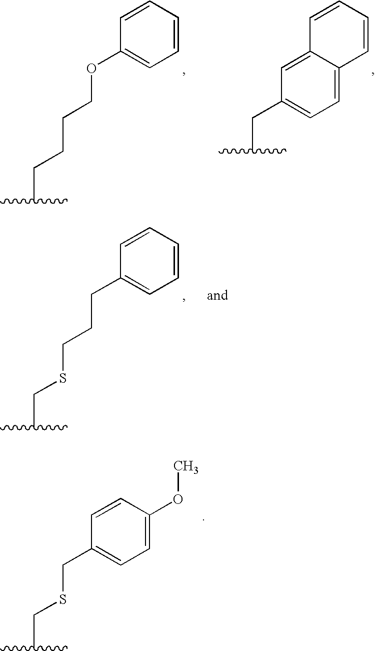

Examples of contemplated compounds comprising a Y having such a side chain include:

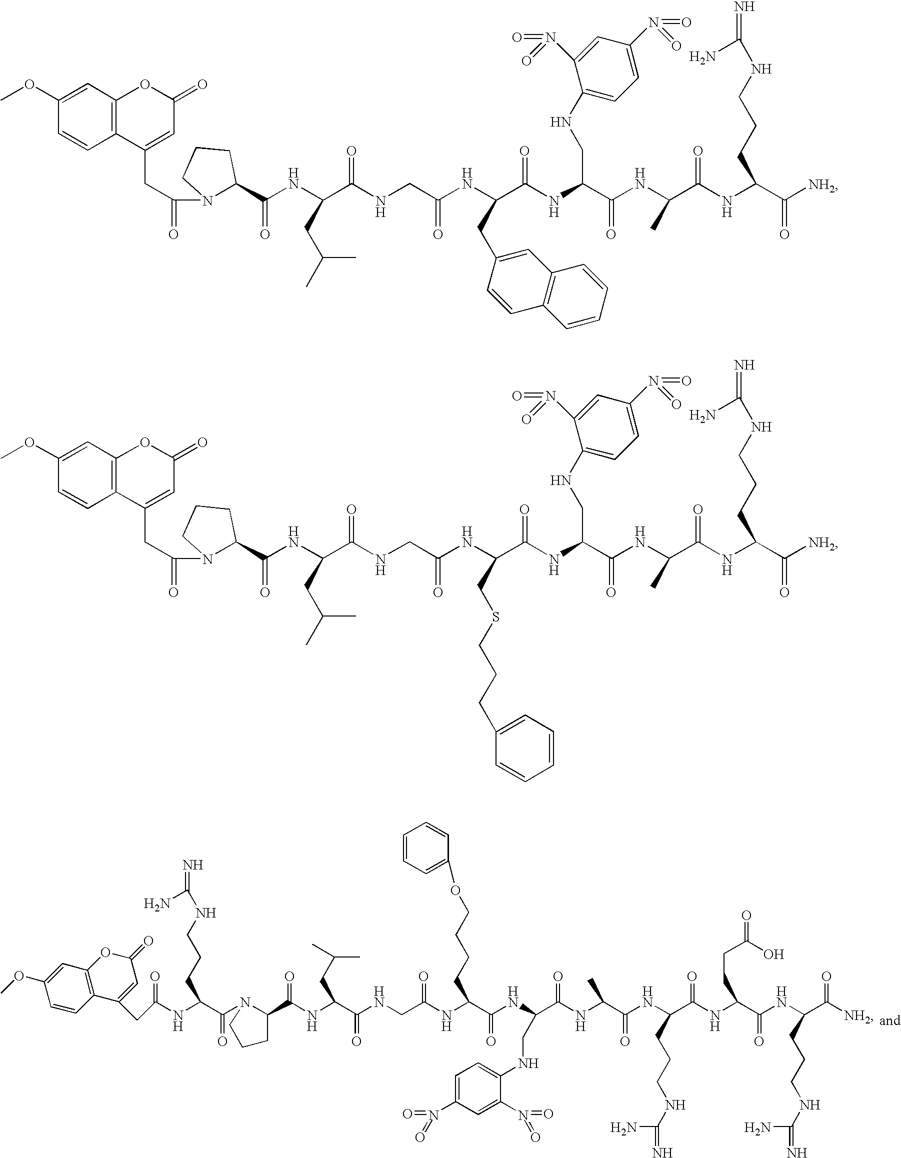

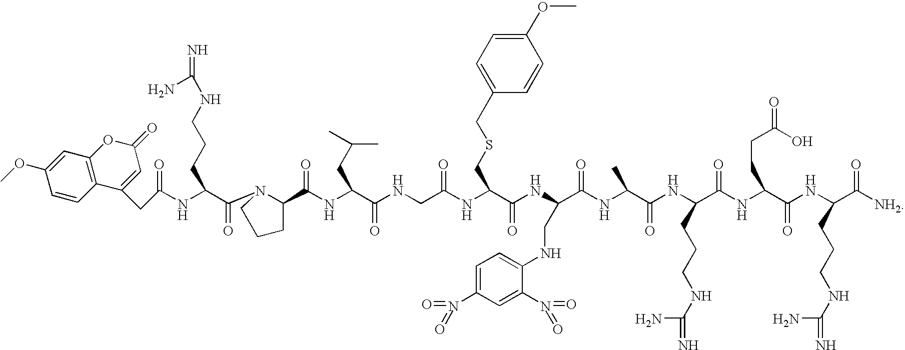

In some preferred embodiments, Y is a non-naturally occurring amino acid comprising a side chain of at least 12 non-hydrogen atoms. Examples of such side chains include:

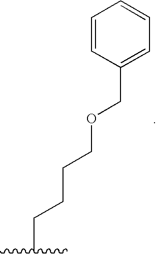

Examples of contemplated compounds comprising a Y having such a side chain include:

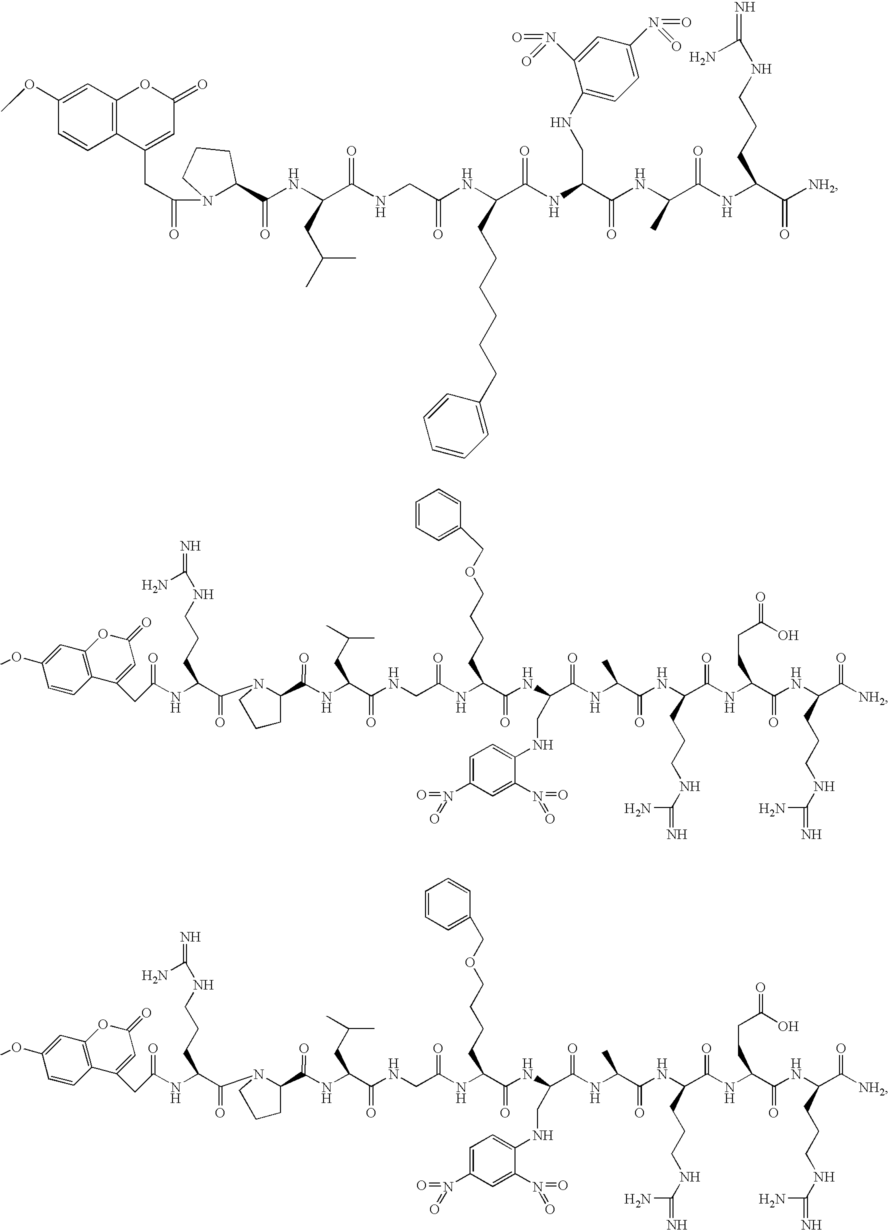

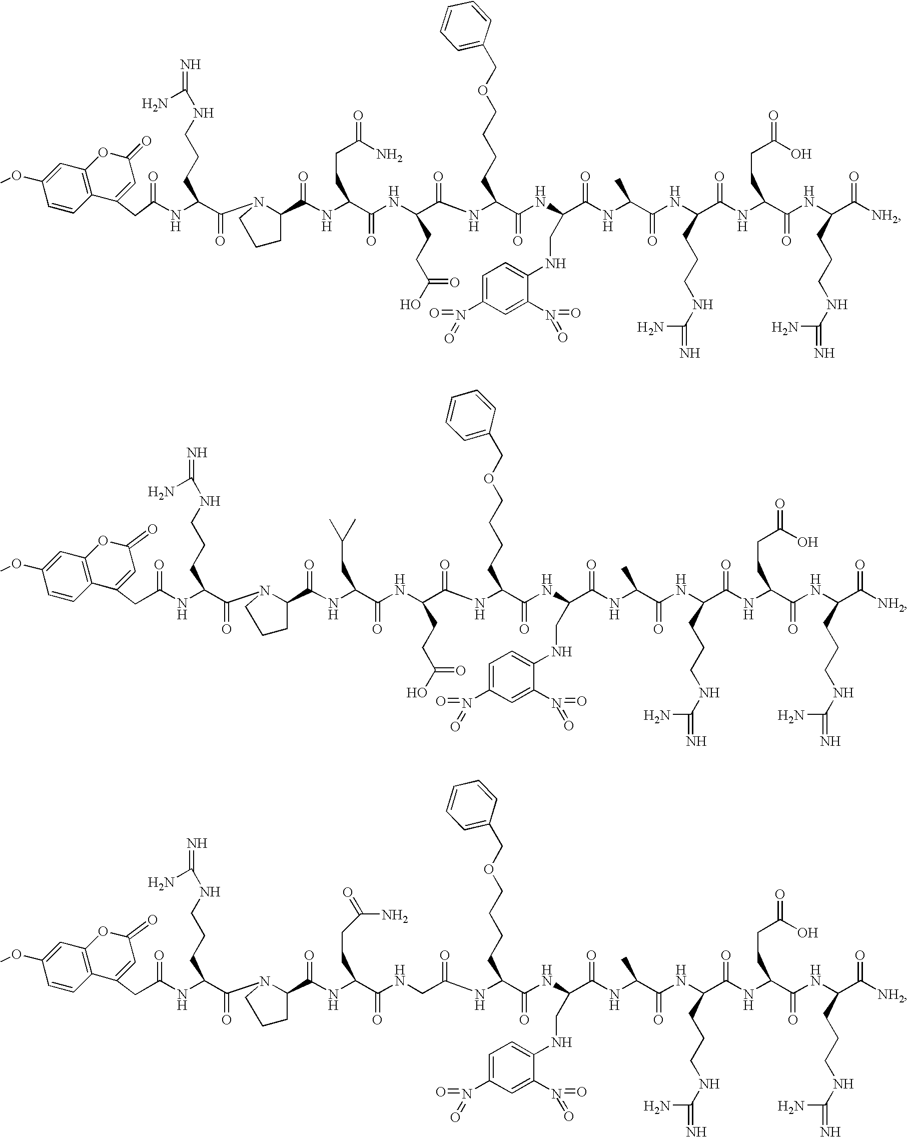

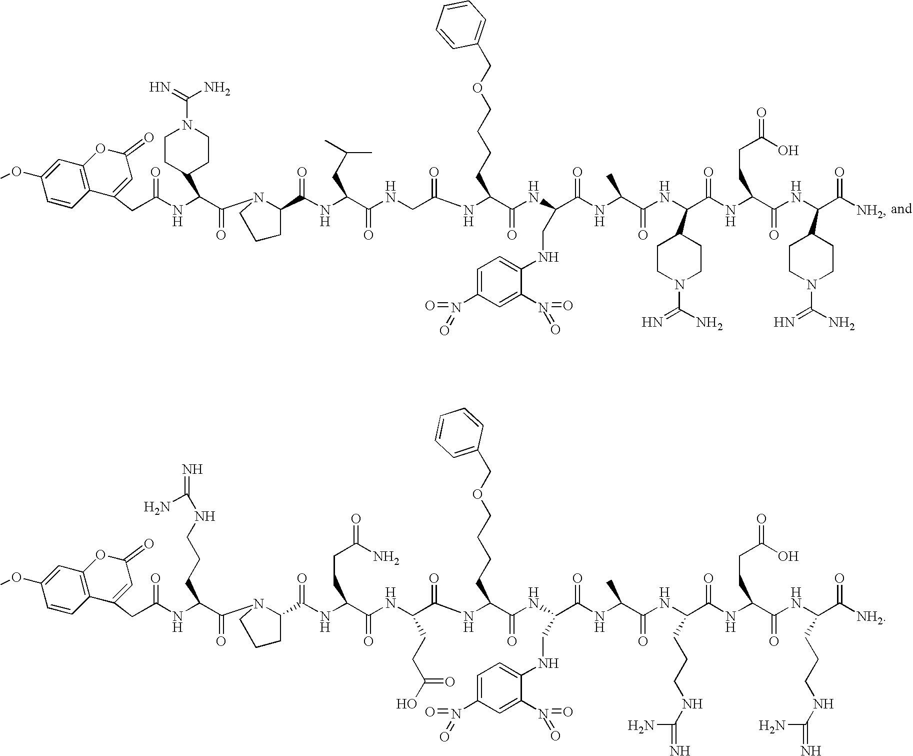

In yet other preferred embodiments, Y is a non-naturally occurring amino acid comprising a side chain of at least 15 non-hydrogen atoms. Examples of such side chains include:

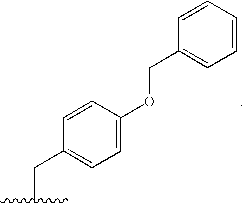

Examples of contemplated compounds comprising a Y having such a side chain include:

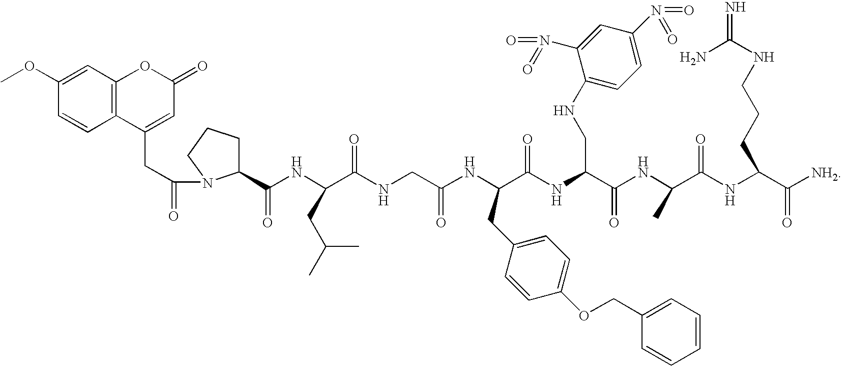

Two examples of amino acids that, when located at P₁′ position, tend to confer often desirable specificity (i.e., that “spares” MMP-1) are 6-(benzyloxy)-norleucine and 6-phenoxy-norleucine. These amino acids correspond in structure to Formula (II):

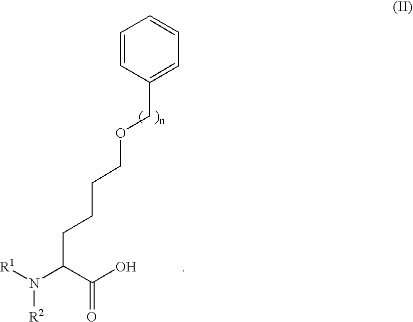

The D- as well as the L-form may be used (but the L-form is often particularly preferred), n is zero or 1, and R¹ and R² are independently selected from the group consisting of hydrogen and a nitrogen protecting group. Nitrogen protecting groups are well known in the art. See generally, Greene, T. W., et al., Protective Groups in Organic Synthesis (3rd Ed., Wiley: New York (1999)),(incorporated by reference into this patent). In some embodiments, R¹ and R² form a 5- to 7-membered ring with the nitrogen. In other embodiments, R¹ is a protecting group (e.g., 9-fluorenylmethoxycarbonyl (“Fmoc”) or t-butyloxycarbonyl (“Boc”)), and R² is hydrogen:

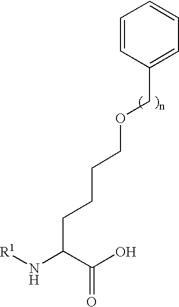

The norleucine may form part of a peptide sequence with or without such an amino protecting group.

Peptide compounds comprising side chains derived from 6-(benzyloxy)-norleucine and 6-phenoxy-norleucine may be synthesized by known solid state or organic synthetic methods. For solid state synthesis, the amino acid may be coupled to an amino protecting group and attached to a growing peptide chain on the resin. Protecting groups common in solid state synthesis include Fmoc and Boc. Examples of synthesis of the Fmoc-protected norleucines are illustrated below in Examples 8 and 25. In a preferred embodiment, a hydroxyl norleucine is first amino-protected, for example by synthesis of the Fmoc derivative. The amino-protected hydroxyl norleucine may then be alkylated with a benzyl or a phenyl group, as exemplified below.

In some preferred embodiments, k_{cat}/Kₘ of the compound with at least one of MMP-1 and MMP-7 is no greater than about 0.5×10⁻⁴ M⁻¹s⁻¹, and the k_{cat}/Kₘ of the compound with at least one of MMP-2, MMP-9, and MMP-13 is at least about 50×10⁻⁴ M⁻¹s¹. In some often more preferred embodiments, the k_{cat}/Kₘ of the compound with at least one of MMP-1 and MMP-7 is no greater than about 10⁻⁵ M⁻¹s⁻¹, and the k_{cat}/Kₘ of the compound with at least one of MMP-2, MMP-9, and MMP-13 is at least about 50×10⁻⁴ M⁻¹s⁻¹.

As may be seen in Table 1 below, compounds comprising 6-(benzyloxy)-L-norleucine (i.e., the compounds of Examples 20 and 21 below) or 6-phenoxy-L-norleucine (i.e., the compound of Example 25 below) exhibited a low k_{cat} or k_{cat} of zero with MMP-1 and MMP-7. It may also be seen from Table 1 that substrates that spare MMP-1 and MMP-7 nevertheless were cleaved by the other MMPs. In Table 1, k_{cat}/Kₘ is given as M⁻¹s⁻¹×10⁴, and Kₘ is μM.

TABLE 1
Kₘ and k_{cat}/Kₘ for Various Substrates with Various Matrix Metalloproteases

| Compound No. | | MMP-1 | MMP-2 | MMP-3 | MMP-7 | MMP-8 | MMP-9 | MMP-13 | MMP-14 |
|---|---|---|---|---|---|---|---|---|---|
| 1 | K_{cat}/Kₘ | 25.5 | 56.5 | 0.98 | 14.2 | 38.1 | 129 | 392 | 35.4 |
| | Kₘ | 16 | 16.5 | 38.7 | 26.4 | 20.1 | 6.2 | 3.9 | 8 |
| 20 | K_{cat}/Kₘ | 0 | 134 | 0.48 | 0 | 17.9 | 147 | 424 | 8.3 |
| | Kₘ | | 8.3 | 85.9 | | 9.1 | 2.3 | 0.9 | 12 |
| 11 | k_{cat}/Kₘ | 17.6 | 68.1 | 0.08 | 12 | 45.6 | 133 | 408 | 30 |
| | Kₘ | 17.3 | 16.3 | 59.5 | 25 | 13.3 | 8.2 | 4.7 | 5.8 |
| 13 | k_{cat}/Kₘ | 5.7 | 17.8 | 0.63 | 2.2 | 13.9 | 41.8 | 29.6 | 4.1 |
| | Kₘ | 49 | 9.9 | 14 | 81.2 | 16.7 | 6.4 | 9 | 27.2 |
| 21 | k_{cat}/Kₘ | 0.36 | 60 | 0.16 | 0.298 | 14.6 | 31.9 | 158 | 5.09 |
| | Kₘ | 35.1 | 3.7 | 28.9 | 94.7 | 3.3 | 3.3 | 1.5 | 23.7 |
| 25 | k_{cat}/Kₘ | 0 | 196 | 0 | 0 | 52.2 | 191 | 293 | 15.5 |
| | Kₘ | | 3.6 | | | 4.4 | 1.4 | 1.2 | 7.4 |

In some preferred embodiments, the compound has a k_{cat} for MMP-2 that is at least about 10 times greater (and more preferably at least about 100 times greater) than its k_{cat} for MMP-1 or MMP-7. In an even more preferred embodiments, the compound has a k_{cat} for MMP-2 that is at least about 10 times greater (and more preferably at least about 100 times greater) than its k_{cat}'s for MMP-1 and MMP-7.

In some preferred embodiments, the compound has a k_{cat} for MMP-9 that is at least about 10 times greater (and more preferably at least about 100 times greater) than its k_{cat} for MMP-1 or MMP-7. In an even more preferred embodiments, the compound has a k_{cat} for MMP-9 that is at least about 10 times greater (and more preferably at least about 100 times greater) than its k_{cat}'s for MMP-1 and MMP-7. Such a selectivity may be particularly useful in embodiments wherein the compound is added to a biological sample (fluid or tissue from an eye) to diagnose or monitor the status of a disease of the eye.

In some preferred embodiments, the compound has a k_{cat} for MMP-13 that is at least about 10 times greater (and more preferably at least about 100 times greater) than its k_{cat} for MMP-1 or MMP-7. In an even more preferred embodiments, the compound has a k_{cat} for MMP-13 that is at least about 10 times greater (and more preferably at least about 100 times greater) than its k_{cat}'s for MMP-1 and MMP-7.

Table 2 below illustrates several sequences that have been reported to be cleaved by matrix metalloproteases. The recognition sequence in those illustrations is denoted as P₃-P₂-P₁. The P₁′ amino acid is the amino acid that is bonded to the recognition sequence via the scissile bond (i.e., the P₁-P₁′ bond is the bond that has reportedly been cleaved by the listed MMP(s)). In some embodiments, the compounds of this invention comprise such cleavage sites. In those embodiments, Formula (I) is defined such that X is P₃-P₂-P₁ in Table 2, Y is the corresponding P₁′ in Table 2, and the amino acid of aa₍ⱼ₎ bonded to Y is the corresponding P₂′ in Table 2.

TABLE 2
Peptide Sequences Known To Be Cleaved By Matrix Metalloproteases

| | | | | | | Reported to be | Reported to be |
|---|---|---|---|---|---|---|---|
| | | | | | | cleaved by the | poorly cleaved by |
| | | | | | | following | the following |
| P₃ | P₂ | P₁ | P₁′ | P₂′ | Ref | MMP(s): | MMP(s): |
| Pro | Val | Ala | Val | Ser | 1 | 26 | |
| Pro | Glu | Gly | Ile | Asp | 1 | 26 | |
| Lys | Pro | Asn | Met | Ile | 1 | 26 | |
| Thr | Glu | Lys | Leu | Val | 1 | 26 | |
| Asp | Lys | Glu | Leu | Arg | 1 | 26 | |
| Pro | Leu | Gly | Leu | Dpa | 1, 3 | 1, 2, 3, 7, (9,13), 14 | 26 |
| Pro | Leu | Ala | Leu | Dpa | 1 | 14 | 26 |
| Pro | Tyr | Ala | Nva | Trp | 1, 2 | 2, 3, 9, 14 | 1, 26 |
| Pro | Val | Ala | Nva | Trp | 1 | | 14, 26 |
| Pro | Val | Glu | Nva | Trp | 2 | 3 | 1, 2, 9 |
| Pro | Leu | Gly | Leu | Trp | 3 | 1, 2, 3, 7 | |
| Pro | Gln | Gln | Phe | Phe | 2, 4, 6 | 1, 2, 3, 9 | |
| Pro | Leu | Ala | Nva | Trp | 4 | 3 | |
| Pro | Leu | Gly | Ile | Ala | 5, 7, 9 | 1, 2, 7, 8, 9 | |
| Dnp | Leu | Gly | Ile | Ala | 5 | | 1 |
| Pro | Gln | Gly | Ile | Ala | 5 | 1, 2, 7, 8, 9 | |
| Dnp | Gln | Gly | Ile | Ala | 5 | | 1 |
| Pro | Gln | Gly | Leu | Ala | 7 | 1, 2, 7, 8, 9, (13) | |
| Pro | Gln | Gly | Leu | Leu | | | |
| Ala | Gln | Gly | Ile | Ala | 7 | 1 | 2, 7, 8, 9 |
| Asn | Gln | Gly | Ile | Ala | 7 | 2 | 1, 7, 8, 9 |
| Pro | Hyp | Gly | Ile | Ala | 7 | | 1, 2, 7, 8, 9 |
| Pro | Arg | Gly | Ile | Ala | 7 | 2, 9 | 1, 7, 8 |
| Pro | Asp | Gly | Ile | Ala | 7 | | 1, 2, 7, 8, 9 |
| Pro | Val | Gly | Ile | Ala | 7 | 2, 9 | 1, 7, 8 |
| Pro | Met | Gly | Ile | Ala | 7 | 1, 2, 7, 8, 9 | |
| Pro | Tyr | Gly | Ile | Ala | 7 | 1, 2, 7, 8, 9 | |
| Pro | Gln | Met | Ile | Ala | 7 | 1, 7, 8 | 2, 9 |
| Pro | Gln | Glu | Ile | Ala | 7 | 7, 8 | 1, 2, 9 |
| Pro | Gln | Tyr | Ile | Ala | 7 | 1, 8 | 2, 7, 9 |
| Pro | Gln | Ala | Ile | Ala | 7 | 1, 2, 7, 8, 9 | |
| Pro | Gln | Pro | Ile | Ala | 7 | 1, 7, 8 | 2, 9 |
| Pro | Gln | Gln | Ile | Ala | 7 | 7, 8 | 1, 2, 9 |
| Pro | Gln | Phe | Ile | Ala | 7 | 1, 7, 8 | 2, 9 |
| Pro | Gln | Leu | Ile | Ala | 7 | 7, 8 | 1, 2, 9 |
| Pro | Gln | Val | Ile | Ala | 7 | | 1, 2, 7, 8, 9 |
| Pro | Gln | His | Ile | Ala | 7 | 1, 2 | 7, 8, 9 |
| Pro | Gln | Gly | Trp | Ala | 7 | | 1, 2, 7, 8, 9 |
| Pro | Gln | Gly | Pro | Ala | 7 | | 1, 2, 7, 8, 9 |
| Pro | Gln | Gly | Glu | Ala | 7 | | 1, 2, 7, 8, 9 |
| Pro | Gln | Gly | Tyr | Ala | 7 | 8, 9 | 1, 2, 7 |
| Pro | Gln | Gly | Phe | Ala | 7 | | 1, 2, 7, 8, 9 |
| Pro | Gln | Gly | Met | Ala | 7 | 1, 2, 7, 8, 9 | |
| Pro | Gln | Gly | Val | Ala | 7 | | 1, 2, 7, 8, 9 |
| Pro | Gln | Gly | Gln | Ala | 7 | | 1, 2, 7, 8, 9 |
| Pro | Gln | Gly | Ser | Ala | 7 | | 1, 2, 7, 8, 9 |
| Pro | Gln | Gly | Arg | Ala | 7 | | 1, 2, 7, 8, 9 |
| Ile | Gly | Phe | Leu | Arg | 8 | 2, 9, 14 | |
| Ala | Met | His | Met | Tyr | 8 | 14 | 2, 9 |
| Ser | Glu | Asn | Ile | Arg | 8 | 14 | 2, 9 |
| Pro | Glu | Asn | Ile | Arg | 8 | 9, 14 | |
| Tyr | Arg | Trp | Leu | Thr | 8 | 2, 9, 14 | |
| Ser | His | Ser | Ile | Thr | 8 | 14 | 2, 9 |
| Ser | Gly | Asn | Leu | Arg | 8 | 2, 14 | 9 |
| Ser | Ile | Pro | Leu | Thr | 8 | 9, 14 | 2 |
| Tyr | His | Ser | Leu | Thr | 8 | 14 | 9 |
| Pro | Leu | Gly | Mia | Leu | 9 | 1 | |
| Pro | Val | Glu | Nva | Ala | 10 | 3, 13 | 1, 2, 8, 9 |
| Pro | Gln | Gly | Leu | Cys(F1) | 10 | 2, 13 | 1, 3, 8, 9 |

In Table 2, all amino acids are L-amino acids unless “D” is specified, Dnp is 2,4-dinitrophenyl, Dpa is 3-[2,4-dinitrophenyl]-L-2,3-diaminopropionic acid, Mia is L-mercaptoisocaproic acid (scissile bond is “thiopeptolide”), Nva is norvaline, and Cys(Fl) is Cys (Fluorescein).

The references in Table 2 are as follows:

1. Marchenko, G. N., et al., “Characterization of matrix metalloproteinase-26, a novel metalloproteinase widely expressed in cancer cells of epithelial origin,” Biochem. J., 356:705-718 (2001).

2. Nagase, H., et al., “Design and Characterization of a Fluorogenic Substrate Selectively Hydrolyzed by Stromelysin 1 (Matrix Metalloproteinase-3),” J. Biol. Chem., 269:20952-7 (1994).

3. Knight, C. G., et al., “A novel coumarin-labeled peptide for sensitive continuous assays of the matrix metalloproteinases,” FEBS Lett., 296:263-266 (1992).

4. Bickett, D. M., et al., “A High Throughput Fluorogenic Substrate for Stromelysin (MMP-3), Ann. New York Acad. Sci., 732:351-355 (1994).

5. Masui, Y., et al., “Synthetic Substrates for Vertebrate Collagenase,” Biochem. Med., 17:215-221 (1977).

6. Teahan, J., et al., “Substrate specificity of human fibroblast stromelysins. Hydrolysis of substance P and its analogues,” Biochem., 28:8497-8501 (1989).

7. Netzel-Arnett, S., et al., “Comparative sequence specificities of human 72- and 92-kDa gelatinases (type IV collagenases) and PUMP (matrilysin),” Biochemistry, 32(25):6427-32 (1993).

8. Kridel, S. J., et al., “A unique substrate binding mode discriminates membrane type 1-matrix metalloproteinase (MT1-MMP) from other matrix metalloproteinases,” J. Biol. Chem., Manuscript in Press M111574200 (pub. Apr. 16, 2002).

9. Weingarten, H., et al., “Synthetic Substrates of Vertebrate Collagenase,” Biochem., 24:6730-6734 (1985).

10. Beekman, B., et al., “Fluorogenic MMP activity assay for plasma including MMPs complexed to α2-macroglobulin,” Ann. New York Acad. Sci., 878:150-158 (1999).

The compounds of the present invention comprise at least one marker moiety. The marker moiety generally comprises a fluorophore. The fluorophore is bonded (typically via a covalent bond) to an amino acid of the compound.

In general, the fluorophore may be naturally-fluorescing moiety. More typically, the fluorophore is chemical group that fluoresces when excited (generally with electromagnetic radiation). Fluorophores often are conjugated double bond systems, most often in ring systems or conjugated ring systems. Fluorophores are well known in the art, and include, for example, coumarins, fluoresceins, rhodamines, Lucifer yellows, and indocyanines. In some particularly preferred embodiments, the fluorophore is a (7-methoxy-2-oxo-2H-chromen-4-yl)acetyl moiety. In some such embodiments, this fluorophore is attached to the N atom of an arginine residue to give N-2-[(7-methoxy-2-oxo-2H-chromen-4-yl)acetyl-L-arginine. Other examples of generally suitable amino acids bonded to fluorophores include N-6-(4{[3-carboxy-4-(6-hydroxy-3-oxo-3H-xanthen-9-yl)phenyl]amino}-6-chloro-1,3,5-triazin-2-yl)-L-lysine, and N-6-(4{[3-carboxy-4-(6-hydroxy-3-oxo-3H-xanthen-9-yl)phenyl]amino}-6-[(2-hydroxyethyl)thio]-1,3,5-triazin-2-yl)-L-lysine.

In some embodiments, the fluorophore is covalently attached to an amino acid of the sequences aa₍ᵢ₎ and aa₍ⱼ₎. In some embodiments, the fluorophore may be covalently attached to the N-terminus amino group of the peptide of Formula (I). In some such embodiments, for example, i is zero, and X comprises a fluorophore at the N-terminus of the protease recognition sequence. In other such embodiments, i is other than zero, and the fluorophore is attached to the N-terminus of aa₍ᵢ₎.

In some preferred embodiments, Y comprises an amino acid covalently bonded to the fluorophore. The compounds of this invention further comprise a fluorescence quencher or a ligand bonded (generally via a covalent bond) to an amino acid that is on the opposite side of the X—Y bond from the amino acid to which the fluorophore is bonded. This results in the fluorophore being on a separate reaction product than the quencher or ligand if the X—Y bond is cleaved by a protease. In some embodiments, for example, the fluorophore is covalently bonded to an amino acid on the amino side of the X—Y bond (i.e., to an amino acid of X or aa₍ᵢ₎), while a ligand or a fluorescence quencher is covalently bonded to the carboxy side of the X—Y bond (i.e., to an amino acid of Y or, often more preferably, aa₍ⱼ₎). In other embodiments, for example, the fluorophore is covalently bonded to an amino acid on the carboxy side of the X—Y bond (i.e., to an amino acid of Y or, often more preferably, aa₍ⱼ₎), while a ligand or a fluorescence quencher is covalently bonded to the amino side of the X—Y bond (i.e., to an amino acid of X or aa₍ᵢ₎).

It should be recognized that this invention contemplates compounds comprising more than one fluorophore. This invention also contemplates compounds comprising more than one fluorescence quencher or ligand opposite the X—Y bond from the fluorophore(s). This invention further contemplates compounds comprising a combination of one or more fluorescence quenchers and one or more ligands on the opposite side opposite the X—Y bond from the fluorophore(s).

Fluorescence quenchers are well known in the art. Generally, quenchers are organic groups that, when positioned in close geometric proximity to a fluorescing group, are capable of providing alternate non-radiative pathways to dissipate the energy held in the excited state of the fluorescer. The result is that fluorescence of the fluorescing group is attenuated or eliminated as long as the quenching molecule or group is in such close proximity. Thus, where the fluorophore and its corresponding quencher are positioned on opposite sides of the scissile bond, cleavage of the scissile bond by a protease causes the fluorophore and the quencher to lose their close proximity, thereby allowing the fluorophore to fluoresce. Thus, compound cleavage may be detected by measuring the change in fluorescence.

In some preferred embodiments wherein the compound comprises a quencher, the quencher comprises a dinitrophenyl group. Such groups are conveniently incorporated into the peptides of the invention by covalently attaching them to the side chain of an amino acid. Fluorescence quencher groups may be incorporated into the substrate peptides by first attaching them to an amino acid, followed by incorporation of the amino acid into the peptide. Alternatively, the quenching group may be covalently attached to an amino acid of a peptide after the peptide is formed. Examples of suitable an amino acid comprising a dinitrophenyl group include 3-[(2,4-dinitrophenyl)amino]-L-alanine.

In embodiments wherein the compounds of this invention comprise a ligand, the ligand is typically capable of binding to a solid support. The form of the solid support may vary widely. The solid support may, for example, be a film, beads, nanoparticles, or an assay plate derivatized with a binding partner of the ligand. The use of ligands and solid supports is well known in the art.

In many embodiments where the compound comprises a ligand, the ligand and fluorophore are on opposite sides of the scissile bond. Thus, cleavage of the scissile bond by a protease produces a cleavage product containing the fluorophore. This cleavage product is free to go into solution. Measurement of the increase in solution fluorescence can then be used to detect cleavage of the compound. In these embodiments, a quencher may optionally be present on the same side of the compound as the ligand.

In other embodiments where the compound comprises a ligand, the ligand and fluorophore are the same side as the scissile bond, and a quencher is on the opposite side of the scissile bond. Here, cleavage of the scissile bond by a protease produces a cleavage product containing the quencher. This quencher therefore is free to go into solution, thereby allowing the fluorophore to fluoresce. Measurement of the increase in fluorescence on the support can then be used to detect cleavage of the compound.

In some embodiments, the ligand comprises a biotin moiety. Examples of such ligands include N-2-[(5-(2-oxohexahydro-1H-thieno[3,4-d]imidazol-4-yl)pentanoyl] or iminobiotin. In such instances, the binding partner (i.e., the binding component on the surface of the solid support) can be, for example, avidin, streptavidin, monomeric avidin, or an anti-biotin antibody.

Other ligands for which a binding partner is available may alternatively (or additionally) be used. Examples of ligands and their binding partners include haptens and anti-hapten antibodies such as digoxygenin and anti-digoxygenin, polyhistidine and immobilized nickel ions, and FLAG sequences and anti-FLAG antibodies.

In some embodiments, the ligand comprises a reactive group for covalent attachment of the peptide to a solid support. In some such embodiments, for example, the ligand contains an amino group. In some preferred embodiments, the ligand comprises a primary amino group. In some such embodiments, for example, the ligand comprises an epsilon-amino caproic acid group.

In many embodiments of this invention, one of aa₍ᵢ₎ and aa₍ⱼ₎ comprises an amino acid covalently attached to a fluorophore, while the other of aa₍ᵢ₎ and aa₍ⱼ₎ comprises an amino acid covalently attached to either a ligand capable of binding to a solid support or a fluorescence quencher of the fluorophore. Here, the fluorophore is on the opposite side of the scissile bond (i.e., the X—Y bond) from a quencher or a ligand. Examples 2-5 below illustrate such configurations.

In some such embodiments, one of aa₍ᵢ₎ and aa₍ⱼ₎ comprises an amino acid covalently linked to a fluorophore, while the other of aa₍ᵢ₎ and aa₍ⱼ₎ comprises (1) an amino acid covalently attached to a fluorescence quencher, and (2) an amino acid covalently attached to a ligand capable of binding to a solid support. Here, the fluorophore is on the opposite side of the scissile bond (i.e., the X—Y bond) from a quencher and a ligand.

This invention also contemplates compounds wherein one of aa₍ᵢ₎ and aa₍ⱼ₎ comprises an amino acid covalently attached to a quencher, while the other of aa₍ᵢ₎ and aa₍ⱼ₎ comprises (1) an amino acid linked to a fluorophore, and (2) an amino acid linked to a ligand capable of binding to a solid support. Here, the fluorophore and the ligand are on the opposite side of the scissile bond (i.e., the X—Y bond) from the quencher. Example 16 below illustrates such a configuration. Typically, in such a configuration, there is no ligand present on the side of the compound comprising the quencher.

In some embodiments, a ligand, fluorophore, or fluorescence quencher is attached to the recognition sequence X. Such embodiments include instances wherein i is zero. In those instances, the ligand, fluorophore, or fluorescence quencher preferably is attached to the N-terminus of the recognition sequence X. Examples 6-9 below illustrate such a configuration. In those illustrations, a fluorophore is covalently attached to the N-terminus of X, and a quencher is covalently attached to the amino acid sequence aa₍ⱼ₎.

The compounds listed in Table 1 and other non-limiting examples of substrates according to the invention are listed in Table 3 (SEQ ID NO. 1 through SEQ ID NO. 29), and in Examples 1-29 below. In Table 3, the structures are written as peptides, with the naturally occurring amino acids given their conventional three letter abbreviation. Non-naturally occurring amino acids, including the D-version of the naturally occurring ones, are indicated in the structure listing as Xaaₙ and further defined in the columns at right of Table 3 and in the footnotes.

TABLE 3

| | | | | | | | | | SEQ |
|---|---|---|---|---|---|---|---|---|---|
| Compound | Structure | R | Xaa₁ | Xaa₂ | Xaa₃ | Xaa₄ | Xaa₅ | Xaa₆ | ID |
| 1 | R-Arg-Pro-Leu-Gly-Leu-Xaa₁-Ala-Arg-Glu-Arg-NH₂ | R₁ | R₃ | — | — | — | — | — | 1 |
| 2 | R-Arg-Pro-Leu-Gly-Leu-Xaa₁-Ala-Arg-Glu-Arg-NH₂ | R₂ | R₄ | — | — | — | — | — | 2 |
| 3 | R-Arg-Pro-Leu-Gly-Leu-Xaa₁-Ala-Arg-Glu-Arg-NH₂ | R₂ | R₅ | — | — | — | — | — | 3 |
| 4 | R-Glu-Pro-Leu-Gly-Leu-Xaa₁-Ala-Glu-Arg-Glu-NH₂ | R₂ | R₄ | — | — | — | — | — | 4 |
| 5 | R-Glu-Pro-Leu-Gly-Leu-Xaa₁-Ala-Glu-Arg-Glu-NH₂ | R₂ | R₅ | — | — | — | — | — | 5 |
| 6 | R-Pro-Leu-Gly-Xaa₁-Xaa₂-Ala-Arg-NH₂ | R₁ | R₄ | R₃ | — | — | — | — | 6 |
| 7 | R-Pro-Leu-Gly-Xaa₁-Xaa₂-Ala-Arg-NH₂ | R₁ | R₇ | R₃ | — | — | — | — | 7 |
| 8 | R-Pro-Leu-Gly-Xaa₁-Xaa₂-Ala-Arg-NH₂ | R₁ | R₈ | R₃ | — | — | — | — | 8 |
| 9 | R-Pro-Leu-Gly-Xaa₁-Xaa₂-Ala-Arg-NH₂ | R₁ | R₉ | R₃ | — | — | — | — | 9 |
| 10 | R-Arg-Pro-Leu-Gly-Leu-Xaa₁-Ala-Arg-Glu-Xaa₂-NH₂ | R₁ | R₃ | D-Arg | — | — | — | — | 10 |
| 11 | R-Arg-Pro-Leu-Gly-Leu-Xaa₁-Ala-Xaa₂-Xaa₃-Xaa₄-NH₂ | R₁ | R₃ | D-Arg | D-Glu | D-Arg | — | — | 11 |
| 12 | R-Xaa₁Pro-Leu-Gly-Leu-Xaa₂-Ala-Arg-Xaa₃-Xaa₄-NH₂ | R₁ | D-Arg | R₃ | D-Glu | D-Arg | — | — | 12 |
| 13 | R-Xaa₁-Pro-Leu-Gly-Leu-Xaa₂-Ala-Xaa₃-Xaa₄-Xaa₅-NH₂ | R₁ | D-Arg | R₃ | D-Arg | D-Glu | D-Arg | — | 13 |
| 14 | R-Xaa₁-Pro-Gln-Gly-Leu-Xaa₂-Ala-Xaa₃-Xaa₄-Xaa₅-NH₂ | R₁ | D-Arg | R₃ | D-Arg | D-Glu | D-Arg | — | 14 |
| 15 | R-Xaa₁-Pro-Leu-Gly-Leu-Xaa₂-Ala-Xaa₃-Xaa₄-Xaa₅-NH₂ | R₁ | D-Lys | R₃ | D-Arg | D-Glu | D-Arg | — | 15 |
| 16 | R-Xaa₁-Pro-Leu-Gly-Leu-Xaa₂-Ala-Xaa₃-Xaa₄-Xaa₅-NH₂ | R₁ | R₁₃ | D-Arg | D-Glu | D-Arg | | 16 | |
| 17 | R-Xaa₁-Pro-Leu-Gly-Leu-Xaa₂-Xaa₃-Xaa₄-Xaa₅-Xaa₆-NH₂ | R₁ | D-Arg | R₃ | β-Ala | D-Arg | β-Ala | D-Arg | 17 |
| 18 | R-Xaa₁-Pro-Leu-Xaa₂-Leu-Xaa₃-Ala-Xaa₄-Xaa₅-Xaa₆-NH₂ | R₁ | D-Arg | R₁₄ | R₃ | D-Arg | D-Glu | D-Arg | 18 |
| 19 | R-Xaa₁-Pro-Leu-Gly-Leu-Xaa₂-Ala-Xaa₃-Glu-Xaa₄-NH₂ | R₁ | R₁₂ | R₃ | R₁₂ | R₁₂ | — | — | 19 |
| 20 | R-Arg-Pro-Leu-Gly-Xaa₁-Xaa₂-Ala-Arg-Glu-Arg-NH₂ | R₁ | R₈ | R₃ | — | — | — | — | 20 |
| 21 | R-Xaa₁-Pro-Leu-Gly-Xaa₂-Xaa₃-Ala-Xaa₄-Xaa₅-Xaa₆-NH₂ | R₁ | D-Arg | R₈ | R₃ | D-Arg | D-Glu | D-Arg | 21 |
| 22 | R-Xaa₁-Pro-Gln-Glu-Xaa₂-Xaa₃-Ala-Xaa₄-Xaa₅-Xaa₆-NH₂ | R₁ | D-Arg | R₈ | R₃ | D-Arg | D-Glu | D-Arg | 22 |
| 23 | R-Xaa₁-Pro-Leu-Glu-Xaa₂-Xaa₃-Ala-Xaa₄-Xaa₅-Xaa₆-NH₂ | R₁ | D-Arg | R₈ | R₃ | D-Arg | D-Glu | D-Arg | 23 |
| 24 | R-Xaa₁-Pro-Gln-Gly-Xaa₂-Xaa₃-Ala-Xaa₄-Xaa₅-Xaa₆-NH₂ | R₁ | D-Arg | R₈ | R₃ | D-Arg | D-Glu | D-Arg | 24 |
| 25 | R-Xaa₁-Pro-Leu-Gly-Xaa₂-Xaa₃-Ala-Xaa₄-Xaa₅-Xaa₆-NH₂ | R₁ | D-Arg | R₁₀ | R₃ | D-Arg | D-Glu | D-Arg | 25 |
| 26 | R-Xaa₁-Pro-Leu-Gly-Xaa₂-Xaa₃-Ala-Xaa₄-Xaa₅-Xaa₆-NH₂ | R₁ | D-Arg | R₁₁ | R₃ | D-Arg | D-Glu | D-Arg | 26 |
| 27 | R-Xaa₁-Pro-Leu-Gly-Xaa₂-Xaa₃-Ala-Xaa₄-Glu-Xaa₅ | R₁ | R₁₂ | R₈ | R₃ | R₁₂ | R₁₂ | — | 27 |
| 28 | R-Arg-Pro-Leu-Gly-Leu-Xaa₁-Ala-Arg-Glu-Arg-NH₂ | R₂ | R₄ | — | — | — | — | — | 28 |
| 29 | R-Arg-Pro-Gln-Glu-Xaa₁-Xaa₂-Ala-Arg-Glu-Arg-NH₂ | R₁ | R₈ | R₃ | — | — | — | — | 29 |

In Table 3, R₁ through R₁₄ are as follows:

R₁=N=2-[(7-methoxy-2-oxo-2H-chromen-4-yl)acetyl]-.

R₂=N=2-[(5-(2-oxohexahydro-1H-thieno[3,4-d]imidazol-4-yl)pentanoyl]-.

R₃=3-[(2,4-dinitrophenyl)amino]-L-alanine.

R₄=N-6-(4{[3-carboxy-4-(6-hydroxy-3-oxo-3H-xanthen-9-yl)phenyl]amino}-6-chloro-1,3,5-triazin-2-yl)-L-lysine.

R₅=N-6-(4{[3-carboxy-4-(6-hydroxy-3-oxo-3H-xanthen-9-yl)phenyl]amino}-6-[(2-hydroxyethyl)thio]-1,3,5-triazin-2-yl)-L-lysine.

R₆=3-(2-napthyl)-L-alanine.

R₇=O-benzyl-L-tyrosine.

R₈=6-(benzyloxy)-L-norleucine.

R₉=S-(3-phenylpropyl)-L-cysteine.

R₁₀=6-phenoxy-L-norleucine.

R₁₁=S-(4-methoxybenzyl)-L-cysteine.

R₁₂=N-(imidamidyl)-piperidin-3-yl-L-glycine.

R₁₃=N-6-[6-({5-[(4S)-2-oxohexahydro-1H-thieno[3,4-d]imidazol-4-yl)pentanoyl}amino)-hexanoyl]-D-lysine.

R₁₄=S-methyl-L-cysteine.

B. Salts of the Compounds of the Invention

In addition to the compounds described herein, this invention also contemplates salts of such compounds. It is well understood in enzyme chemistry that the particular form of acid/base functional groups on a peptide or protein is determined by the pH of the medium in which the peptide is found, and the respective pKₐ or pK_{b} of the acidic or basic group. At a physiological pH of around 7.0, carboxyl groups of amino acids such as glutamic and aspartic acids naturally exist in the salt or carboxylate form. The functional groups of lysine and arginine, on the other hand, generally contain a protonated nitrogen at such a pH. Unless described otherwise, all references in the specification and claims to a peptide, an amino acid, or an amino acid sequence include both the neutral and the salt forms of the individual peptides, amino acids, and sequences.

C. Use of the Compounds of this Invention

As indicated above, the compounds of this invention tend to be useful for determining the activity of certain proteinases. And because these compounds tend to be selectively cleaved by specific matrix metalloproteases, they are particularly useful in that they allow the determination of the activity of those matrix metalloproteases without interference from other protease (e.g., other MMP and/or constitutive enzyme) activity of proteinases not of interest present in a sample.

In some embodiments, a compound of this invention that is specifically cleaved by a particular matrix metalloprotease of interest (e.g., MMP-2, MMP-9, or MMP-13) is added to a biological sample (e.g., blood, serum, tissue, etc.) that potentially contains that matrix metalloproteinase and one or more other matrix metalloproteinases (e.g., MMP-1 or MMP-7) not of interest. In such an embodiment, only the matrix metalloproteinase of interest will cleave the substrate (to the extent that matrix metalloprotease is present). The substrate, however, will not be cleaved by the other metalloproteinases present in the sample. Thus, any measured MMP activity (detected, for example, by measuring an increase in fluorescence of the sample) will be predominantly (or entirely) from the enzyme for which the compound is specific.

In some particularly preferred embodiments, a compound of this invention is used to determine the activity of MMP-2, MMP-9, or MMP-13 in a biological sample that potentially contains the MMP-2, MMP-9, or MMP-13, as well as one or more other proteases not of interest (e.g., one or both of MMP-1 and MMP-7). Here, a compound of this invention, which is specific for the MMP-2, MMP-9, or MMP-13 (and sparing for MMP-1 and/or MMP-7) is added to the sample. The change of fluorescence in the sample is then measured. Because the compound is specific for the MMP-2, MMP-9, or MMP-13, any increase in fluorescence of the sample will be predominantly (or entirely) reflect the presence of the MMP-2, MMP-9, or MMP-13. Such a measurement may therefore be used to, for example, diagnose or monitor (typically ex-vivo) the status of a disease associated with the MMP-2, MMP-9, or MMP-13. Monitoring the status of such a disease may, in turn, be used to, for example, determine proper dosing or the effectiveness of a treatment.

In some such embodiments, the biological sample is being analyzed to diagnose or monitor a disease associated with MMP-9. Such diseases are believed to include, for example, pathological conditions of the central nervous system associated with nitrosative or oxidative stress. Specific examples of such pathological conditions may be, for example, cerebral ischemia, stroke, or other neurodegenerative diseases. Other diseases believed to be associated with MMP-9 include, for example, eye diseases, such as glaucoma, macular degeneration, and diabetic macular edema. MMP-9 has, for example, been implicated as having a key role in the death of the retinal ganglion cell.

In other embodiments, the biological sample is being analyzed to diagnose or monitor a disease associated with MMP-2 and MMP-9. Such diseases are believed to include, for example, cancer, cardiovascular conditions, and ophthalmologic conditions.

In other embodiments, the biological sample is being analyzed to diagnose or monitor a disease associated with MMP-13. Such diseases are believed to include, for example, cardiovascular conditions and arthritis.

This invention contemplates incorporating a compound of this invention into a kit (typically in a packaged form) to be used to diagnose or monitor the status of a disease.

This invention also contemplates using the compounds of this invention to evaluate the effectiveness of a prospective protease inhibitor. In this instance, the compound is introduced into a sample comprising the protease (typically an MMP, and more typically MMP-2, MMP-9, or MMP-13) and the prospective inhibitor of the protease. A lack of change in fluorescence would indicate inhibition of the protease by the prospective inhibitor. A change of fluorescence, in contrast, would indicate uninhibited protease activity. Example 30 below demonstrates such a use.

This invention further contemplates incorporating a compound of this invention into a kit (typically in a packaged form) to be used to analyze the effectiveness of a prospective protease inhibitor.

D. Preparation of Compounds of this Invention

Compounds useful in accordance with this invention may be prepared by conventional methods of peptide synthesis using the description in this patent, either alone or in combination with techniques generally known in the art. Use of the substrates in the determination of MMP inhibition constants (Example 30) is presented below, as well as synthetic routes for preparing compounds 1-29 (Examples 1-29).

### EXAMPLES

The following examples are merely illustrative, and not limiting to the remainder of this disclosure in any way.

### Example 1

Preparation of N-2-[(7-methoxy-2-oxo-2H-chromen-4-yl)acetyl]-L-arginyl-L-prolyl-L-leucylglycyl-L-leucyl-3-[(2,4-dinitrophenyl)aminol-L-alanyl-L-alanyl-L-arginyl-L-alpha-glutamyl-L-argininamide.

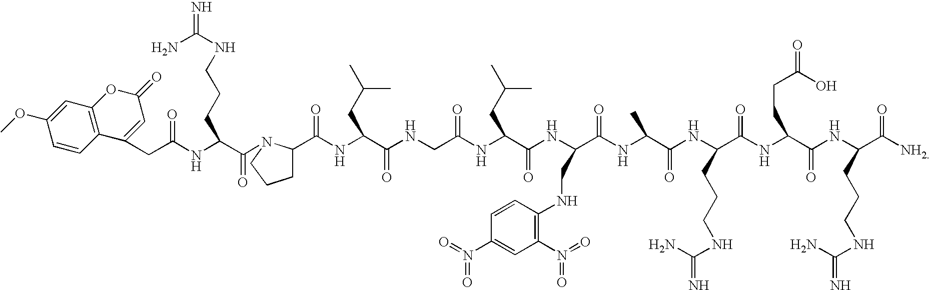

Part A. Preparation of 3-[(2,4-dinitrophenyl)aminol-N-1(9H-fluoren-9-ylmethoxy)carbonyl]alanine.

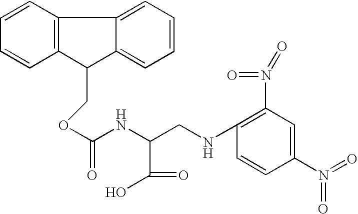

3-[(2,4-dinitrophenyl)amino]-N-[(9H-fluoren-9-ylmethoxy)carbonyl]alanine was prepared following the procedure of Knight, et al., FEBS Letters, 296:263-266 (1992). Fmoc-L-Asn-OH (25.08 g, 71 mmol) (Bachem catalog #B-1045, lot SM511) was dissolved in 175 ml dimethylformamide. Water (35 ml), pyridine (13.2 ml, 143 mmol), and [bis(trifluoroacetoxy)iodo]benzene (46.95 g, 105.9 mmol) (Aldrich 23,213-0, 97% purity) were added, and the mixture was stirred at 20° C. overnight. The mixture was filtered through Whatman filter paper to remove particulates. The volume was reduced under vacuum at 50° C. The concentrated mixture was slowly poured into 1175 ml 1.8 N HCl with stirring. The flask was rinsed with an additional 750 ml water, which was added to the mixture. This solution was stirred 15 min, and extracted with 2400 ml diethyl ether in 4 portions. The aqueous solution was slowly adjusted to pH 7 by the gradual addition of Na₂CO₃ (111.5 g, 1052 mmol). NaHCO₃ (11.86g, 141 mmol), ethanol (1788 ml), and 2,4-dinitrofluoro-benzene (13.139 g, 70.6 mmol) (Aldrich D19,680-0) were added, and the mixture was stirred at 20° C. overnight. Concentrated HCl (190 ml) was added with stirring to adjust the pH to pH 1. Dark red-brown precipitate was collected on a sintered glass filter, and washed with approximately 100 ml each, 0.1 N HCl, ethanol, and diethyl ether. The residue was dried under vacuum. Yield was 76% (26.5 g, 53.9 mmol), 98% purity by analytical reversed phase HPLC. Electrospray mass spectrometry gave M+H 493.4 and M+Na 515.4.

Part B. Preparation of L-alanyl-L-arginyl-L-glutamyl-L-arginyl-resin.

L-alanyl-L-arginyl-L-glutamyl-L-arginyl-resin was prepared attached to Applied Biosystems Fmoc-Amide-Resin (Product number 401435) using the Applied Biosystems model 433A Peptide Synthesizer and the manufacturer's reagents and reaction vessel designed for the 0.25 mM synthesis scale. The manufacturer's preprogrammed cycles were modified to increase each cycle reaction time by 30 min.

Part C. Preparation of 3-[(2,4-dinitrophenyl)amino]-N-[(9H-fluoren-9-ylmethoxy)carbonyl]alanyl-L-alanyl-L-arginyl-L-glutamyl-L-arginyl-resin.

The product from Part A (1 mmol, 0.492 g) was dissolved in 2 ml dimethylformamide. To this solution was added 1 mmol 1-Hydroxy-7-azabenzo-triazole (HOAT, Aldrich, Product 44,545-2), 1 mmol O-(7-Azabenzotriazol-1yl)-N,N,N′,N′-tetramethyluronium hexafluorophosphate (HATU, Aldrich, Product 44,545-0) and 0.25 mmol of the product from Part B that had been swollen with 2.5 ml CH₂Cl₂ and diluted to 5 ml with dimethylformamide solvent. N,N-Diisopropylethylamine (4 mmol, 0.7 ml) (DIEA, Applied Biosystems, Product 400136) was added, the suspension was diluted to 10 ml final volume, and agitated overnight. The resin was washed on a coarse glass scintered filter with dimethylformamide (20 ml×2) alternated with CH₂Cl₂ (20 ml×2) for 2 cycles. Unreacted amino groups remaining on the resin were then capped by dripping a solution through the resin on a coarse glass scintered filter containing 10 ml acetic anhydride (106 mmol), 10 ml N,N-Diisopropylethylamine (57 mmol) and 30 ml dimethylformamide. The resin was washed again as described, and returned to the automated synthesis reaction vessel for Fmoc removal and chain elongation.

Part D. Preparation of L-arginyl-L-prolyl-L-leucyl-glycyl-L-leucyl-3-[(2,4-dinitrophenyl)amino]-alanyl-L-alanyl-L-arginyl-L-glutamyl-L-arginyl-resin.

The product from Part C above was elongated still attached to the resin using the Applied Biosystems Model 433A Synthesizer and the manufacturer's reagents and reaction vessel designed for the 0.25 mM synthesis scale. The manufacturer's preprogrammed cycles were modified to increase each cycle reaction time by 30 min. The amino acids L-leucine, glycine, L-leucine, L-proline, and L-arginine were added in order. The amino terminal Fmoc was removed prior to removal of the resin from the machine.

Part E. Preparation of N-2-[(7-methoxy-2-oxo-2H-chromen-4-yl)acetyl]-L-arginyl-L-prolyl-L-leucylglycyl-L-leucyl-3-[(2,4-dinitrophenyl)amino]-L-alanyl-L-alanyl-L-arginyl-L-alpha-glutamyl-L-argininamide (C₆₆H₉₉N₂₃O₂₀).

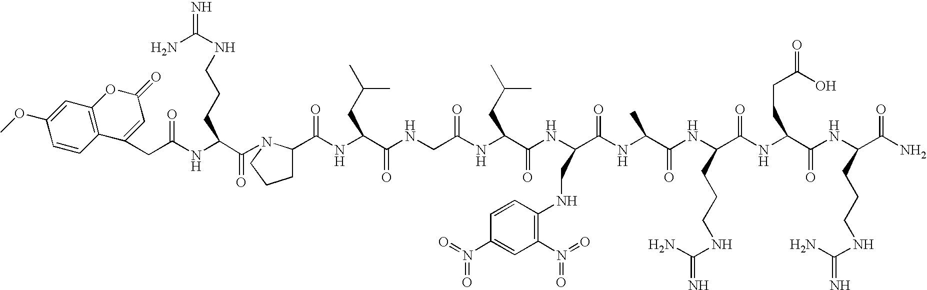

The 7-methoxycoumarin-4-acetic acid reagent (1 mmol, 0.234 g) (Aldrich, Product 23,519-9) was suspended in 5 ml dimethylformamide. To this suspension was added 1 mmol 1-Hydroxy-7-azabenzo-triazole (HOAT, Aldrich, Product 44,545-2), 1 mmol O-(7-Azabenzotriazol-1yl)-N,N,N′,N′-tetramethyluronium hexafluorophosphate (HATU, Aldrich, Product 44,545-0). This suspension was then added to 0.25 mmol of the product from Part D that had been swollen with 2.5 ml CH₂Cl₂, and the suspension was diluted to 10 ml with dimethyl-formamide solvent. N,N-Diisopropylethylamine (2 mmol, 0.35 ml) (DIEA, Applied Biosystems, Product 400136) was added in 2 equal aliquots over 30 min, and the suspension was agitated overnight. The resin was washed on a coarse glass scintered filter with dimethylformamide (20 ml×2) alternated with CH₂Cl₂ (20 ml×2) for 2 cycles. The resin was dried under vacuum, and cleaved with 5 ml of a solution of trifluoroacetic acid: H₂0: triisopropylsilane 1:18:1 for 2-3 hr. The peptide-containing solution was filtered through a glass frit into a total volume of 100 ml of diethylether, and pelleted at 5000 rpm for 5 min. The pellet was washed with ether once and air dried. The pellet was then dissolved in dimethylsulfoxide, and the peptide was purified on a C18 reverse phase column developed with a 5-95% acetonitrile gradient in water. Fractions containing >95% pure target peptide substrate were lyophilized to dryness. Overall yield was 74% (0.285 g, 0.186 mmol), 99% purity by analytical reversed phase HPLC. Electrospray mass spectrometry gave M+2H 768.4, M+3H 512.8 and M+H+Na 779.5, corresponding to the expected exact mass of 1533.74.

### Example 2

Preparation of N-2-[5-(2-oxohexahydro-1H-thieno[3,4-d]imidazol-4-yl)pentanoyl]-L-arginyl-L-prolyl-L-leucylglycyl-L-leucyl-N-6-(4-{[3-carboxy-4-(6-hydroxy-3-oxo-3H-xanthen-9-yl)phenyl]amino}-6-chloro-1,3,5-triazin-2-yl)-L-lysyl-L-alanyl-L-arginyl-L-alpha-glutamyl-L-argininamide.

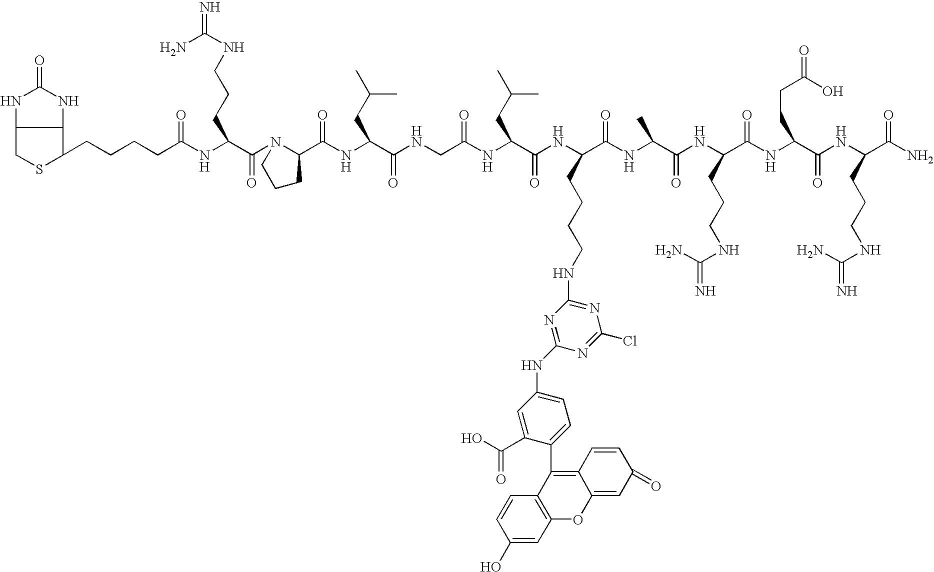

Part A. Preparation of L-arginyl-L-prolyl-L-leucylglycyl-L-leucyl-L-lysyl-L-alanyl-L-arginyl-L-alpha-glutamyl-arginyl-resin.

L-arginyl-L-prolyl-L-leucylglycyl-L-leucyl-L-lysyl-L-alanyl-L-arginyl-L-alpha-glutamyl-arginyl-resin was prepared attached to Applied Biosystems Fmoc-Amide-Resin (Product number 401435) using the Applied Biosystems model 433A Peptide Synthesizer. The manufacturer's preprogrammed cycles were modified to increase each cycle reaction time by 30 min.

Part B. Preparation of N-2-{5-[(3aS,4S,6aR)-2-oxohexahydro-1H-thieno[3,4-d]imidazol-4-yl]pentanoyl}-L-arginyl-L-prolyl-L-leucylglycyl-L-leucyl-L-lysyl-L-alanyl-L-arginyl-L-alpha-glutamyl-L-arginyl-resin.

5-[(3aS,4S,6aR)-2-oxohexahydro-1H-thieno[3,4-d]imidazol-4-yl]pentanoic acid (1 mmol, 0.244 g) was dissolved in 10 ml dimethylformamide. To this solution was added 1 mmol (0.135 g) 1-hydroxybenzotriazole hydrate (HOBT, Aldrich, Product 15726-0), 1 mmol (0.442 g) (benzotriazol-lyloxy)tr(dimethylamino)phosphonium hexafluorophosphate (BOP, Castros Reagent, Perseptive Biosystems, Product GEN076503), and 0.25 mmol of the product from Part A that had been swollen with CH₂Cl₂. N,N-Diopropylethylamine (4 mmol, 0.7 ml) (DIEA, Applied Biosystems, Product 400136) was added, and the suspension was agitated overnight. The resin was washed on a coarse glass scintered filter with dimethylformamide (20 ml×2) alternated with CH₂Cl₂ (20 ml×2) for 2 cycles. The above protocol was repeated to ensure quantitative coupling.

Part C. Preparation of N-2-{5-[(3aS,4S,6aR)-2-oxohexahydro-1H-thieno[3,4-d]imidazol-4-yl]pentanoyl}-L-arginyl-L-prolyl-L-leucylglycyl-L-leucyl-L-lysyl-L-alanyl-L-arginyl-L-alpha-glutamyl-L-arginamide (C₆₁H₁₀₉N₂₃O₁₄S).

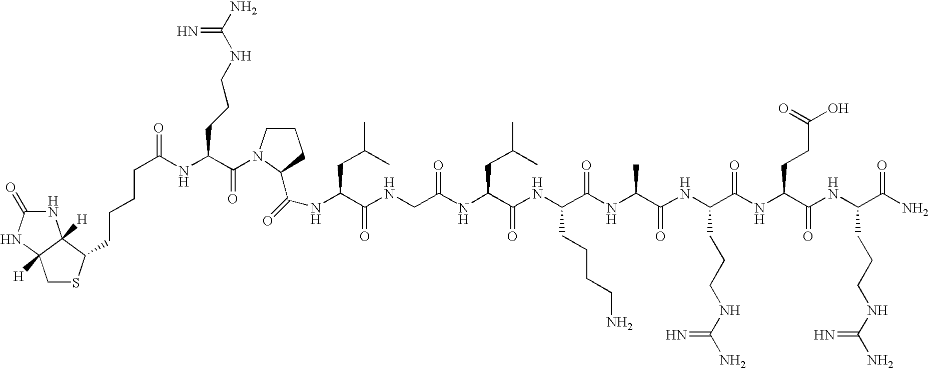

The biotinylated peptide from Part B was dried under reduced pressure, and cleaved with 5 ml of a solution of trifluoroacetic acid (4.6 ml), ethanedithiol (0.125 ml), thioanole (0.25 ml), and H₂O (0.25 ml) for 6 hr at room temperature. The peptide-containing solution was filtered through a glass frit into a total volume of 100 ml of diethylether, and pelleted at 5000 rpm for 5 min. The pellet was washed with ether twice, and dried under vacuum. The pellet was then dissolved in dimethylsulfoxide, and the peptide was purified on a C18 reverse phase HPLC column developed with a 5-95% acetonitrile gradient in water. Fractions containing >95% pure target peptide substrate were pooled, and lyophilized to dryness. Overall yield was 70% (0.25 g, 0.176 mmol), 99% purity by analytical reversed phase HPLC. Electrospray mass spectrometry gave M+2H 710.9 and M+3H 474.3, corresponding to the expected exact mass of 1419.82.

Part D. Preparation of N-2-[5-(2-oxohexahydro-1H-thieno[3,4-dimidazol-4-yl)pentanoyl]-L-arginyl-L-prolyl-L-leucylglycyl-L-leucyl-N-6-(4-[3-carboxy-4-(6-hydroxy-3-oxo-3H-xanthen-9-yl)phenyl]amino}-6-chloro-1,3,5-triazin-2-yl)-L-lysyl-L-alanyl-L-arginyl-L-alpha-glutamyl-L-argininamide (C₈₄H₁₂₀ClN₂₇O₁₉S).

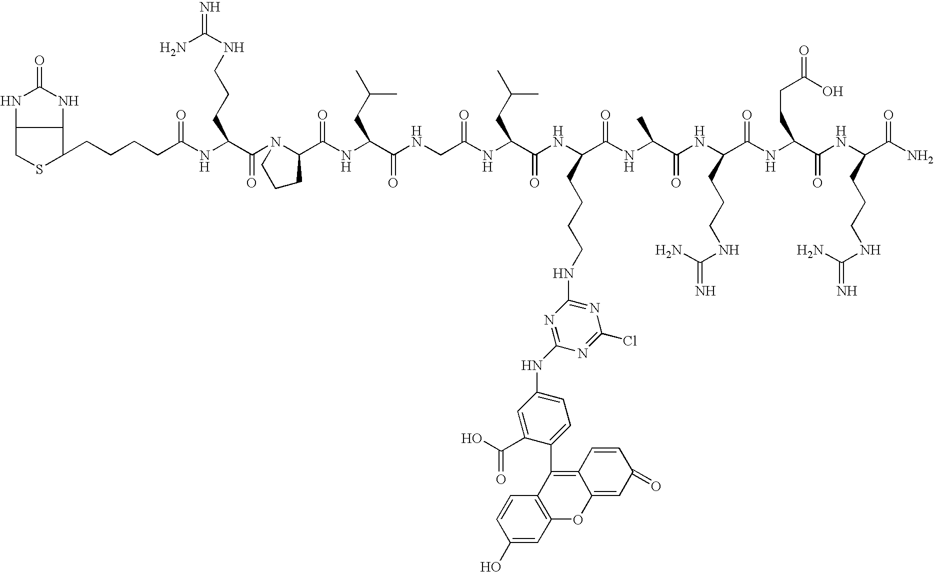

The 5-(4,6-dichlorotriazinyl)aminofluorescein, hydrochloride reagent (0.94 mmol, 0.5 g) (Molecular Probes, Eugene, Oreg., Product D-16) was added to a 10 ml solution of the product from Part C (1 g, 0.70 mmol) dissolved in dimethylformamide with 10 mmol (1.24 g) N,N-Diopropylethylamine (DIEA, Applied Biosystems, Product 400136). The reaction was allowed to proceed at room temperature overnight with gentle agitation. The reaction mixture was then dripped into 400 ml diethylether, and pelleted at 5000 rpm for 5 min. The pellet was washed with ether twice, and dried under vacuum. The pellet was then dissolved in 50% acetic acid, and lyophilized to dryness (1.628 g). The powder was dissolved in dimethylsulfoxide, and purified on a C18 reverse phase HPLC column developed with a 5-95% acetonitrile gradient in water. Fractions containing >95% pure target peptide substrate were pooled, and lyophilized to dryness. Yield was 40% (0.53 g, 0.28 mmol), 99% purity by analytical reversed phase HPLC. Electrospray mass spectrometry gave M+2H 940.4379, corresponding to the expected exact mass of 1877.8663.

### Example 3

Preparation of N-2-[5-(2-oxohexahydro-1H-thieno[3,4-d]imidazol-4-yl)pentanoyl]-L-arginyl-L-prolyl-L-leucylglycyl-L-leucyl-N-6-{4-{[3-carboxy-4-(6-hydroxy-3-oxo-3H-xanthen-9-yl)phenyl]amino}-6-[(2-hydroxyethyl)thio]-1,3,5-triazin-2-yl)-L-lysyl-L-alanyl-L-arginyl-L-alpha-glutamyl-L-argininamide (C₈₆H₁₂₅N₂₇O₂₀S₂).

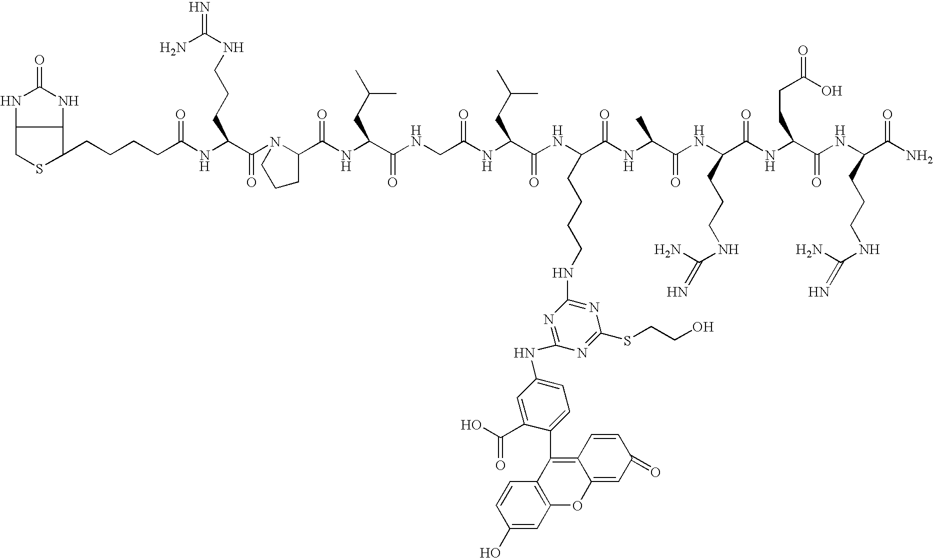

To a suspension of the product from Example 2, Part D (0.031 g, 0.016 mmol) in 4.5 ml tetrahydrofuran was added 0.871 mmol (0.108 g) N,N-Diopropylethylamine (DIEA, Applied Biosystems, Product 400136) and 2.96 mmol (0.231 g) 2-mercaptoethanol. The reaction was stirred overnight at room temperature. 1 ml (22% of total) was precipitated in 10 ml ether, and pelleted at 5000 rpm for 5 min. The pellet was dissolved in dimethylsulfoxide, and the peptide was purified on a C18 reverse phase column developed with a 5-95% acetonitrile gradient in water. Fractions containing >95% pure target peptide substrate were lyophilized to dryness. Overall yield was 75% (0.006 g, 0.003 mmol). Electrospray mass spectrometry gave M+2H 961.5 and M+3H 641.3, corresponding to the expected exact mass of 1919.9036. Fluorescence excitation scans were performed at fixed emission 515 nM, and emission scans were performed at fixed excitation 495 nM. Optimum was found to be unchanged from that of the product from Example 2, Part D.

### Example 4

Preparation of N-[5-(2-oxohexahydro-1H-thieno[3,4-d]imidazol-4-yl)pentanoyl]-L-alpha-glutamyl-L-prolyl-L-leueylglycyl-L-leucyl-N-6-(4-{[3-carboxy-4-(6-hydroxy-3-oxo-3H-xanthen-9-yl)phenyl]amino}-6-chloro-1,3,5-triazin-2-yl)-L-lysyl-L-alanyl-L-alpha-glutamyl-L-arginyl-L-alpha-glutamine.

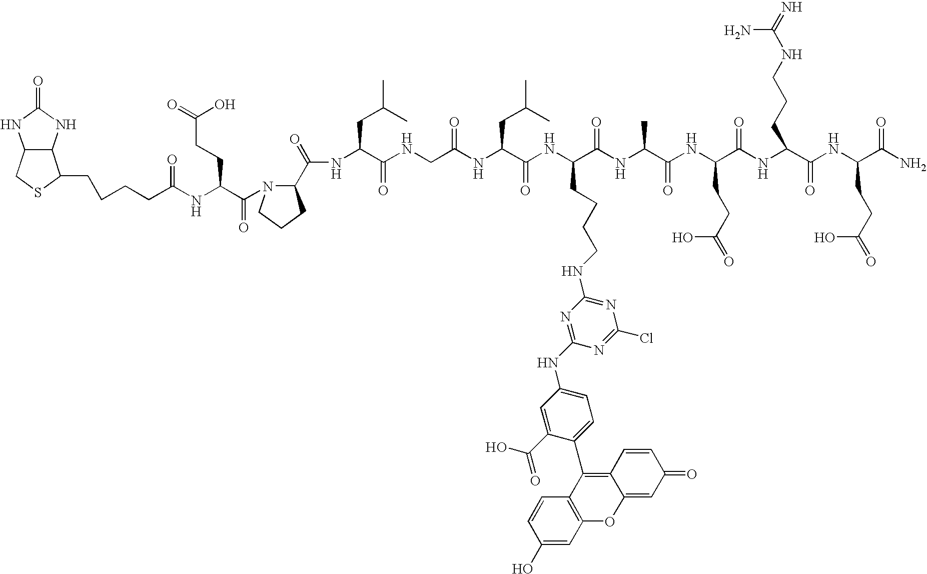

Part A. Preparation of L-alpha-glutamyl-L-prolyl-L-leueylglycyl-L-leueyl-L-lysyl-L-alanyl-L-alpha-glutamyl-L-arginyl-L-alpha-glutamyl-resin.

L-alpha-glutamyl-L-prolyl-L-leucylglycyl-L-leucyl-L-lysyl-L-alanyl-L-alpha-glutamyl-L-arginyl-L-alpha-glutamyl-resin was synthesized attached to Applied Biosystems Fmoc-Amide-Resin (Product number 401435) using the Applied Biosystems model 433A Peptide Synthesizer. The manufacturer's preprogrammed cycles were modified to increase each cycle reaction time by 30 min.

Part B. Preparation of N-[5-(2-oxohexahydro-1H-thieno[3,4-d]imidazol-4-yl)pentanoyl]-L-alpha-glutamyl-L-prolyl-L-leuylglycyl-L-leuyl-L-lysyl-L-alanyl-L-alpha-glutamyl-L-arginyl-L-alpha-glutamyl-resin.

5-[(3aS,4S,6aR)-2-oxohexahydro-1H-thieno[3,4-d]imidazol-4-yl]pentanoic acid(d-Biotin, Sigma, Product B-4501) was manually conjugated to the resin bound product from Part A using the procedure described in Example 2, Part B.

Part C. Preparation of N-[5-(2-oxohexahydro-1H-thieno[3,4-d]imidazol-4-yl)pentanoyl]-L-alpha-glutamyl-L-prolyl-L-leucylglycyl-L-leucyl-L-lysyl-L-alanyl-L-alpha-glutamyl-L-arginyl-L-alpha-glutamine (C₅₉H₉₉N₁₇O₁₈S).

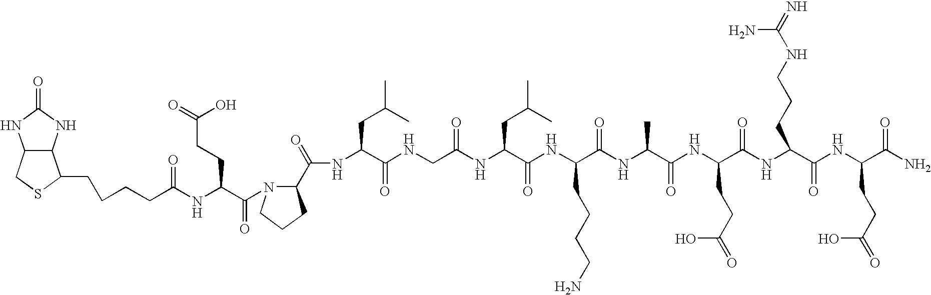

The biotinylated product from Part B was dried under reduced pressure, and cleaved with 5 ml of a solution of trifluoroacetic acid (4.6 ml), ethanedithiol (0.125 ml), thioanole (0.25 ml) and H₂O (0.25 ml) for 6 hr at room temperature. The peptide-containing solution was filtered through a glass frit into a total volume of 100 ml of diethylether, and pelleted at 5000 rpm for 5 min. The pellet was washed with ether twice, and dried under vacuum. The pellet was then dissolved in dimethylsulfoxide, and the peptide was purified on a C18 reverse phase HPLC column developed with a 5-95% acetonitrile gradient in water. Fractions containing >95% pure target peptide substrate were pooled, and lyophilized to dryness. Overall yield was 65% (0.22 g, 0.163 mmol), 95% purity by analytical reversed phase HPLC. Electrospray mass spectrometry gave M+2H 683.8, corresponding to the expected exact mass of 1365.

Part D. Preparation of N-[5-(2-oxohexahydro-1H-thieno[3,4-d]imidazol-4-yl)pentanoyl]-L-alpha-glutamyl-L-prolyl-L-leucylglycyl-L-leucyl-N-6-(4-{[3-carboxy-4-(6-hydroxy-3-oxo-3H-xanthen-9-yl)phenyl]amino}-6-chloro-1,3,5-triazin-2-yl)-L-lysyl-L-alanyl-L-alpha-glutamyl-L-arginyl-L-alpha-glutamine (C₈₂H₁₁₀N₂₁O₂₃S).

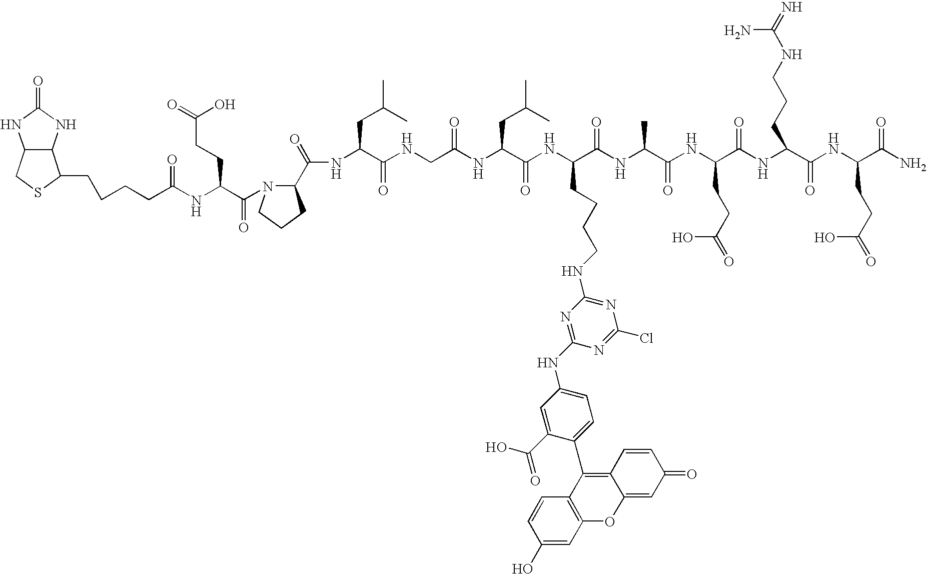

5-(4,6-dichlorotriazinyl)aminofluorescein, hydrochloride reagent (0.2 mmol, 0.106 g) (Molecular Probes, Eugene, Oreg., Product D-16) was added to a 5 ml solution of the product from Part C (0.223 g, 0.163 mMoles) dissolved in dimethylformamide with 2 mmol (0.25 g) N,N-Diopropylethylamine (DIEA, Applied Biosystems, Product 400136). The reaction was allowed to proceed at room temperature overnight with gentle agitation. The reaction mixture was then dripped into 100 ml diethylether, and pelleted at 5000 rpm for 5 min. The pellet was washed with ether twice, and dried under vacuum. The pellet was dissolved in dimethylsulfoxide, and purified on a C18 reverse phase HPLC column developed with a 5-95% acetonitrile gradient in water. Fractions containing >95% pure target peptide substrate were pooled, and lyophilized to dryness. Yield was 72% (0.215 g, 0.12 mmol), 99% purity by analytical reversed phase HPLC. Electrospray mass spectrometry gave M+2H 913.9, corresponding to the expected exact mass of 1823.00.

### Example 5

Preparation of N-[5-(2-oxohexahydro-1H-thieno[3,4-d]imidazol-4-yl)pentanoyl]-L-alpha-glutamyl-L-prolyl-L-leucylglycyl-L-leucyl-N˜6˜-{4-{[3-carboxy-4-(6-hydroxy-3-oxo-3H-xanthen-9-yl)phenyl]amino}-6-[(2-hydroxyethyl)thiol]-1,3,5-triazin-2-yl}-L-lysyl-L-alanyl-L-alpha-glutamyl-L-arginyl-L-alpha-glutamine (C₈₄H₁₁₅N₂₁O₂₄S₂).

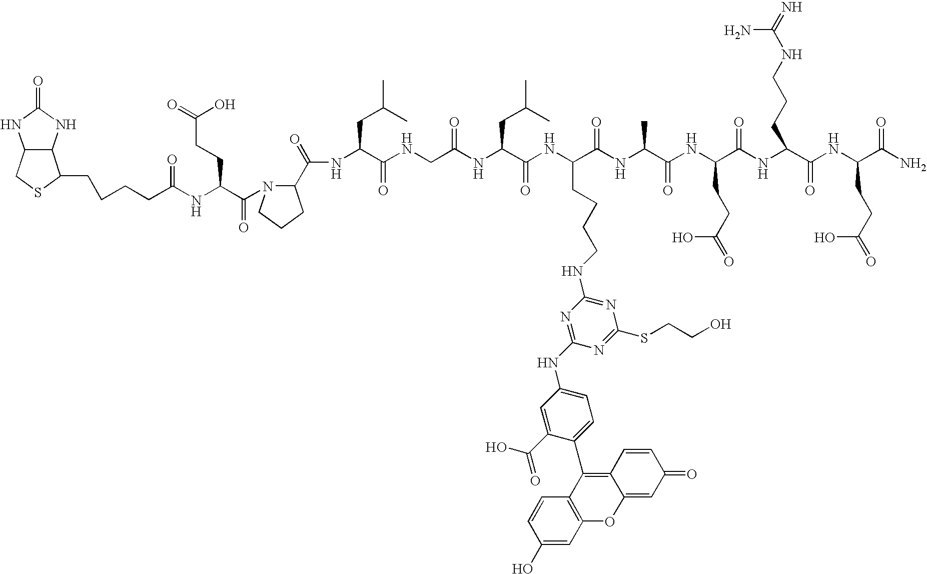

To a suspension of the product from Example 4 (0.070 g, 0.039 mmol) in 5 ml dimethylformamide was added 0.11 mmol (0.015 g) K₂CO₃ and 5.1 mmol (0.4 g) 2-mercaptoethanol. The reaction was stirred ˜40 hr at room temperature. The sample was dripped into 45 ml ether, and pelleted at 5000 rpm for 5 min. The pellet was dissolved in dimethylsulfoxide, and the peptide is purified on a C18 reverse phase column developed with a 5-95% acetonitrile gradient in water. Fractions containing >95% pure target peptide substrate were lyophilized to dryness. Overall yield was 74% (0.054 g, 0.029 mmol). Electrospray mass speetrometry gave M+2H 933.9, corresponding to the expected exact mass of 1865.00. Fluorescence excitation scans were performed at fixed emission 515 nM. Emission scans were performed at fixed excitation 495 nM. Optimum was found to be unchanged from that of Example 2. Molecular weight 1867.11. Exact mass 1865. Molecular formula C₈₄H₁₁₅N₂₁O₂₄S₂. Molecular composition C 54.04% H 6.21% N 15.75% O 20.57% S 3.43%.

### Example 6

Preparation of 1-[(7-methoxy-2-oxo-2H-chromen-4-yl)acetyl]-L-prolyl-L-leucylglycyl-3-(2-naphthyl)-L-alanyl-3-[(2,4-dinitrophenyl)amino]-L-alanyl-L-alanyl-L-argininamide.

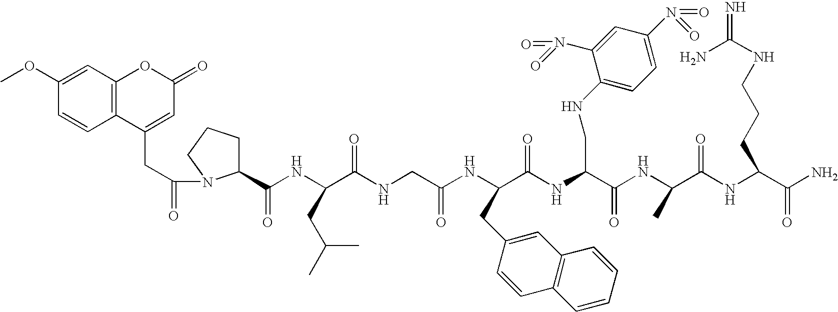

Part A. Preparation of L-alanyl-L-arginyl-resin.

L-alanyl-L-arginyl-resin was synthesized attached to Applied Biosystems Fmoc-Amide-Resin (Product number 401435) using the Applied Biosystems model 433A Peptide Synthesizer and the manufacturer's reagents and reaction vessel designed for the 0.25 mM synthesis scale. The manufacturer's preprogrammed cycles were modified to increase each cycle reaction time by 30 min.

Part B. Preparation of 3-[(2,4-dinitrophenyl)amino]-L-alanyl-L-alanyl-L-arginyl-resin.

The product from Example 1, Part A (1 mmole, 0.492 g) was dissolved in 2 ml dimethylformamide. To this solution was added 1 mmol 1-Hydroxy-7-azabenzo-triazole (HOAT, Aldrich, Product 44,545-2), 1 mmol O-(7-Azabenzotriazol-1-yl)-N,N,N′,N′-tetramethyluronium hexafluorophosphate (HATU, Aldrich, Product 44,545-0), and 0.25 mmol of the product from Part A that had been swollen with 2.5 ml CH₂Cl₂, and diluted to 5 ml with dimethylformamide solvent. N,N,-Diopropylethylamine (4 mmol, 0.7 ml) (DIEA, Applied Biosystems, Product 400136) was added, and the suspension was diluted to 10 ml final volume, and agitated overnight. The resin was washed on a coarse glass scintered filter with dimethylformamide (20 ml×2) alternated with CH₂Cl₂ (20 ml×2) for 2 cycles. Unreacted amino groups remaining on the resin were then capped by dripping a solution through the resin on a coarse glass scintered filter containing 10 ml acetic anhydride (106 mmol), 10 ml N,N,-Diopropylethylamine (57 mmol) and 30 ml dimethylformamide. The resin was washed again as described, and returned to the automated synthesis reaction vessel for Fmoc removal and chain elongation.

Part C. Synthesis of L-prolyl-L-leucylglycyl-3-(2-naphthyl)-L-alanyl-3-[(2,4-dinitrophenyl)amino]-L-alanyl-L-alanyl-L-arginyl-resin.

The product from Part B was elongated still attached to the resin using the Applied Biosystems Model 433A Synthesizer and the manufacturer's reagents and reaction vessel designed for the 0.25 mmol scale. The manufacturer's preprogrammed cycles were modified to increase each cycle reaction time by 30 min. The amino acids L-3-(2-naphthyl)-alanine, glycine, L-leucine and L-proline were added in order, and the amino terminal Fmoc was removed prior to removal of the resin from the machine.

Part D. Preparation of 1-[(7-methoxy-2-oxo-2H-chromen-4-yl)acetyl]-L-prolyl-L-leucylglycyl-3-(2-naphthyl)-L-alanyl-3-[(2,4-dinitrophenyl)amino]-L-alanyl-L-alanyl-L-argininamide (C₅₆H₆₈N₁₄O₁₅).

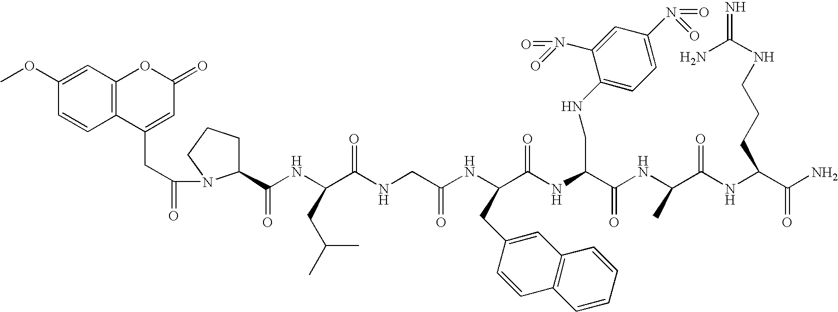

The 7-methoxycoumarin-4-acetic acid reagent (0.105 g, 0.45 mmol) (Aldrich, Product 23,519-9) was suspended in 5 ml dimethylformamide. To the suspension was added 0.45 mmol (0.061 g) 1-hydroxybenzotriazole hydrate (HOBT, Aldrich, Product 15726-0), 0.45 mmol (0.234 g) benzotriazole-1-yloxy-tr-pyrrolidino-phosphonium hexafluorophosphate (PyBOP, Novabiochem, Product 01-62-0016), and 0.25 mmol of the product from Part C that had been swollen with CH₂Cl₂. N,N-Diopropylethylamine (1 mmol, 0.174 ml) (DIEA, Applied Biosystems, Product 400136) was added, and the suspension was agitated overnight. The resin was washed on a coarse glass scintered filter with dimethylformamide (20 ml×2) alternated with CH₂Cl₂ (20 ml×2) for 2 cycles. The above protocol was repeated two times to ensure quantitative coupling. The resin was dried under vacuum, and cleaved with 5 ml of a solution of triflouroacetic acid:H₂O:triopropylsilane 18:1:1 for 2-3 hr. The peptide-containing solution was filtered through a glass frit into a total volume of 100 ml of diethylether, and pelleted at 5000 rpm for 5 min. The pellet was washed with ether once, and air dried. The pellet was then dissolved in dimethylsulfoxide, and the peptide was purified on a C18 reverse phase HPLC column developed with a 5-95% acetonitrile gradient in water. Fractions containing >95% pure target peptide substrate were pooled, and lyophilized to dryness. Overall yield was 5% (0.013 g, 0.011 mMoles), 98% purity by analytical reversed phase HPLC. Poor yield was found to be due to incorporation of an incompletely protected arginine reagent leading to incorporation of multiple residues. Electrospray mass spectrometry gave M+H 1177.5, corresponding to the expected exact mass of 1176.4987.

### Example 7

Preparation of 1-[(7-methoxy-2-oxo-2H-chromen-4-yl)acetyl]-L-prolyl-L-leucylglycyl-O-benzyl-L-tyrosyl-3-[(2,4-dinitrophenyl)amino]-L-alanyl-L-alanyl-L-argininamide.

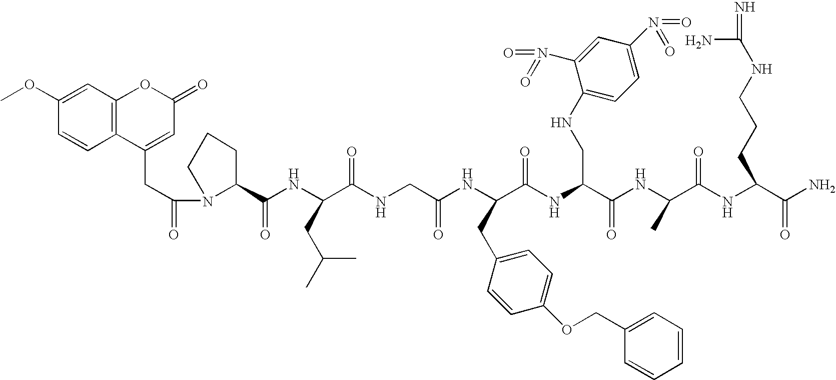

Part A. Preparation of L-prolyl-L-leucylglycyl-O-benzyl-L-tyrosyl-3-[(2,4-dinitrophenyl)aminol-L-alanyl-L-alanyl-L-arginyl-resin.

The product from Example 6, Part B was elongated still attached to the resin using the Applied Biosystems Model 433A Synthesizer and the manufacturer's reagents and reaction vessel designed for the 0.25 mmol scale. The manufacturer's preprogrammed cycles were modified to increase each cycle reaction time by 30 min. The amino acids O-benzyl-L-tyrosine, glycine, L-leucine, and L-proline were added in order, and the amino terminal Fmoc was removed prior to removal of the resin from the machine.

Part B. Preparation of 1-[(7-methoxy-2-oxo-2H-chromen-4-yl)acetyl]-L-prolyl-L-leucylglycyl-O-benzyl-L-tyrosyl-3-[(2,4-dinitrophenyl)amino]-L-alanyl-L-alanyl-L-argininamide (C₅₉H₇₂N₁₄O₁₆).

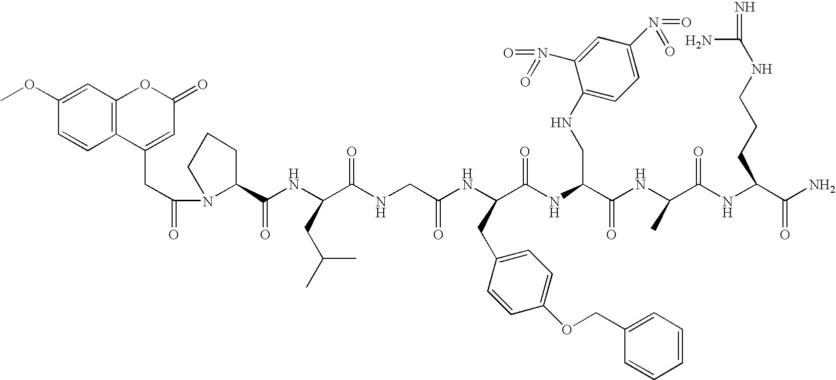

The 7-methoxycoumarin-4-acetic acid reagent (0.105 g, 0.45 mmol) (Aldrich, Product 23,519-9) was suspended in 5 ml dimethylformamide. To the suspension was added 0.45 mmol (0.061 g) 1-hydroxybenzotriazole hydrate (HOBT, Aldrich, Product 15726-0), 0.45 mmol (0.234 g) benzotriazole-1-yloxy-tr-pyrrolidino-phosphonium hexafluorophosphate (PyBOP, Novabiochem, Product 01-62-0016) and 0.25 mmol of the product from Part A that had been swollen with CH₂Cl₂. N,N-Diopropylethylamine (1 mmol, 0.174 ml) (DIEA, Applied Biosystems, Product 400136) was added, and the suspension was agitated overnight. The resin was washed on a coarse glass scintered filter with dimethylformamide (20 ml×2) alternated with CH₂Cl₂ (20 ml×2) for 2 cycles. The above protocol was repeated two times to ensure quantitative coupling. The resin was dried under vacuum, and cleaved with 5 ml of a solution of triflouroacetic acid:H₂O:triopropylsilane 18:1:1 for 2-3 hr. The peptide-containing solution was filtered through a glass frit into a total volume of 100 ml of diethylether, and pelleted at 5000 rpm for 5 min. The pellet was washed with ether once, and air dried. The pellet was then dissolved in dimethylsulfoxide, and the peptide was purified on a C18 reverse phase HPLC column developed with a 5-95% acetonitrile gradient in water. Fractions containing >95% pure target peptide substrate were pooled, and lyophilized to dryness. Overall yield was 5% (0.013 g, 0.011 mMoles), 98% purity by analytical reversed phase HPLC. Poor yield was found to be due to incorporation of an incompletely protected arginine reagent leading to incorporation of multiple residues. Electrospray mass spectrometry gave M+H 1235.5, corresponding to the expected exact mass of 1232.5249.

### Example 8

Preparation of 1-[(7-methoxy-2-oxo-2H-chromen-4-yl)acetyl]-L-prolyl-L-leucylglycyl-6-(benzyloxy)-L-norleucyl-3-[(2,4-dinitrophenyl)amino]-L-alanyl-L-alanyl-L-argininamide.

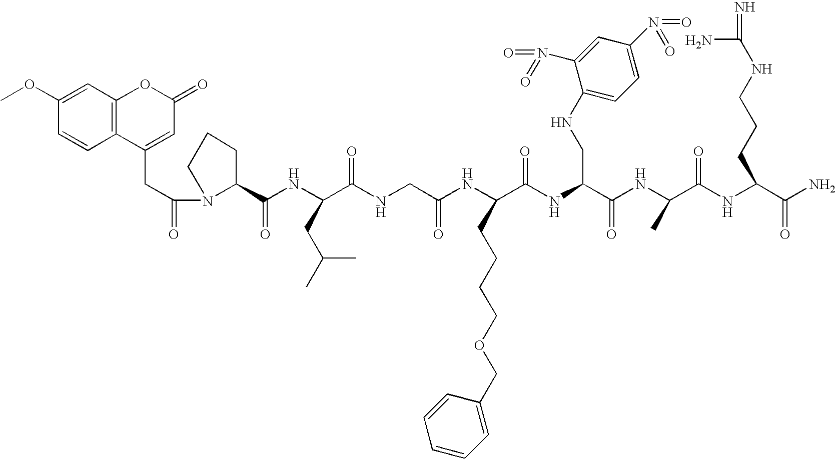

Part A. Preparation of N-[(9H-fluoren-9-ylmethoxy)carbonyl]-6-hydroxy-L-norleucine.

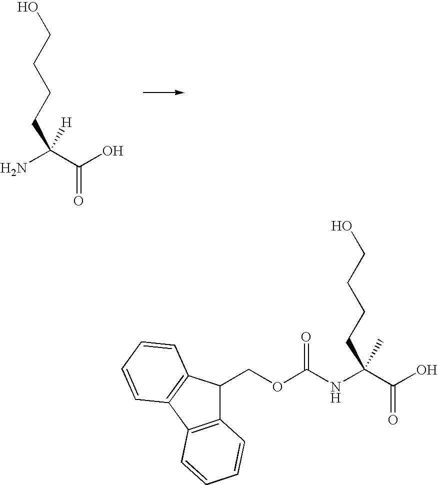

L-6-Hydroxynorleucine (Chemsampco, Gray Court, S.C.) (5 g, 34 mmol) was dissolved in 90 ml of a 10% solution of Na₂CO₃ in H₂O. Dioxane (51 ml) was added, and the mixture chilled to 4° C. in an ice bath. 9-Fluorenylmethyl chloroformate (Sigma Chemical, St. Lou, Mo.) (8.925 g, 34.5 mmol) was added in small portions, and stirring continued at 4° C. for 3 hr. The reaction was allowed to equilibrate to room temperature, and stirring was continued overnight. The reaction mixture was poured into 1700 ml H₂O, and extracted with six 300 ml aliquots of ether. The aqueous solution was then cooled in an ice water bath, and acidified with concentrated HCl to pH 2. The mixture was kept refrigerated overnight to facilitate precipitation of product. The precipitate was collected on a scintered glass filter, washed with H₂O, and dried under vacuum to afford 10.89 g (29.5 mmol) of an off-white solid for an 87% yield. By electropspray mass spectroscopy, the product gave M+H 370 and M+NH₄ 387, constent with target. Purity was 94% by analytical HPLC.

Part B. Preparation of 6-(benzyloxy)-N-(9H-fluoren-9-ylmethoxy)carbonyl]-L-norleucine and 1-benzyl N-[(9H-fluoren-9-ylmethoxy)carbonyl]-6-hydroxynorleucinate.

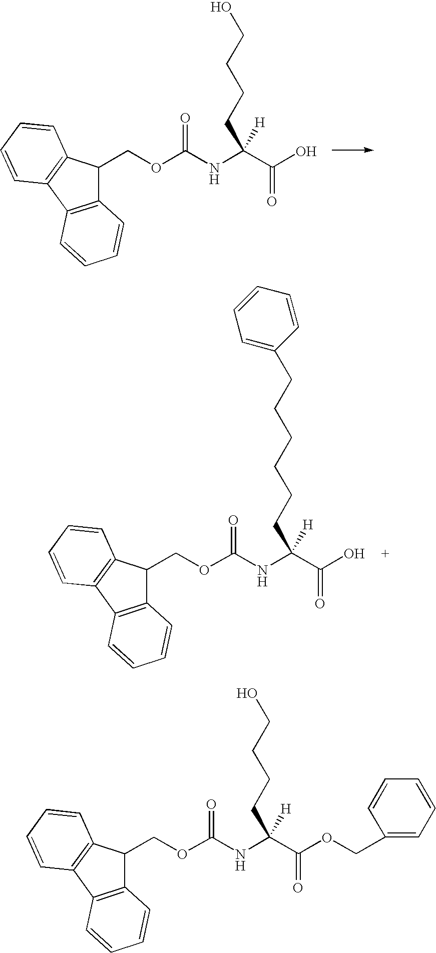

To a suspension of product of Part A (2 g, 5.4 mmol) in anhydrous diethylether (40 ml) was added 1.3 ml (1.76 g, 7 mmol, 1.28 equivalents)benzyl 2,2,2-trichloroacetimidate (Aldrich, Milwaukee, Wis.) followed by 0.4 ml boron trifluoride diethyl etherate BF₃Et₂O (Aldrich, Milwaukee, Wis.). The reaction was stirred at 0° C. for 2 hr. The ether layer was diluted with H₂O until the precipitate dissolved, and was washed twice with saturated NaHCO₃. The aqueous layer was acidified to pH 2 with concentrated HCl, extracted with ethylacetate (500 ml), dried over Na₂SO₄, and evaporated to dryness under reduced pressure. The products were resolved on reverse phase HPLC, frozen on dry ice, and lyophilized to dryness. Recovered 0.475 g highly purified starting material (24%) and two well-resolved major species, both C₂₈H₂₉NO₅ which both gave M+H 460.3 and M+NH₄ 477.3 by electrospray mass spectrometry. The benzyl N-[(9H-fluoren-9-ylmethoxy) carbonyl]-6-hydroxy-L-norleucinate (0.227 g, 0.495 mmol, 9.2% yield) eluted before the 6-(benzyloxy)-N-[(9H-fluoren-9-ylmethoxy)carbonyl]-L-norleucine (0.478 g, 1.04 mmol, 19.3% yield). The ester was a white powder upon lyophilization, whereas the ether was a pale yellow heavy oil.

Part C. Preparation of 6-(benzyloxy)-L-norleucyl-3-[(2,4-dinitrophenyl)amino]-L-alanyl-L-alanyl-L-arginyl-resin.

The product from Part B (0.473 mmol, 0.217 g) was dissolved in 2 ml dimethylformamide. To this solution was added 0.473 mmol (0.064 g) 1-Hydroxy-7-azabenzo-triazole (HOAT, Aldrich, Product 44,545-2), 0.473 mmol (0.180 g) O-(7-Azabenzotriazol-1-yl)-N,N,N′,N′-tetramethyluronium hexafluorophosphate (HATU, Aldrich, Product 44,545-0), and 0.1 mmol of the resin bound product from Example 6, Part B that had been swollen with 2.5 ml CH₂Cl₂ and diluted to 2 ml with dimethylformamide solvent. N,N,-Diopropylethylamine (2.3 mmol, 0.4 ml) (DIEA, Applied Biosystems, Product 400136) was added, the suspension was diluted to 5 ml final volume, and agitated overnight. The resin was washed on a coarse glass scintered filter with dimethylformamide (20 ml×2) alternated with CH₂Cl₂ (20 ml×2) for 2 cycles. Unreacted amino groups remaining on the resin were then capped by dripping a solution through the resin on a coarse glass scintered filter containing 10 ml acetic anhydride (106 mmol), 10 ml N,N,-Diopropylethylamine (57 mmol) and 30 ml dimethylformamide. The resin was washed again as described, and returned to the automated synthesis reaction vessel for Fmoc removal and chain elongation.

Part D. Preparation of L-prolyl-L-leucylglycyl-6-(benzyloxy)-L-norleucyl-3-[(2,4-dinitrophenyl)amino]-L-alanyl-L-alanyl-L-arginyl-resin.

The product from Part C (0.1 mmol) was elongated still attached to the resin using the Applied Biosystems Model 433A Synthesizer and the manufacturer's reagents and reaction vessel designed for the 0.1 mmol scale. The manufacturer's preprogrammed cycles were modified to increase each cycle reaction time by 30 min. The amino acids glycine, L-leucine, and L-proline were added in order, and the amino terminal Fmoc was removed prior to removal of the resin from the machine.

Part E. Preparation of 1-[(7-methoxy-2-oxo-2H-chromen-4-yl)acetyl]-L-prolyl-L-leucylglycyl-6-(benzyloxy)-L-norleucyl-3-[(2,4-dinitrophenyl)amino]-L-alanyl-L-alanyl-L-argininamide (C₅₆H₇₄N₁₄O₁₆).

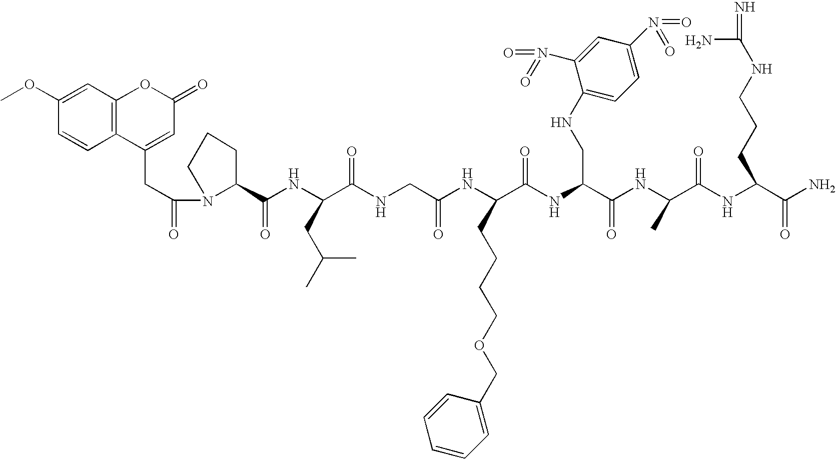

The 7-methoxycoumarin-4-acetic acid reagent (0.234 g, 1.0 mmol) (Aldrich, Product 23,519-9) was suspended in 5 ml dimethylformamide. To the suspension was added 1.0 mmol (0.135 g) 1-hydroxybenzotriazole hydrate (HOBT, Aldrich, Product 15726-0), 1 mmol (0.442 g) (benzotriazol-1-yloxy)tr(dimethylamino)phosphonium hexafluorophosphate (BOP, Castros Reagent, Perseptive Biosystems, Product GEN076503), and 0.25 mmol of the product from Part D that had been swollen with CH₂Cl₂. N,N-Diopropylethylamine (2.3 mmol, 0.4 ml) (DIEA, Applied Biosystems, Product 400136) was added, and the suspension was agitated overnight. The resin was washed on a coarse glass scintered filter with dimethylformamide (20 ml×2) alternated with CH₂Cl₂ (20 ml×2) for 2 cycles. The above protocol was repeated two times to ensure quantitative coupling. The resin was dried under vacuum, and cleaved with 5 ml of a solution of triflouroacetic acid:H₂O:triopropylsilane 18:1:1 for 2-3 hr. The peptide-containing solution was filtered through a glass frit into a total volume of 100 ml of diethylether, and pelleted at 5000 rpm for 5 min. The pellet was washed with ether once, and air dried. The pellet was then dissolved in dimethylsulfoxide, and the peptide was purified on a C18 reverse phase HPLC column developed with a 5-95% acetonitrile gradient in water. Fractions containing >95% pure target peptide substrate were pooled, and lyophilized to dryness. Overall yield was 16% (0.019 g, 0.016 mmol), 98% purity by analytical reversed phase HPLC. Electrospray mass spectrometry gave M+H 1119.5, corresponding to the expected exact mass of 1198.54.

### Example 9

Preparation of 1-[(7-methoxy-2-oxo-2H-chromen-4-yl)acetyl]-L-prolyl-L-leucylglycyl-S-(3-phenylpropyl)-L-cysteinyl-3-[(2,4-dinitrophenyl)amino]-L-alanyl-L-alanyl-L-argininamide.

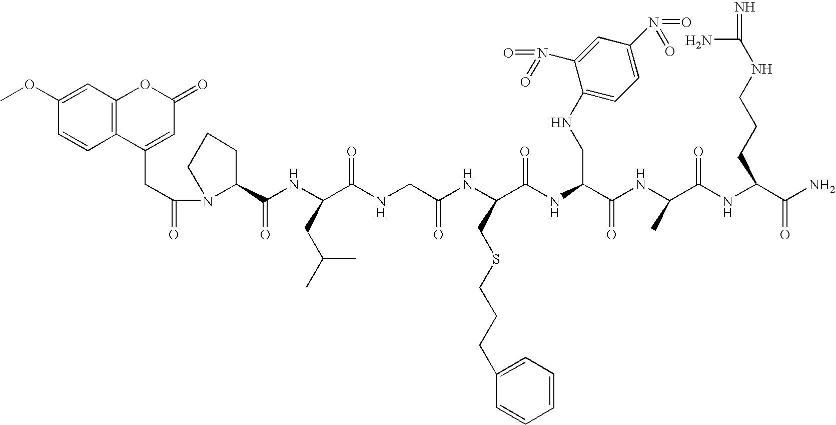

Part A. Synthesis of N-[(9H-fluoren-9-ylmethoxy)carbonyl]-S-(3-phenylpropyl)-L-cysteine.

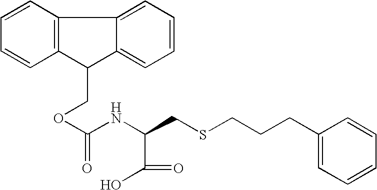

Triopropylsilane (2.5 ml) was added to a solution of N-[(9H-fluoren-9-ylmethoxy) carbonyl]-S-trityl-L-cysteine (3.6 mmolm, 2.1 g) (Aldrich, Product 45,932-1) dissolved in 25 ml trifluoroactic acid, and the vessel was stirred at room temperature for 1 hr to deprotect the cysteine sidechain. The solvent was evaporated under reduced pressure. The residue was partitioned into ethylacetate and 10% Na₂CO₃. The trityl byproduct partitioned into the organic layer and was discarded. 1-Bromo-3-phenylpropane (8 mmol, 1.59 g, 1.2 ml) (Aldrich, B7,740-1) was added to the aqueous mixture, and the reaction was stirred at room temperature under nitrogen over the weekend. The solution was acidified with concentrated HCl to pH 2, and the precipitate was collected on a medium pore glass scintered filter. The residue is extracted with ether, evaporated under reduced pressure, dissolved in methanol, and purified on a C18 reverse phase HPLC column developed with a 5-95% acetonitrile gradient in water. Fractions containing >95% pure target peptide substrate were pooled, and lyophilized to dryness. Overall yield was 8.3% (0.138 g, 0.3 mMoles), 99% purity by analytical reversed phase HPLC. Dulfide formation accounted for the poor yield. Electrospray mass spectrometry gave M+H 462.2 and M+NH₄ 479.2, corresponding to the theoretical exact mass of 461.

Part B. of S-(3-phenylpropyl)-L-cysteinyl-3-[(2,4-dinitrophenyl)amino]-L-alanyl-L-alanyl-L-arginyl-resin.

The product from Part A (0.29 mMole, 0.138 g) was dissolved in 2 ml dimethylformamide. To this solution was added 0.3 mMole (0.041 g)1-Hydroxy-7-azabenzo-triazole (HOAT, Aldrich, Product 44,545-2), 0.3 mMole (0.114 g) O-(7-Azabenzotriazol-1-yl)-N,N,N′,N′-tetramethyluronium hexafluorophosphate (HATU, Aldrich, Product 44,545-0) and 0.1 mMole of the resin bound product from Example 6, Part B that had been swollen with 2.5 ml CH₂Cl₂ and diluted to 2 ml with dimethylformamide solvent. N,N,-Diopropylethylamine (1.1 mMoles, 0.2 ml) (DIEA, Applied Biosystems, Product 400136) was added, the suspension was diluted to 5 ml final volume, and agitated overnight. The resin was washed on a coarse glass scintered filter with dimethylformamide (20 ml×2) alternated with CH₂Cl₂ (20 ml×2) for 2 cycles. Unreacted amino groups remaining on the resin were then capped by dripping a solution through the resin on a coarse glass scintered filter containing 10 ml acetic anhydride (106 mmol), 10 ml N,N,-Diopropylethylamine (57 mmol), and 30 ml dimethylformamide. The resin was washed again as described ,and returned to the automated synthesis reaction vessel for Fmoc removal and chain elongation.

Part C. Preparation of L-prolyl-L-leucylglycyl-S-(3-phenylpropyl)-L-cysteinyl-3-[(2,4-dinitrophenyl)amino]-L-alanyl-L-alanyl-L-arginyl-resin.

The product from Part B (0.1 mmol) was elongated still attached to the resin using the Applied Biosystems Model 433A Synthesizer and the manufacturer's reagents and reaction vessel designed for the 0.1 mmol scale. The manufacturer's preprogrammed cycles were modified to increase each cycle reaction time by 30 min. The amino acids glycine, L-leucine, and L-proline were added in order, and the amino terminal Fmoc was removed prior to removal of the resin from the machine.

Part D. Preparation of 1-[(7-methoxy-2-oxo-2H-chromen-4-yl)acetyl]-L-prolyl-L-leucylglycyl-S-(3-phenylpropyl)-L-cysteinyl-3-[(2,4-dinitrophenyl)amino]-L-alanyl-L-alanyl-L-argininamide (C₅₅H₇₂N₁₄O₁₅S).

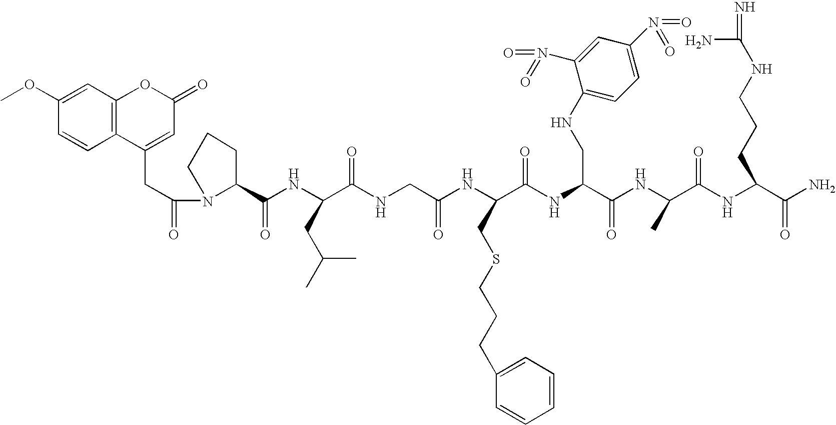

The 7-methoxycoumarin-4-acetic acid reagent (0.117 g, 0.5 mMole) (Aldrich, Product 23,519-9) was suspended in 5 ml dimethylformamide. To the suspension was added 0.5 mmol (0.068 g) 1-hydroxybenzotriazole hydrate (HOBT, Aldrich, Product 15726-0), 0.5 mmol (0.221 g)(benzotriazol-1-yloxy)tr(dimethylamino)phosphonium hexafluorophosphate (BOP, Castros Reagent, Perseptive Biosystems, Product GEN076503), and 0.1 mmol of the product from Part C that had been swollen with CH₂Cl₂. N,N-Diopropylethylamine (1.1 mmol, 0.2 ml) (DIEA, Applied Biosystems, Product 400136) was added, and the suspension was agitated overnight. The resin was washed on a coarse glass scintered filter with dimethylformamide (20 ml×2) alternated with CH₂Cl₂ (20 ml×2) for 2 cycles. The above protocol was repeated to ensure quantitative coupling. The resin was dried under vacuum, and cleaved with 5 ml of a solution of triflouroacetic acid:H₂O:triopropylsilane 18:1:1 for 2-3 hr. The peptide-containing solution was filtered through a glass frit into a total volume of 100 ml of diethylether, and pelleted at 5000 rpm for 5 min. The pellet was washed with ether, and air dried. The pellet was then dissolved in dimethylsulfoxide, and the peptide was purified on a C18 reverse phase HPLC column developed with a 5-95% acetonitrile gradient in water. Fractions containing >95% pure target peptide substrate were pooled, and lyophilized to dryness. Overall yield was 14% (0.019 g, 0.016 mmol), 97% purity by analytical reversed phase HPLC. Electrospray mass spectrometry gave M+H 1201.5, corresponding to the expected exact mass of 1200.5022.

### Example 10

Preparation of of N-2-[(7-methoxy-2-oxo-2H-chromen-4-yl)acetyl]-L-arginyl-L-prolyl-L-leucylglycyl-L-leucyl-3-[(2,4-dinitrophenyl)amino]-L-alanyl-L-alanyl-L-arginyl-L-alpha-glutamyl-D-argininamide.

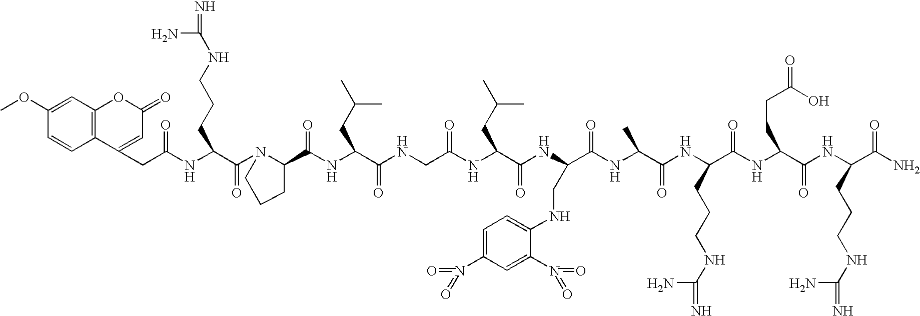

Part A. Preparation of L-alanyl-L-arginyl-L-glutamyl-D-arginyl-resin.

L-alanyl-L-arginyl-L-alpha-glutamyl-D-arginyl-resin was synthesized attached to Applied Biosystems Fmoc-Amide-Resin (Product number 401435) using the Applied Biosystems model 433A Peptide Synthesizer and the manufacturer's reagents and reaction vessel designed for the 0.25 mM synthesis scale. D-Arginine was purchased from Novabiochem (Product 04-13-1045). The manufacturer's preprogrammed cycles were modified to increase each cycle reaction time by 30 min.

Part B. Preparation of 3-[(2,4-dinitrophenyl)aminol-L-alanyl-L-alanyl-L-arginyl-L-alpha-glutamyl-D-arginyl-resin.

The product from Example 1, Part A (1 mmol, 0.492 g) was dissolved in 2 ml dimethylformamide. To this solution was added 1 mmol (0.135 g) 1-Hydroxy-7-azabenzo-triazole (HOAT, Aldrich, Product 44,545-2), 1 mmol (0.380 g) O-(7-Azabenzotriazol-1-yl)-N,N,N′,N′-tetramethyluronium hexafluorophosphate (HATU, Aldrich, Product 44,545-0) and 0.25 mmol of the resin from Part A that had been swollen with 2.5 ml CH₂Cl₂ and diluted to 5 ml with dimethylformamide solvent. N,N,-Diopropylethylamine (4 mmol, 0.7 ml) (DIEA, Applied Biosystems, Product 400136) was added, the suspension was diluted to 10 ml final volume, and agitated overnight. The resin was washed on a coarse glass scintered filter with dimethylformamide (20 ml×2) alternated with CH₂Cl₂ (20 ml×2) for 2 cycles. Unreacted amino groups remaining on the resin were then capped by dripping a solution through the resin on a coarse glass scintered filter containing 10 ml acetic anhydride (106 mmol), 10 ml N,N,-Diopropylethylamine (57 mmol) and 30 ml dimethylformamide. The resin was washed again as described, and returned to the automated synthesis reaction vessel for Fmoc removal and chain elongation.

Part C. Preparation of L-arginyl-L-prolyl-L-leucylglycyl-3-(2-naphthyl)-L-alanyl-3-[(2,4-dinitrophenyl)amino]-L-alanyl-L-alanyl-L-arginyl-L-alpha-glutamyl-D-arginyl-resin.

The product from Part B was elongated still attached to the resin using the Applied Biosystems Model 433A Synthesizer and the manufacturer's reagents and reaction vessel designed for the 0.25 mMole scale. The manufacturer's preprogrammed cycles were modified to increase each cycle reaction time by 30 min. The amino acids L-leucine, glycine, L-leucine, L-proline, and L-arginine were added in order, and the amino terminal Fmoc was removed prior to removal of the resin from the machine.

Part D. Preparation of N-2-[(7-methoxy-2-oxo-2H-chromen-4-yl)acetyl]-L-arginyl-L-prolyl-L-leucylglycyl-L-leucyl-3-[(2,4-dinitrophenyl)amino]-L-alanyl-L-alanyl-L-arginyl-L-alpha-glutamyl-D-argininamide (C₆₆H₉₉N₂₃O₂₀).

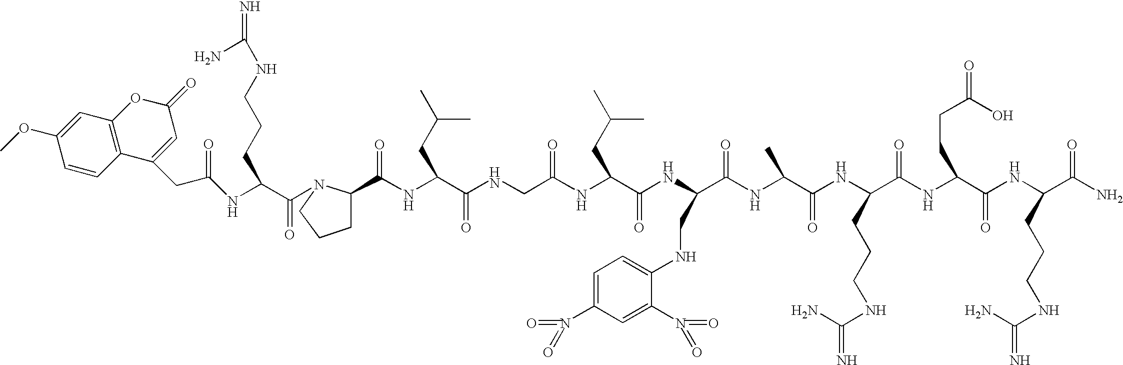

The 7-methoxycoumarin-4-acetic acid reagent (0.117 g, 0.5 mmol) (Aldrich, Product 23,519-9) was suspended in 5 ml dimethylformamide. To the suspension was added 0.5 mmol (0.068 g) 1-hydroxybenzotriazole hydrate (HOBT, Aldrich, Product 15726-0), 0.5 mmol (0.221 g) (benzotriazol-1-yloxy)tr(dimethylamino)phosphonium hexafluorophosphate (BOP, Castros Reagent, Perseptive Biosystems, Product GEN076503) and 0.25 mmol of the product from Part C that had been swollen with CH₂Cl₂. N,N-Diopropylethylamine (1 mmol, 0.174 ml) (DIEA, Applied Biosystems, Product 400136) was added in two portions, and the suspension was agitated overnight. The resin was washed on a coarse glass scintered filter with dimethylformamide (20 ml×2) alternated with CH₂Cl₂ (20 ml×2) for 2 cycles. The above protocol is repeated to ensure quantitative coupling. The resin was dried under vacuum, and cleaved with 5 ml of a solution of triflouroacetic acid:H₂O:triopropylsilane 18:1:1 for 2-3 hr. The peptide-containing solution was filtered through a glass frit into a total volume of 100 ml of diethylether, and pelleted at 5000 rpm for 5 min. The pellet was washed with ether, and air dried. The pellet was then dissolved in dimethylsulfoxide, and the peptide was purified on a C18 reverse phase HPLC column developed with a 5-95% acetonitrile gradient in water. Fractions containing >95% pure target peptide substrate were pooled, and lyophilized to dryness. Overall yield was 70% (0.270 g, 0.176 mmol), 98% purity by analytical reversed phase HPLC. Electrospray mass spectrometry gave an M+2H 768.7, corresponding to the expected exact mass of 1533.7437.

### Example 11

Preparation of N-2-[(7-methoxy-2-oxo-2H-chromen-4-yl)acetyl]-L-arginyl-L-prolyl-L-leucylglycyl-L-leucyl-3-[(2,4-dinitrophenyl)amino]-L-alanyl-L-alanyl-D-arginyl-D-alpha-glutamyl-D-argininamide.

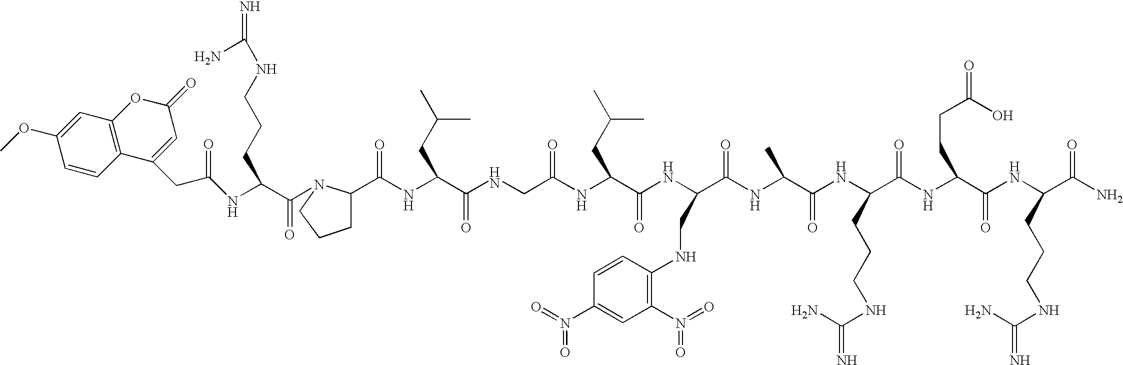

Part A. Preparation of L-alanyl-D-arginyl-D-alpha-glutamyl-D-arginyl-resin.

L-alanyl-D-arginyl-D-alpha-glutamyl-D-arginyl-resin was synthesized attached to Applied Biosystems Fmoc-Amide-Resin (Product number 401435) using the Applied Biosystems model 433A Peptide Synthesizer and the manufacturer's reagents and reaction vessel designed for the 0.25 mM synthesis scale. D-Arginine (Product 04-13-1045) and D-glutamic acid (Product 04-13-1051) were purchased from Novabiochem. The manufacturer's preprogrammed cycles were modified to increase each cycle reaction time by 30 min.

Part B. Preparation of 3-1(2,4-dinitrophenyl)aminol-L-alanyl-L-alanyl-D-arginyl-D-alpha-glutamyl-D-arginyl-resin.

The product from Example 1, Part A (1 mmol, 0.492 g) was dissolved in 2 ml dimethylformamide. To this solution wer added 1 mmol (0.135 g) 1-Hydroxy-7-azabenzo-triazole (HOAT, Aldrich, Product 44,545-2), 1 mmol (0.380 g) O-(7-Azabenzotriazol-1-yl)-N,N,N′,N′-tetramethyluronium hexafluorophosphate (HATU, Aldrich, Product 44,545-0) and 0.25 mmol of the product from Part A that had been swollen with 2.5 ml CH₂Cl₂ and diluted to 5 ml with dimethylformamide solvent. N,N,-Diopropylethylamine (4 mmol, 0.7 ml) (DIEA, Applied Biosystems, Product 400136) was added, the suspension was diluted to 10 ml final volume, and agitated overnight. The resin was washed on a coarse glass scintered filter with dimethylformamide (20 ml×2) alternated with CH₂Cl₂ (20 ml×2) for 2 cycles. Unreacted amino groups remaining on the resin were then capped by dripping a solution through the resin on a coarse glass scintered filter containing 10 ml acetic anhydride (106 mmol), 10 ml N,N,-diopropylethylamine (57 mmol) and 30 ml dimethylformamide. The resin was washed again as described, and returned to the automated synthesis reaction vessel for Fmoc removal and chain elongation.

Part C. Preparation of L-arginyl-L-prolyl-L-leucylglycyl-L-leucyl-3-[(2,4-dinitrophenyl)amino]-L-alanyl-L-alanyl-D-arginyl-D-alpha-glutamyl-D-arginyl-resin.

The product from Part B was elongated still attached to the resin using the Applied Biosystems Model 433A Synthesizer and the manufacturer's reagents and reaction vessel designed for the 0.25 mMole scale. The manufacturer's preprogrammed cycles were modified to increase each cycle reaction time by 30 min. The amino acids L-leucine, glycine, L-leucine, L-proline, and L-arginine were added in order, and the amino-terminal Fmoc was removed prior to removal of the resin from the machine.

Part D. Preparation of N-2-[(7-methoxy-2-oxo-2H-chromen-4-yl)acetyl]-L-arginyl-L-prolyl-L-leucylglycyl-L-leucyl-3-[(2,4-dinitrophenyl)aminol-L-alanyl-L-alanyl-D-arginyl-D-alpha-glutamyl-D-argininamide (C₆₆H₉₉N₂₃O₂₀).

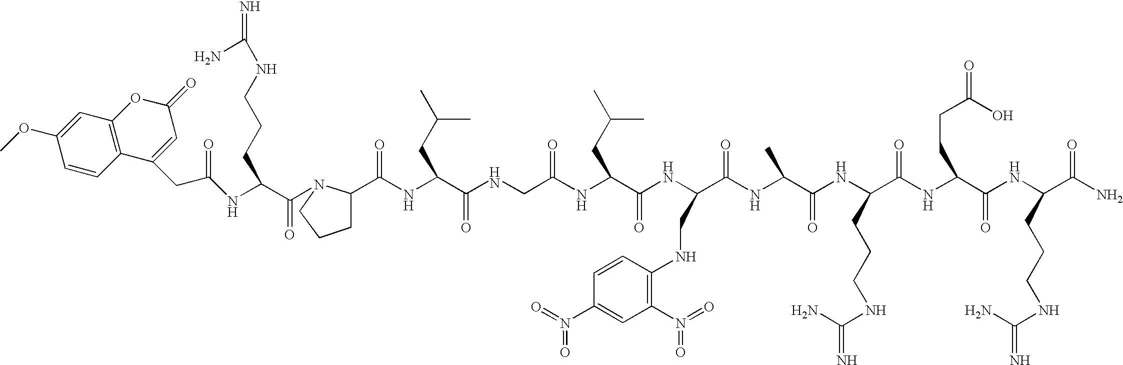

The 7-methoxycoumarin-4-acetic acid reagent (0.117 g, 0.5 mmol) (Aldrich, Product 23,519-9) was suspended in 5 ml dimethylformamide. To the suspension were added 0.5 mmol (0.068 g) 1-hydroxybenzotriazole hydrate (HOBT, Aldrich, Product 15726-0), 0.5 mmol (0.221 g) (benzotriazol-1-yloxy)tr(dimethylamino)phosphonium hexafluorophosphate (BOP, Castros Reagent, Perseptive Biosystems, Product GEN076503), and 0.25 mmol of the product from Part C that had been swollen with CH₂Cl₂. N,N-diopropylethylamine (1 mmol, 0.174 ml) (DIEA, Applied Biosystems, Product 400136) was added in two portions, and the suspension was agitated overnight. The resin was washed on a coarse glass scintered filter with dimethylformamide (20 ml×2) alternated with CH₂Cl₂ (20 ml×2) for 2 cycles. The above protocol was repeated to ensure quantitative coupling. The resin was washed on a coarse glass scintered filter with dimethylformamide (20 ml×2) alternated with CH₂Cl₂ (20 ml×2) for 2 cycles. The resin was dried under vacuum, and cleaved with 5 ml of a solution of triflouroacetic acid:H₂O:triopropylsilane 18:1:1 for 2-3 hr. The peptide-containing solution was filtered through a glass frit into a total volume of 100 ml of diethylether, and pelleted at 5000 rpm for 5 min. The pellet was washed with ether, and air dried. The pellet was then dissolved in dimethylsulfoxide, and the peptide was purified on a C18 reverse phase HPLC column developed with a 5-95% acetonitrile gradient in water. Fractions containing >95% pure target peptide substrate were pooled, and lyophilized to dryness. Overall yield was 70% (0.270 g, 0.176 mmol), 98% purity by analytical reversed phase HPLC. Electrospray mass spectrometry gives an M+2H 768.9 and M+3H 513, corresponding to the expected exact mass of 1533.7437.

### Example 12

Preparation of N-2-[(7-methoxy-2-oxo-2H-chromen-4-yl)acetyl]-D-arginyl-L-prolyl-L-leucylglyeyl-L-leucyl-3-[(2,4-dinitrophenyl)amino]-L-alanyl-L-alanyl-L-arginyl-D-alpha-glutamyl-D-argininamide.

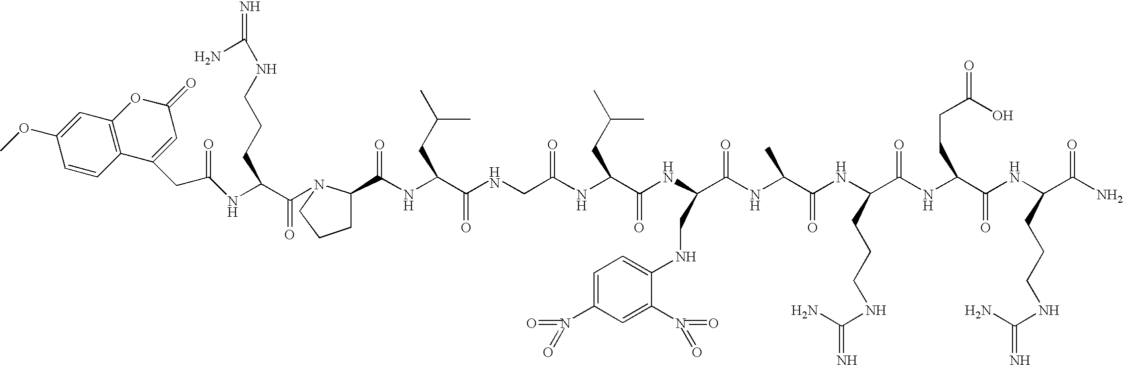

Part A. Preparation of L-alanyl-L-arginyl-D-alpha-glutamyl-D-arginyl-resin.

L-alanyl-L-arginyl-D-alpha-glutamyl-D-arginyl-resin was synthesized attached to Applied Biosystems Fmoc-Amide-Resin (Product number 401435) using the Applied Biosystems model 433A Peptide Synthesizer and the manufacturer's reagents and reaction vessel designed for the 0.25 mM synthesis scale. D-Arginine (Product 04-13-1045) and D-glutamic acid (Product 04-13-1051) were purchased from Novabiochem. The manufacturer's preprogrammed cycles were modified to increase each cycle reaction time by 30 min.

Part B. Preparation of 3-[(2,4-dinitrophenyl)amino]-L-alanyl-L-alanyl-L-arginyl-D-alpha-glutamyl-D-arginyl-resin.

The product from Example 1, Part A (1 mmol, 0.492 g) is dissolved in 2 ml dimethylformamide. To this solution were added 1 mmol (0.135 g) 1-Hydroxy-7-azabenzo-triazole (HOAT, Aldrich, Product 44,545-2), 1 mmol (0.380 g) O-(7-Azabenzotriazol-1-yl)-N,N,N′,N′-tetramethyluronium hexafluorophosphate (HATU, Aldrich, Product 44,545-0), and 0.25 mmol of the product from Part A that had been swollen with 2.5 ml CH₂Cl₂ and diluted to 5 ml with dimethylformamide solvent. N,N,-diopropylethylamine (4 mmol, 0.7 ml) (DIEA, Applied Biosystems, Product 400136) was added, the suspension was diluted to 10 ml final volume, and agitated overnight. The resin was washed on a coarse glass scintered filter with dimethylformamide (20 ml×2) alternated with CH₂Cl₂ (20 ml×2) for 2 cycles. Unreacted amino groups remaining on the resin were then capped by dripping a solution through the resin on a coarse glass scintered filter containing 10 ml acetic anhydride (106 mmol), 10 ml N,N,-diopropylethylamine (57 mmol) and 30 ml dimethylformamide. The resin was washed again as described, and returned to the automated synthesis reaction vessel for Fmoc removal and chain elongation.

Part C. Preparation of D-arginyl-L-prolyl-L-leucylglycyl-3-(2-naphthyl)-L-alanyl-3-[(2,4-dinitrophenyl)amino]-L-alanyl-L-alanyl-L-arginyl-D-alpha-glutamyl-D-arginyl-resin.

The product from Part B was elongated still attached to the resin using the Applied Biosystems Model 433A Synthesizer and the manufacturer's reagents and reaction vessel designed for the 0.25 mmol scale. The manufacturer's preprogrammed cycles were modified to increase each cycle reaction time by 30 min. L-leucine, glycine, L-leucine, L-proline, and D-arginine (Novabiochem, Product 04-13-1045) were added in order, and the amino-terminal Fmoc was removed prior to removal of the resin from the machine.

Part D. Preparation of N-2-[(7-methoxy-2-oxo-2H-chromen-4-yl)acetyl]-D-arginyl-L-prolyl-L-leucylglycyl-L-leucyl-3-[(2,4-dinitrophenyl)amino]-L-alanyl-L-alanyl-L-arginyl-D-alpha-glutamyl-D-argininamide (C₆₆H₉₉N₂₃O₂₀).

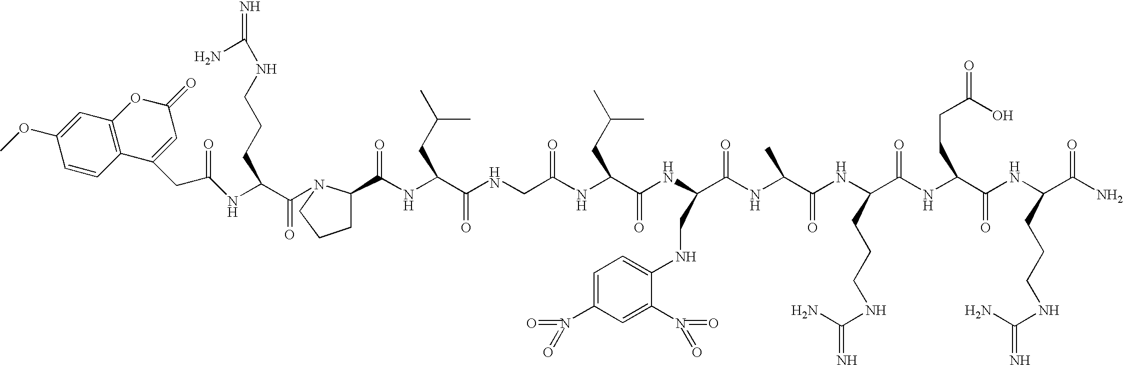

The 7-methoxycoumarin-4-acetic acid reagent (0.117 g, 0.5 mmol) (Aldrich, Product 23,519-9) was suspended in 5 ml dimethylformamide. To the suspension were added 0.5 mmol (0.068 g) 1-hydroxybenzotriazole hydrate (HOBT, Aldrich, Product 15726-0), 0.5 mmol (0.221 g) (benzotriazol-1-yloxy)tr(dimethylamino)phosphonium hexafluorophosphate (BOP, Castros Reagent, Perseptive Biosystems, Product GEN076503) and 0.25 mmol of the product from Part C that had been swollen with CH₂Cl₂. N,N-Diopropylethylamine (1 mmol, 0.174 ml) (DIEA, Applied Biosystems, Product 400136) was added in two portions, and the suspension was agitated overnight. The resin was washed on a coarse glass scintered filter with dimethylformamide (20 ml×2) alternated with CH₂Cl₂ (20 ml×2) for 2 cycles. The above protocol was repeated to ensure quantitative coupling. The resin was dried under vacuum, and cleaved with 5 ml of a solution of triflouroacetic acid:H₂O:triopropylsilane 18:1:1 for 2-3 hr. The peptide-containing solution was filtered through a glass frit into a total volume of 100 ml of diethyl ether, and pelleted at 5000 rpm for 5 min. The pellet was washed with ether, and air dried. The pellet was then dissolved in dimethylsulfoxide, and the peptide was purified on a C18 reverse phase HPLC column developed with a 5-95% acetonitrile gradient in water. Fractions containing >95% pure target peptide substrate were pooled, and lyophilized to dryness. Overall yield was 70% (0.270 g, 0.176 mmol), 98% purity by analytical reversed phase HPLC. Electrospray mass spectrometry gave M+2H 768.7, corresponding to the expected exact mass of 1533.7437.

### Example 13

Preparation of N-2-[(7-methoxy-2-oxo-2H-chromen-4-yl)acetyl]-D-arginyl-L-prolyl-L-leucylglycyl-L-leucyl-3-[(2,4-dinitrophenyl)amino]-L-alanyl-L-alanyl-D-arginyl-D-alpha-glutamyl-D-argininamide (C₆₆H₉₉N₂₃O₂₀).

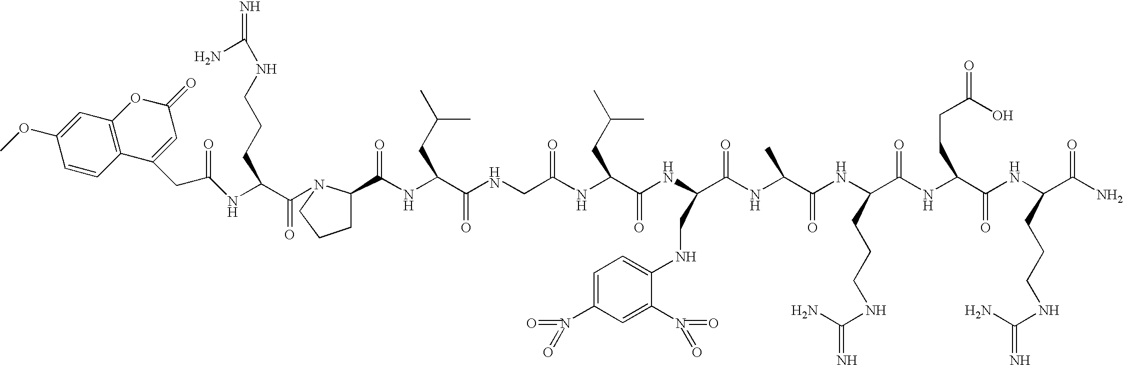

Part A. Preparation of D-arginyl-L-prolyl-L-leucylglycyl-L-leucyl-3-[(2,4-dinitrophenyl)amino]-L-alanyl-L-alanyl-D-arginyl-D-alpha-glutamyl-D-arginyl-resin.

The product from Example 11, Part B was elongated still attached to the resin using the Applied Biosystems Model 433A Synthesizer and the manufacturer's reagents and reaction vessel designed for the 0.25 mmol scale. The manufacturer's pre-programmed cycles were modified to increase each cycle reaction time by 30 min. L-leucine, glycine, L-leucine, L-proline, and D-arginine (Novabiochem, Product 04-13-1045) were added in order, and the amino-terminal Fmoc was removed prior to removal of the resin from the machine.

Part B. Preparation of N-2-[(7-methoxy-2-oxo-2H-chromen-4-yl)acetyl]-D-arginyl-L-prolyl-L-leucylglycyl-L-leucyl-3-[(2,4-dinitrophenyl)amino]-L-alanyl-L-alanyl-D-arginyl-D-alpha-glutamyl-D-argininamide (C₆₆H₉₉N₂₃O₂₀).

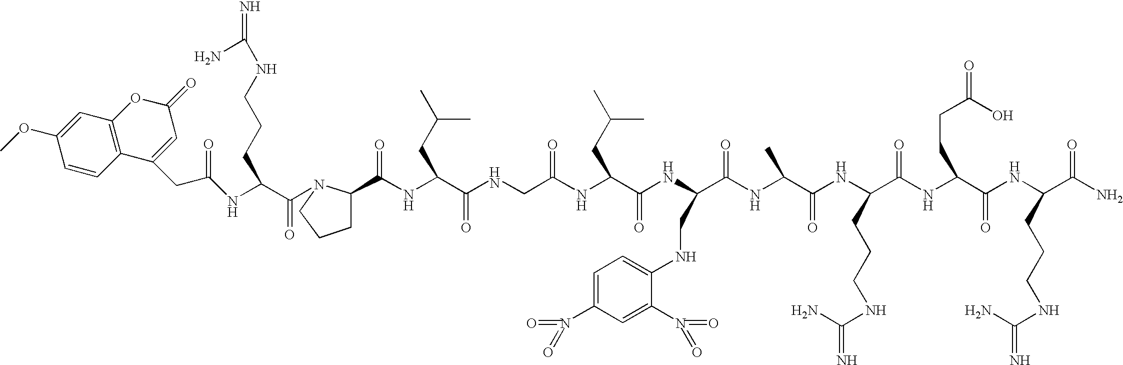

The 7-methoxycoumarin-4-acetic acid reagent (0.234 g, 1.0 mmol) (Aldrich, Product 23,519-9) was suspended in 5 ml dimethylformamide. To the suspension were added 1.0 mmol (0.136 g) 1-hydroxybenzotriazole hydrate (HOBT, Aldrich, Product 15726-0), 1.0 mmol (0.442 g) (benzotriazol-1-yloxy)tr(dimethylamino)phosphonium hexafluorophosphate (BOP, Castros Reagent, Perseptive Biosystems, Product GEN076503) and 0.25 mmol of the product from Part C that had been swollen with CH₂Cl₂. N,N-Diopropylethylamine (2 mmol, 0.350 ml) (DIEA, Applied Biosystems, Product 400136) was added in two portions, and the suspension is agitated overnight. The resin was washed on a coarse glass scintered filter with dimethylformamide (20 ml×2) alternated with CH₂Cl₂ (20 ml×2) for 2 cycles. The above protocol was repeated to ensure quantitative coupling. The resin was dried under vacuum, and cleaved with 5 ml of a solution of triflouroacetic acid:H₂O:triopropylsilane 18:1:1 for 2-3 hr. The peptide-containing solution was filtered through a glass frit into a total volume of 100 ml of diethylether, and pelleted at 5000 rpm for 5 min. The pellet was washed with ether, and air dried. The pellet was then dissolved in dimethylsulfoxide, and the peptide was purified on a C18 reverse phase HPLC column developed with a 5-95% acetonitrile gradient in water. Fractions containing >95% pure target peptide substrate were pooled, and lyophilized to dryness. Overall yield was 56% (0.213 g, 0.139 mmol), 98% purity by analytical reversed phase HPLC. Electrospray mass spectrometry gave M+2H 768.7, corresponding to the expected exact mass of 1533.7437.

### Example 14

Preparation of N-2-[(7-methoxy-2-oxo-2H-chromen-4-yl)acetyl]-D-arginyl-L-prolyl-L-glutaminylglycyl-L-leucyl-3-[(2,4-dinitrophenyl)amino]-L-alanyl-L-alanyl-D-arginyl-D-alpha-glutamyl-D-argininamide.

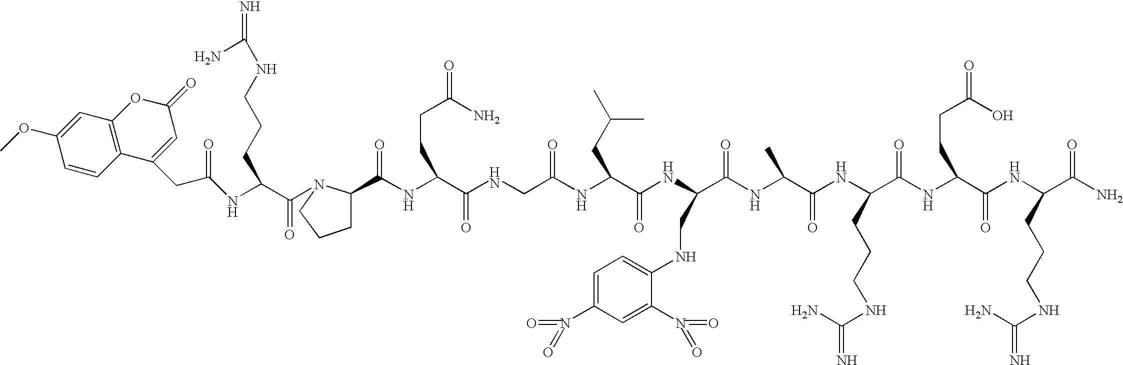

Part A. Preparation of D-arginyl-L-prolyl-L-glutaminylglycyl-L-leucyl-3-[(2,4-dinitrophenyl)amino]-L-alanyl-L-alanyl-D-arginyl-D-alpha-glutamyl-D-arginyl-resin.

The product from Example 11, Part B was elongated still attached to the resin using the Applied Biosystems Model 433A Synthesizer and the manufacturer's reagents and reaction vessel designed for the 0.25 mMole scale. The manufacturer's preprogrammed cycles are modified to increase each cycle reaction time by 30 min. L-leucine, glycine, L-glutamine, L-proline, and D-arginine (Novabiochem, Product 04-13-1045) were added in order, and the amino-terminal Fmoc was removed prior to removal of the resin from the machine.

Part B. Preparation of N-2-[(7-methoxy-2-oxo-2H-chromen-4-yl)acetyl]-D-arginyl-L-prolyl-L-glutaminylglycyl-L-leucyl-3-[(2,4-dinitrophenyl)aminol-L-alanyl-L-alanyl-D-arginyl-D-alpha-glutamyl-D-argininamide (C₆₅H₉₆N₂₄O₂₁).

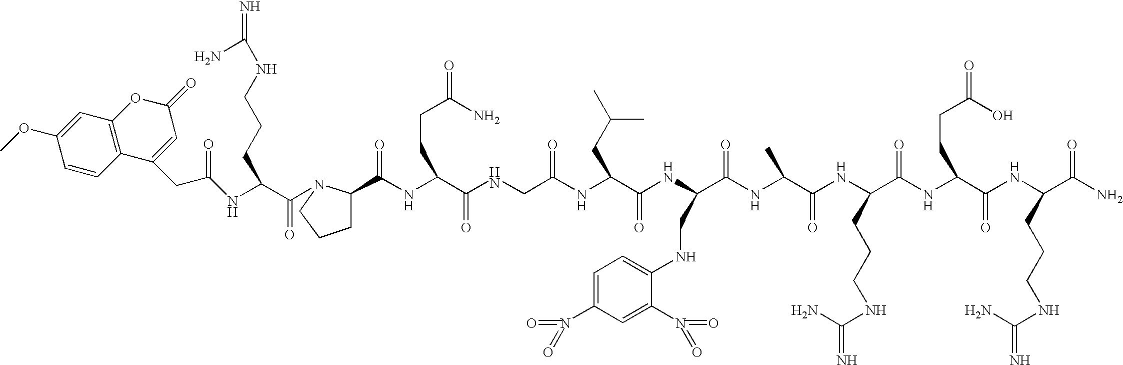

The 7-methoxycoumarin-4-acetic acid reagent (0.234 g, 1.0 mmol) (Aldrich, Product 23,519-9) is suspended in 5 ml dimethylformamide. To the suspension were added 1.0 mmol (0.136 g) 1-hydroxybenzotriazole hydrate (HOBT, Aldrich, Product 15726-0), 1.0 mmol (0.442 g) (benzotriazol-1-yloxy)tr(dimethylamino)phosphonium hexafluorophosphate (BOP, Castros Reagent, Perseptive Biosystems, Product GEN076503) and 0.25 mmol of product from Part C that had been swollen with CH₂Cl₂. N,N-Diopropylethylamine (2 mmol, 0.350 ml) (DIEA, Applied Biosystems, Product 400136) was added in two portions, and the suspension is agitated overnight. The resin was washed on a coarse glass scintered filter with dimethylformamide (20 ml×2) alternated with CH₂Cl₂ (20 ml×2) for 2 cycles. The above protocol was repeated to ensure quantitative coupling. The resin was dried under vacuum, and cleaved with 5 ml of a solution of triflouroacetic acid:H₂O:triopropylsilane 18:1:1 for 2-3 hr. The peptide-containing solution was filtered through a glass frit into a total volume of 100 ml of diethylether, and pelleted at 5000 rpm for 5 min. The pellet was washed with ether, and air dried. The pellet was then dissolved in dimethylsulfoxide, and the peptide was purified on a C18 reverse phase HPLC column developed with a 5-95% acetonitrile gradient in water. Fractions containing >95% pure target peptide substrate were pooled, and lyophilized to dryness. Overall yield was 35% (0.135 g, 0.139 mmol), 97% purity by analytical reversed phase HPLC. Electrospray mass spectrometry gave M+2H 775.4, M+2Na 797.6, M+H+Na 786.4, M+3H 517.5, and M+2H+Na 524.8, corresponding to the expected exact mass of 1548.7182.

### Example 15

Preparation of N-2-1(7-methoxy-2-oxo-2H-chromen-4-yl)acetyl]-D-lysyl-L-prolyl-L-leucylglycyl-L-leucyl-3-[(2,4-dinitrophenyl)amino]-L-alanyl-L-alanyl-D-arginyl-D-alpha-glutamyl-D-argininamide.

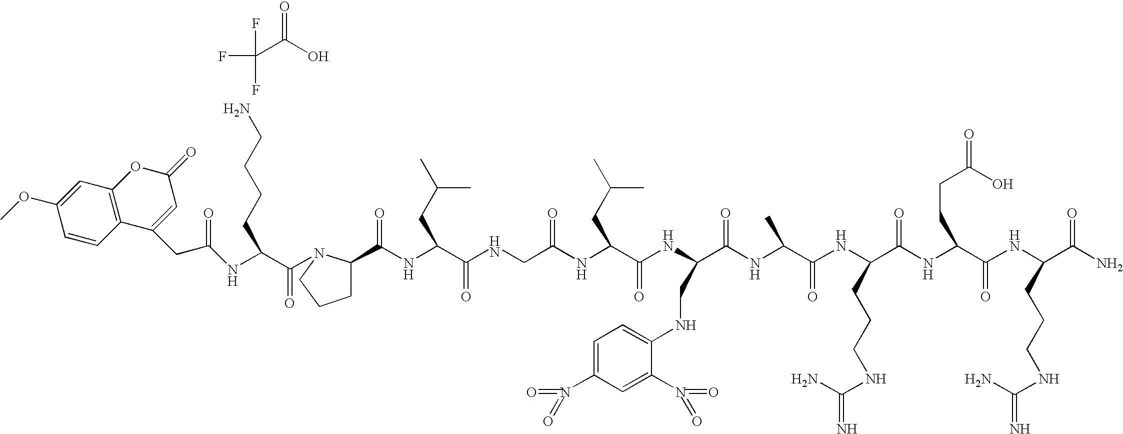

Part A. Preparation of D-lysyl-L-prolyl-L-leucylglycyl-L-leucyl-3-[(2,4-dinitrophenyl)amino]-L-alanyl-L-alanyl-D-arginyl-D-alpha-glutamyl-D-arginyl-resin.

The product from Example 11, Part B (0.25 mmol) was elongated still attached to the resin using the Applied Biosystems Model 433A Synthesizer and the manufacturer's reagents and reaction vessel designed for the 0.25 mmol scale. The manufacturer's preprogrammed cycles were modified to increase each cycle reaction time by 30 min. L-leucine, glycine, L-leucine, L-proline, and D-lysine (Novabiochem, Product 04-13-1026) were added in order, and the amino-terminal Fmoc was removed prior to removal of the resin from the machine.

Part B. Preparation of N-2-[(7-methoxy-2-oxo-2H-chromen-4-yl)acetyl]-D-lysyl-L-prolyl-L-leucylglycyl-L-leucyl-3-[(2,4-dinitrophenyl)amino]-L-alanyl-L-alanyl-D-arginyl-D-alpha-glutamyl-D-argininamide (C₆₆H₉₉N₂₁O₂₀.C₂HF₃O).

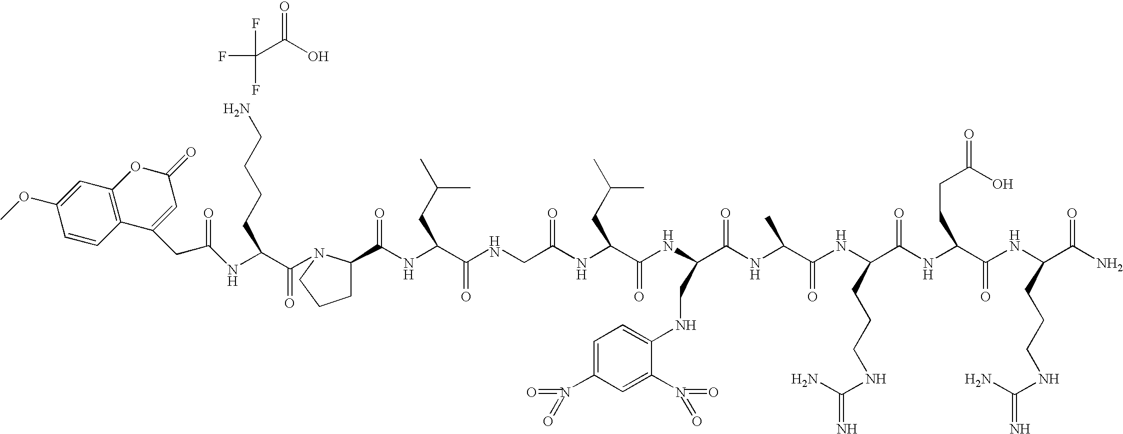

The 7-methoxycoumarin-4-acetic acid reagent (0.234 g, 1.0 mmol) (Aldrich, Product 23,519-9) was suspended in 5 ml dimethylformamide. To the suspension were added 1.0 mmol (0.136 g) 1-hydroxybenzotriazole hydrate (HOBT, Aldrich, Product 15726-0), 1.0 mmol (0.442 g) (benzotriazol-1-yloxy)tr(dimethylamino)phosphonium hexafluorophosphate (BOP, Castros Reagent, Perseptive Biosystems, Product GEN076503), and 0.25 mmol of product from Part C that had been swollen with CH₂Cl₂. N,N-Diopropylethylamine (2 mmol, 0.350 ml) (DIEA, Applied Biosystems, Product 400136) was added in two portions, and the suspension is agitated overnight. The resin was washed on a coarse glass scintered filter with dimethylformamide (20 ml×2) alternated with CH₂Cl₂ (20 ml×2) for 2 cycles. The above protocol is repeated to ensure quantitative coupling. The resin was dried under vacuum, and cleaved with 5 ml of a solution of triflouroacetic acid:H₂O:triopropylsilane 18:1:1 for 2-3 hr. The peptide-containing solution was filtered through a glass frit into a total volume of 100 ml of diethylether, and pelleted at 5000 rpm for 5 min. The pellet was washed with ether, and air dried. The pellet was then dissolved in dimethylsulfoxide, and the peptide was purified on a C18 reverse phase HPLC column developed with a 5-95% acetonitrile gradient in water. Fractions containing >95% pure target peptide substrate were pooled, and lyophilized to dryness. Overall yield was 53% (0.214 g, 0.132 mmol), 98% purity by analytical reversed phase HPLC. Electrospray mass spectrometry gave M+H+Na+TFA 820.6, corresponding to the expected exact mass of 1505.7375.

### Example 16

Preparation of N-2-[(7-methoxy-2-oxo-2H-chromen-4-yl)acetyl]-N-6-[6-({5-[(4S)-2-oxohexahydro-1H-thieno[3,4-d]imidazol-4-yl]pentanoyl}amino)hexanoyl]-D-lysyl-L-prolyl-L-leucylglycyl-L-leucyl-3-[(2,4-dinitrophenyl)amino]-L-alanyl-L-alanyl-D-arginyl-D-alpha-glutamyl-D-arginamide.

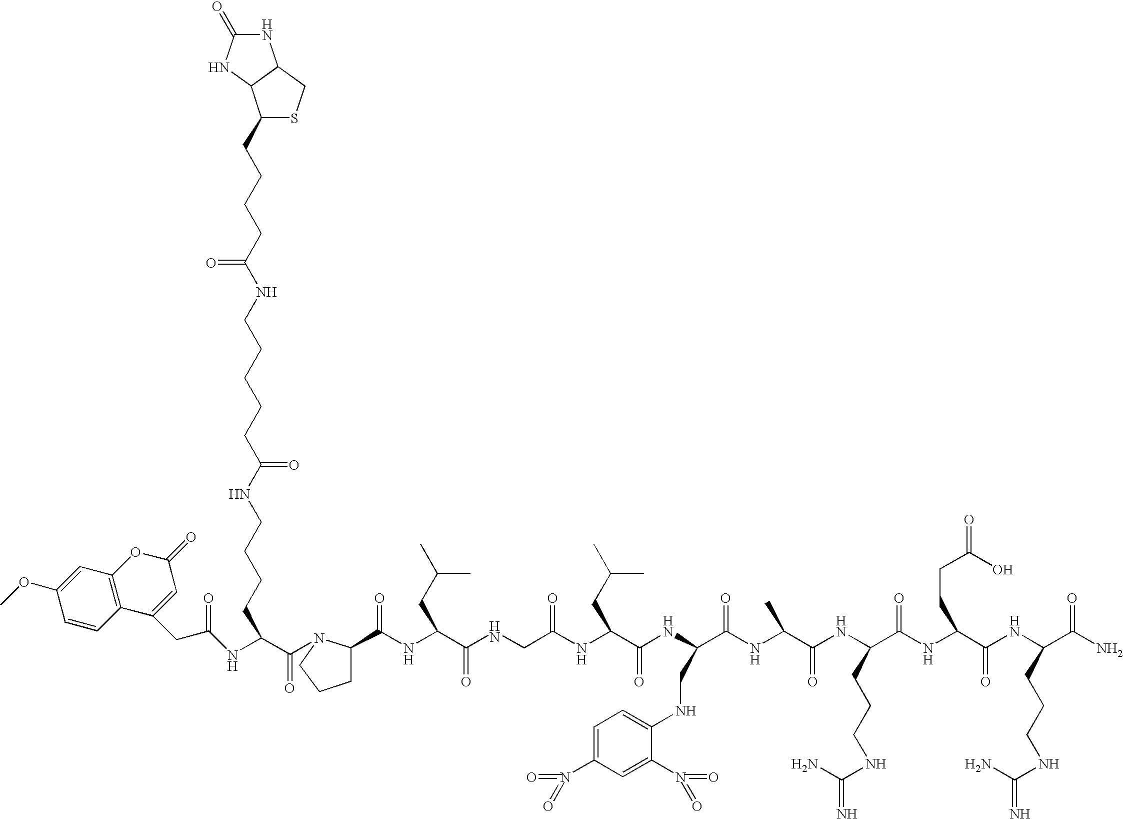

Part A. Preparation of D-lysyl-L-prolyl-L-leucylglycyl-L-leucyl-3-[(2,4-dinitrophenyl)amino]-L-alanyl-L-alanyl-D-arginyl-D-alpha-glutamyl-D-arginyl-resin.

The product from Example 11, Part B (0.25 mmol) was elongated still attached to the resin using the Applied Biosystems Model 433A Synthesizer and the manufacturer's reagents and reaction vessel designed for the 0.25 mmol scale. The manufacturer's preprogrammed cycles were modified to increase each cycle reaction time by 30 min. L-leucine, glycine, L-leucine, L-proline, and D-lysine were added in order, and the amino-terminal Fmoc was removed prior to removal of the resin from the machine. However, to enable selective sidechain deprotection, N-alpha-Fmoc-N-epsilon-4-methyltrityl-D-lysine was (Bachem, Product B-2620).

Part B. Preparation of N-2-[(7-methoxy-2-oxo-2H-chromen-4-yl)acetyl]-D-lysyl-L-prolyl-L-leucylglycyl-L-leucyl-3-[(2,4-dinitrophenyl)amino]-L-alanyl-L-alanyl-D-arginyl-D-alpha-glutamyl-D-arginyl-resin.

The 7-methoxycoumarin-4-acetic acid reagent (0.234 g, 1.0 mmol) (Aldrich, Product 23,519-9) was suspended in 5 ml dimethylformamide. To the suspension were added 1.0 mmol (0.136 g) 1-hydroxybenzotriazole hydrate (HOBT, Aldrich, Product 15726-0), 1.0 mmol (0.442 g) (benzotriazol-1-yloxy)tr(dimethylamino)phosphonium hexafluorophosphate (BOP, Castros Reagent, Perseptive Biosystems, Product GEN076503) and 0.25 mmol of the product from Part C that had been swollen with CH₂Cl₂. N,N-Diopropylethylamine (2 mmol, 0.350 ml) (DIEA, Applied Biosystems, Product 400136) was added in two portions, and the suspension was agitated overnight. The resin was washed on a coarse glass scintered filter with dimethylformamide (20 ml×2) alternated with CH₂Cl₂ (20 ml×2) for 2 cycles. The above protocol was repeated to ensure quantitative coupling.

Part C. Preparation of N-6-(6-aminohexanoyl)-N-2-[(7-methoxy-2-oxo-2H-chromen-4-yl)acetyl]-D-lysyl-L-prolyl-L-leucylglycyl-L-leucyl-3-[(2,4-dinitrophenyl)amino]-L-alanyl-L-alanyl-D-arginyl-D-alpha-glutamyl-D-arginyl-resin.

A solution containing 2% triflouroacetic acid and 5% triopropylsilane in CH₂Cl₂ (100 ml) was passed through the resin from Part B in order to selectively deprotect the lysine sidechain until the fractions eluting from the resin were no longer yellow. Resin was washed with CH₂Cl₂ (20 ml×2) followed by dimethylformamide (20 ml×2) for two cycles. 6-{[(9H-fluoren-9-ylmethoxy)carbonyl]amino}hexanoic acid (1 mmol, 0.353 g) was dissolved in 2 ml dimethylformamide. To this solution were added 1 mmol 1-Hydroxy-7-azabenzo-triazole (HOAT, Aldrich, Product 44,545-2), 1 mmol O-(7-Azabenzotriazol-1-yl)-N,N,N′,N′-tetramethyluronium hexafluorophosphate (HATU, Aldrich, Product 44,545-0), and 0.25 mmol of the product from Part B that had been swollen with 2.5 ml CH₂Cl₂ and diluted to 5 ml with dimethylformamide solvent. N,N,-Diopropylethylamine (4 mmol, 0.7 ml) (DIEA, Applied Biosystems, Product 400136) was added, the suspension was diluted to 10 ml final volume, and agitated overnight. The resin was washed on a coarse glass scintered filter with dimethylformamide (20 ml×2) alternated with CH₂Cl₂ (20 ml×2) for 2 cycles. The Fmoc group was removed using the Applied Biosystems Model 433A Synthesizer standard deprotection cycle and the manufacturer's reagents and reaction vessel designed for the 0.25 mmol scale.

Part D. Preparation of N-2-[(7-methoxy-2-oxo-2H-chromen-4-yl)acetyl]-N-6-[6-({5-[(4S)-2-oxohexahydro-1H-thieno[3,4-d]imidazol-4-yl]pentanoyl}amino)hexanoyl]-D-lysyl-L-prolyl-L-leucylglycyl-L-leucyl-3-[(2,4-dinitrophenyl)amino]-L-alanyl-L-alanyl-D-arginyl-D-alpha-glutamyl-D-arginamide.

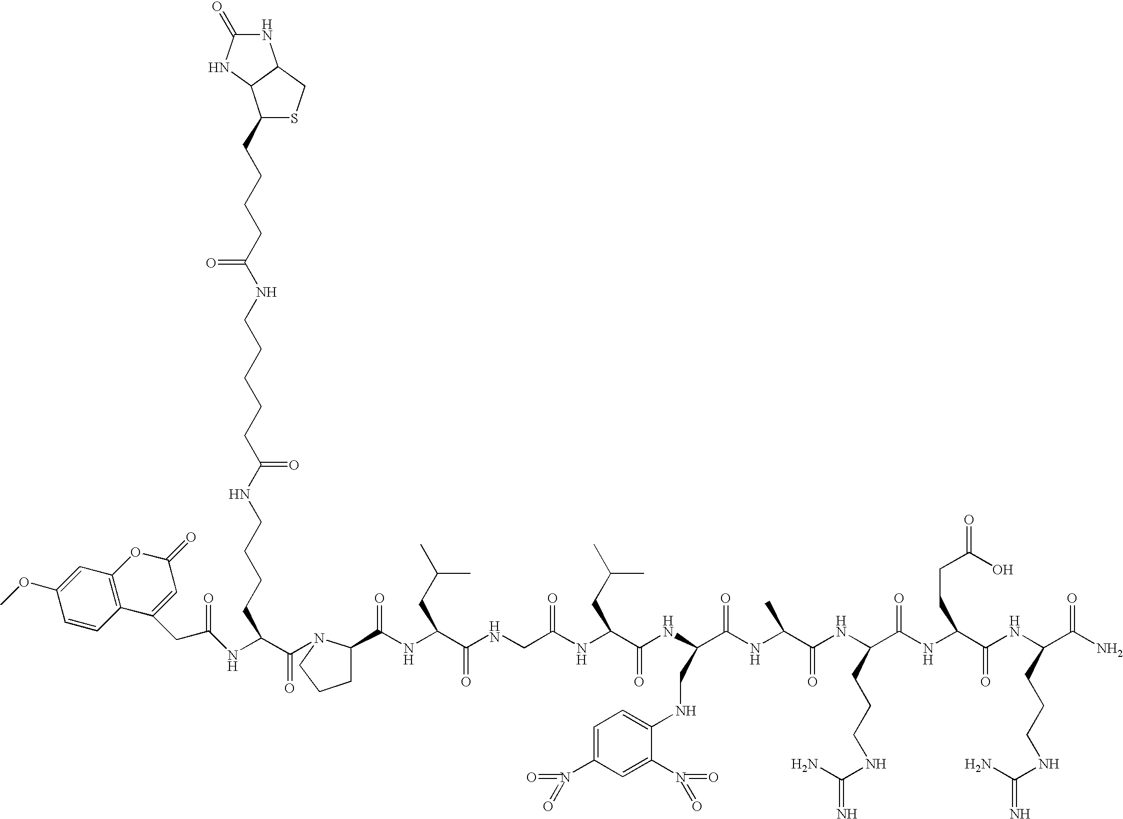

5-[(3aS ,4S,6aR)-2-oxohexahydro-1H-thieno[3,4-d]imidazol-4-yl]pentanoic acid (1 mmol, 0.244 g) was dissolved in 10 ml dimethylformamide. To this solution were added 1 mmol (0.135 g) 1-hydroxybenzotriazole hydrate (HOBT, Aldrich, Product 15726-0), 1 mmol (0.442 g) (benzotriazol-1-yloxy)tr(dimethylamino)phosphonium hexafluorophosphate (BOP, Castros Reagent, Perseptive Biosystems, Product GEN076503) and 0.25 mmol of the product from Part C that had been swollen with CH₂Cl₂. N,N-Diopropylethylamine (4 mmol, 0.7 ml) (DIEA, Applied Biosystems, Product 400136) was added, and the suspension is agitated overnight. The resin was washed on a coarse glass scintered filter with dimethylformamide (20 ml×2) alternated with CH₂Cl₂ (20 ml×2) for 2 cycles. The above protocol was repeated to ensure quantitative coupling. The resin was dried under vacuum, and cleaved with 5 ml of a solution of triflouroacetic acid:H₂O:triopropylsilane 18:1:1 for 2-3 hr. The peptide-containing solution was filtered through a glass frit into a total volume of 100 ml of diethylether, and pelleted at 5000 rpm for 5 min. The pellet was washed with ether, and air dried. The pellet was then dissolved in dimethylsulfoxide, and the peptide was purified on a C18 reverse phase HPLC column developed with a 5-95% acetonitrile gradient in water. Fractions containing >95% pure target peptide substrate were pooled, and lyophilized to dryness. Overall yield was 30% (0.139 g, 0.075 mmol), 98% purity by analytical reversed phase HPLC. Electrospray mass spectrometry gave M+2H 924, corresponding to the expected exact mass of 1844.8992.

### Example 17

Preparation of N-2-[(7-methoxy-2-oxo-2H-chromen-4-yl)acetyl]-D-arginyl-L-prolyl-L-leucylglycyl-L-leucyl-3-[(2,4-dinitrophenyl)amino]-L-alanyl-beta-alanyl-D-arginyl-beta-alanyl-D-argininamide.

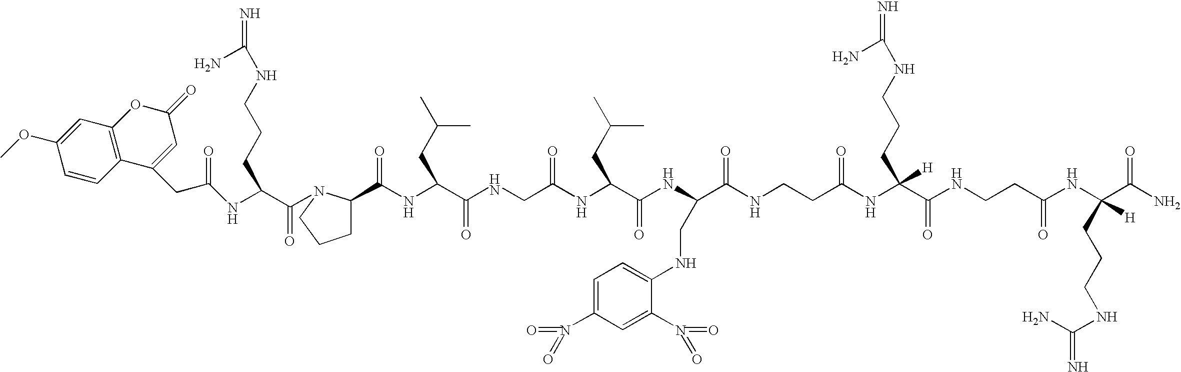

Part A. Preparation of beta-alanyl-D-arginyl-beta-alanyl-D-arginyl-resin.

Beta-alanyl-D-arginyl-beta-alanyl-D-arginyl-resin was synthesized attached to Applied Biosystems Fmoc-Amide-Resin (Product number 401435) using the Applied Biosystems model 433A Peptide Synthesizer and the manufacturer's reagents and reaction vessel designed for the 0.25 mM synthesis scale. Beta-alanine (Product 04-12-1044) and D-Arginine (Product 04-13-1045) were purchased from Novabiochem. The manufacturer's preprogrammed cycles were modified to increase each cycle reaction time by 30 min.

Part B. Preparation of 3-[(2,4-dinitrophenyl)amino]-L-alanyl-beta-alanyl-D-arginyl-beta-alanyl-D-arginyl-resin.

The product from Example 1, Part A (1 mmol, 0.492 g) is dissolved in 2 ml dimethylformamide. To this solution were added 1 mmol (0.135 g) 1-Hydroxy-7-azabenzo-triazole (HOAT, Aldrich, Product 44,545-2), 1 mmol (0.380 g) O-(7-Azabenzotriazol-1-yl)-N,N,N′,N′-tetramethyluronium hexafluorophosphate (HATU, Aldrich, Product 44,545-0), and 0.25 mmol of the product from Part A that had been swollen with 2.5 ml CH₂Cl₂ and diluted to 5 ml with dimethylformamide solvent. N,N,-Diopropylethylamine (4 mmol, 0.7 ml) (DIEA, Applied Biosystems, Product 400136) was added, the suspension was diluted to 10 ml final volume, and agitated overnight. The resin was washed on a coarse glass cintered filter with dimethylformamide (20 ml×2) alternated with CH₂Cl₂ (20ml×2) for 2 cycles. Unreacted amino groups remaining on the resin were then capped by dripping a solution through the resin on a coarse glass scintered filter containing 10 ml acetic anhydride (106 mmol), 10 ml N,N,-Diopropylethylamine (57 mmol) and 30 ml dimethylformamide. The resin was washed again as described, and returned to the automated synthesis reaction vessel for Fmoc removal and chain elongation.

Part C. Synthesis of D-arginyl-L-prolyl-L-leucylglycyl-L-leucyl-3-[(2,4-dinitrophenyl)amino]-L-alanyl-beta-alanyl-D-arginyl-beta-alanyl-D-arginyl-resin.

The product from Part B was elongated still attached to the resin using the Applied Biosystems Model 433A Synthesizer and the manufacturer's reagents and reaction vessel designed for the 0.25 mmol scale. The manufacturer's preprogrammed cycles were modified to increase each cycle reaction time by 30 min. L-leucine, glycine, L-leucine, L-proline, and D-arginine were added in order, and the amino-terminal Fmoc was removed prior to removal of the resin from the machine.

Part D. Preparation of N-2-[(7-methoxy-2-oxo-2H-chromen-4-yl)acetyl]-D-arginyl-L-prolyl-L-leucylglycyl-L-leucyl-3-[(2,4-dinitrophenyl)amino]-L-alanyl-beta-alanyl-D-arginyl-beta-alanyl-D-argininamide (C₆₄H₉₇N₂₃O₁₈).

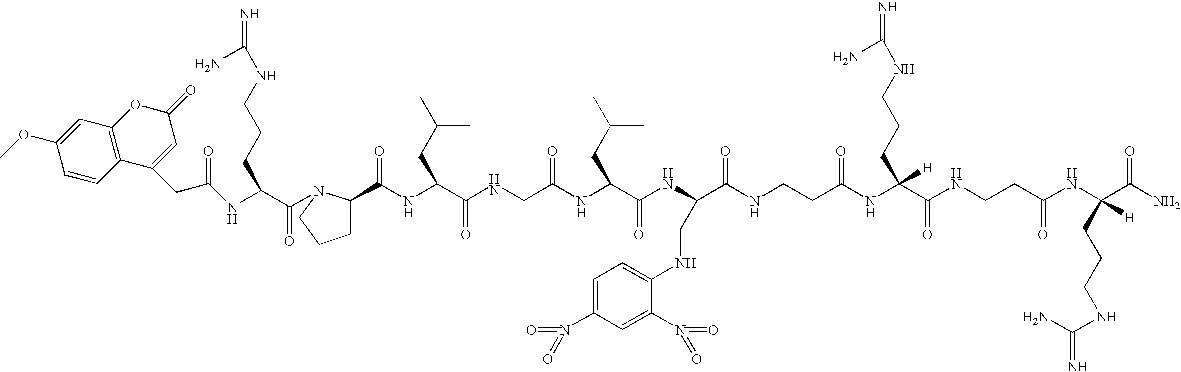

The 7-methoxycoumarin-4-acetic acid reagent (0.234 g, 1.0 mmol) (Aldrich, Product 23,519-9) was suspended in 5 ml dimethylformamide. To the suspension were added 1.0 mmol (0.136 g) 1-hydroxybenzotriazole hydrate (HOBT, Aldrich, Product 15726-0), 1.0 mmol (0.442 g) (benzotriazol-1-yloxy)tr(dimethylamino)phosphonium hexafluorophosphate (BOP, Castros Reagent, Perseptive Biosystems, Product GEN076503), and 0.25 mmol of product from Part C that had been swollen with CH₂Cl₂. N,N-Diopropylethylamine (2 mmol, 0.174 ml) (DIEA, Applied Biosystems, Product 400136) was added, and the suspension was agitated overnight. The resin was washed on a coarse glass scintered filter with dimethylformamide (20 ml×2) alternated with CH₂Cl₂ (20 ml×2) for 2 cycles. The resin was dried under vacuum, and cleaved with 5 ml of a solution of triflouroacetic acid:H₂O:triopropylsilane 18:1:1 for 2-3 hr. The peptide-containing solution was filtered through a glass frit into a total volume of 100 ml of diethyl ether, and pelleted at 5000 rpm for 5 min. The pellet was washed with ether, and air dried. The pellet was then dissolved in dimethylsulfoxide, and the peptide was purified on a C18 reverse phase HPLC column developed with a 5-95% acetonitrile gradient in water. Fractions containing >95% pure target peptide substrate were pooled, and lyophilized to dryness. Overall yield was 30% (0.139 g, 0.075 mmol), 98% purity by analytical reversed phase HPLC. Electrospray mass spectrometry gave M+H 1476.7 and M+2H 738.9, corresponding to the expected exact mass of 1476.61.

### Example 18

Preparation of N-2-[(7-methoxy-2-oxo-2H-chromen-4-yl)acetyl]-D-arginyl-L-prolyl-L-leucyl-S-methyl-L-cysteinyl-L-leucyl-3-[(2,4-dinitrophenyl)amino]-L-alanyl-L-alanyl-D-arginyl-D-alpha-glutamyl-D-argininamide.

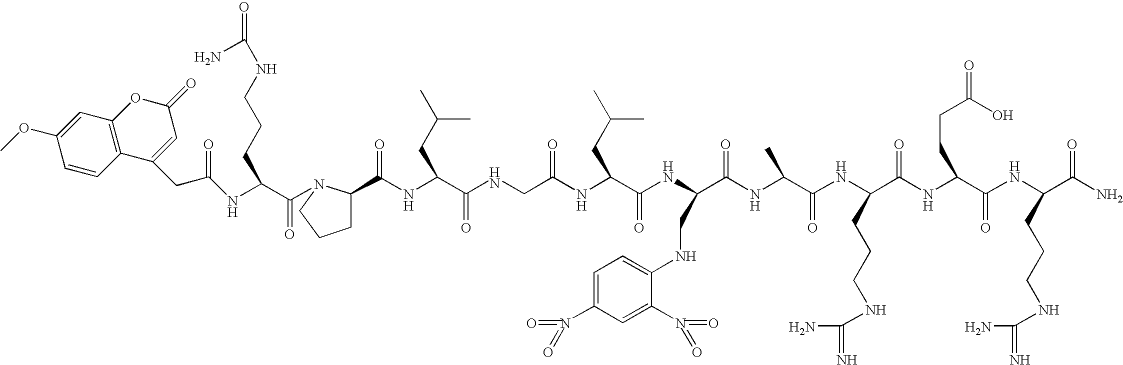

Part A. Preparation of D-arginyl-L-prolyl-L-leucyl-S-methyl-L-cysteinyl-L-leucyl-3-[(2,4-dinitrophenyl)amino]-L-alanyl-L-alanyl-D-arginyl-D-alpha-glutamyl-D-arginyl-resin.

The product from Example 11, Part B was elongated still attached to the resin using the Applied Biosystems Model 433A Synthesizer and the manufacturer's reagents and reaction vessel designed for the 0.25 mmol scale. The manufacturer's preprogrammed cycles were modified to increase each cycle reaction time by 30 min. L-leucine, S-methyl-L-cystine (Bachem, Product B-2510), L-leucine, L-proline, and D-arginine (Novabiochem, Product 04-13-1045) were added in order, and the amino-terminal Fmoc was removed prior to removal of the resin from the machine.

Part B. Preparation of N-2-[(7-methoxy-2-oxo-2H-chromen-4-yl)acetyl]-D-arginyl-L-prolyl-L-leucyl-S-methyl-L-cysteinyl-L-leucyl-3-[(2,4-dinitrophenyl)amino]-L-alanyl-L-alanyl-D-arginyl-D-alpha-glutamyl-D-argininamide (C₆₈H₁₀₃N₂₃O₂₀S).

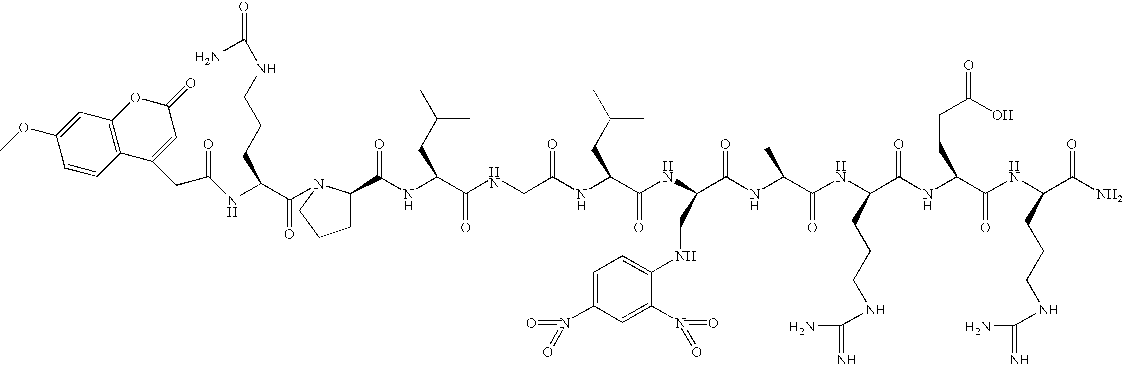

7-methoxycoumarin-4-acetic acid (MCA) was conjugated to the peptide on resin from Part A, and the peptide was cleaved from the resin, and purified as described in Example 1, Part E. Overall yield was 40% (0.158 g, 0.099 mmol), 93% purity by analytical reversed phase HPLC. Electrospray mass spectrometry gave M+2H 798.18, corresponding to the expected exact mass of 1593.7470.

### Example 19

Preparation of N-2-[(7-methoxy-2-oxo-2H-chromen-4-yl)acetyl]-N-(imidamidyl)-piperidin-3-yl-L-glycyl-L-prolyl-L-leucylglycyl-L-leucyl-3-[(2,4-dinitrophenyl)amino]-L-alanyl-L-alanyl-L-N-(imidamidyl)-piperidin-3-yl-L-glycyl-L-alpha-glutamyl-L-N-(imidamidyl)-piperidin-3-yl-glycylamide.

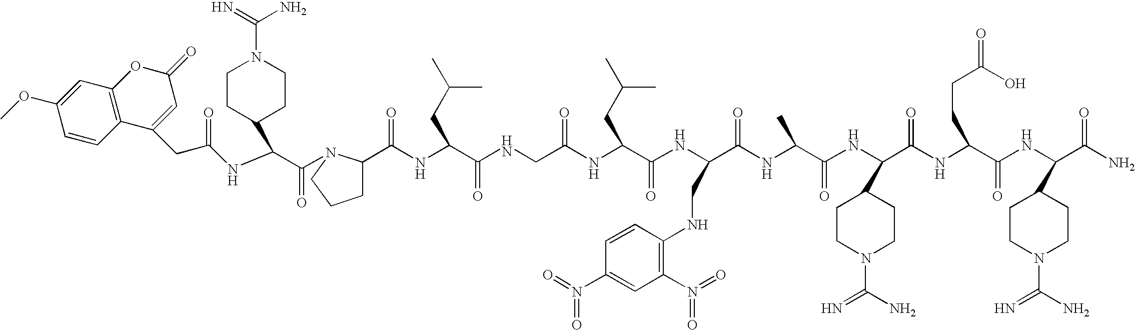

Part A. Preparation of N-(imidamidyl)-piperidin-3-yl-L-alanyl-L-prolyl-L-leucylglycyl-L-leucyl-3-[(2,4-dinitrophenyl)amino]-L-alanyl-L-alanyl-L-N-(imidamidyl)-piperidin-3-yl-L-alanyl-L-alpha-glutamyl-L-N-(imidamidyl)-piperidin-3-yl-alanyl-resin.

L-alanyl-L-N-(imidamidyl)-piperidin-3-yl-L-alanyl-L-alpha-glutamyl-L-N-(imidamidyl)-piperidin-3-yl-alanyl-resin was synthesized attached to Applied Biosystems Fmoc-Amide Resin (Product number 401435) using the Applied Biosystems Model 433A Synthesizer and the manufacturer's reagents and reaction vessel designed for the 0.25 mmol scale as described in Example 1, Part B. The manufacturer's preprogrammed cycles were modified to increase each cycle reaction time by 30 min. L-N-(imidamidyl)-piperidin-3-yl-alanine (RSP Amino Acid Analogues, Inc., Hopkinton, Mass., Product #6066-fp), L-glutamic acid, L-N-(imidamidyl)-piperidin-3-yl-alanine, and L-alanine were added to the resin in order, followed by conjugation of the product from Example 1, Part A, as described in Example 1, Part C. The resulting product was elongated still attached to the resin using the Applied Biosystems Model 433A Synthesizer and the manufacturer's reagents and reaction vessel designed for the 0.25 mmol scale. L-leucine, glycine, L-leucine, L-proline, and L-N-(imidamidyl)-piperidin-3-yl-alanine were added in order, and the amino-terminal Fmoc was removed prior to removal of the resin from the machine.

Part B. Preparation of N-2-[(7-methoxy-2-oxo-2H-chromen-4-yl)acetyl]-N-(imidamidyl)-piperidin-3-yl-L-glycyl-L-prolyl-L-leucylglycyl-L-leucyl-3-[(2,4-dinitrophenyl)amino]-L-alanyl-L-alanyl-L-N-(imidamidyl)-piperidin-3-yl-L-glycyl-L-alpha-glutamyl-L-N-(imidamidyl)-piperidin-3-yl-glycylamide (C₇₂H₁₀₅N₂₃O₂₀).

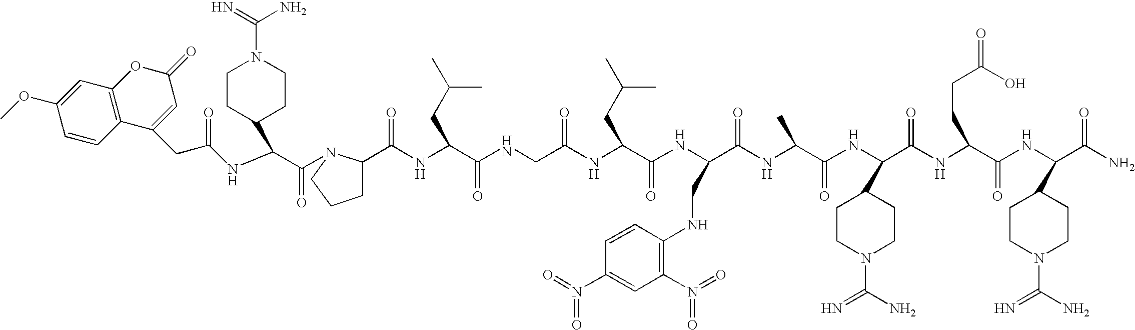

7-methoxycoumarin-4-acetic acid (MCA) was conjugated to the peptide on resin from Part A, and the peptide was cleaved from the resin and purified as described in Example 1, Part E. Overall yield was 15% (0.060 g, 0.037 mmol), 96% purity by analytical reversed phase HPLC. Electrospray mass spectrometry gave M+2H 806.5, corresponding to the expected exact mass of 1611.

### Example 20

Preparation of N-2-[(7-methoxy-2-oxo-2H-chromen-4-yl)acetyl]-L-arginyl-L-prolyl-L-leucylglycyl-6-(benzyloxy)-L-norleucyl-3-[(2,4-dinitrophenyl)amino]-L-alanyl-L-alanyl-L-arginyl-L-alpha-glutamyl-L-argininamide.

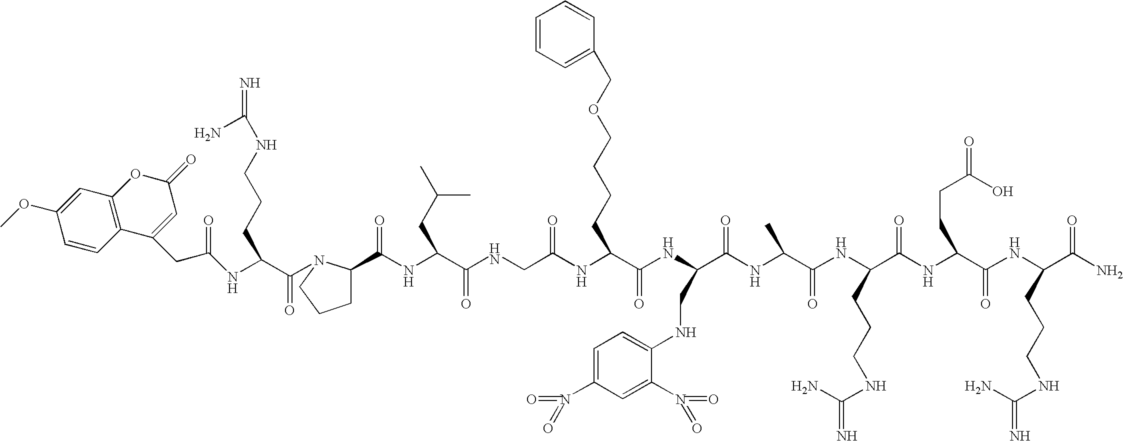

Part A. Preparation of L-arginyl-L-prolyl-L-leucylglycyl-6-(benzyloxy)-L-norleucyl-3-[(2,4-dinitrophenyl)amino]-L-alanyl-L-alanyl-L-arginyl-L-alpha-glutamyl-L-arginyl-resin.

The 6-(benzyloxy)-N-[(9H-fluoren-9-ylmethoxy)carbonyl]-L-norleucine from Example 8, Part B was conjugated to the resin from Example 1, Part C as described in Example 8, Part C. The resin-bound product was elongated still attached to the resin using the Applied Biosystems Model 433A Synthesizer and the manufacturer's reagents and reaction vessel designed for the 0.25 mmol scale. The manufacturer's preprogrammed cycles were modified to increase each cycle reaction time by 30 min. Glycine, L-leucine, L-proline, and L-arginine (Novabiochem, Product 04-13-1045) were added in order, and the amino-terminal Fmoc was removed prior to removal of the resin from the machine.

Part B. Preparation of N-2-[(7-methoxy-2-oxo-2H-chromen-4-yl)acetyl]-L-arginyl-L-prolyl-L-leucylglycyl-6-(benzyloxy)-L-norleucyl-3-[(2,4-dinitrophenyl)amino]-L-alanyl-L-alanyl-L-arginyl-L-alpha-glutamyl-L-argininamide (C₇₃H₁₀₅N₂₃O₂₁).

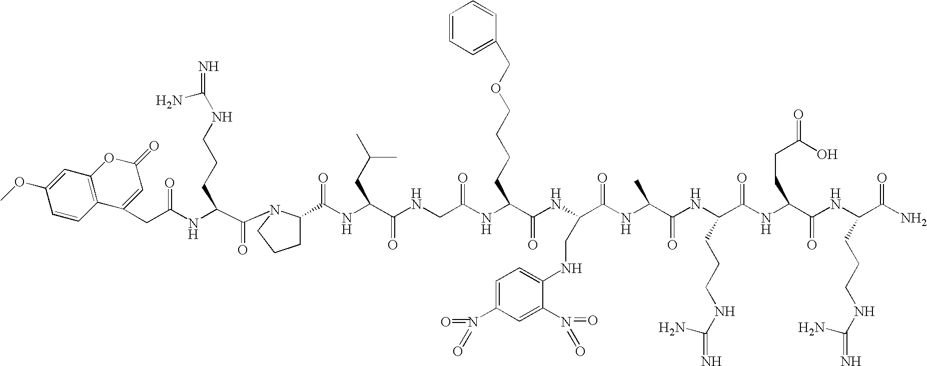

7-methoxycoumarin-4-acetic acid (MCA) was conjugated to the product from Part A, and the peptide was cleaved from the resin and purified as described in Example 1, Part E. Overall yield was 39% (0.160 g, 0.098 mmol), 98% purity by analytical reversed phase HPLC. Electrospray mass spectrometry gave M+2H 821.1, corresponding to the expected exact mass of 1639.7855.

### Example 21

Preparation of N-2-[(7-methoxy-2-oxo-2H-chromen-4-yl)acetyl]-D-arginyl-L-prolyl-L-leucylglycyl-6-(benzyloxy)-L-norleucyl-3-[(2,4-dinitrophenyl)amino]-L-alanyl-L-alanyl-D-arginyl-D-alpha-glutamyl-D-argininamide (C₇₃H₁₀₅N₂₃O₂₁).

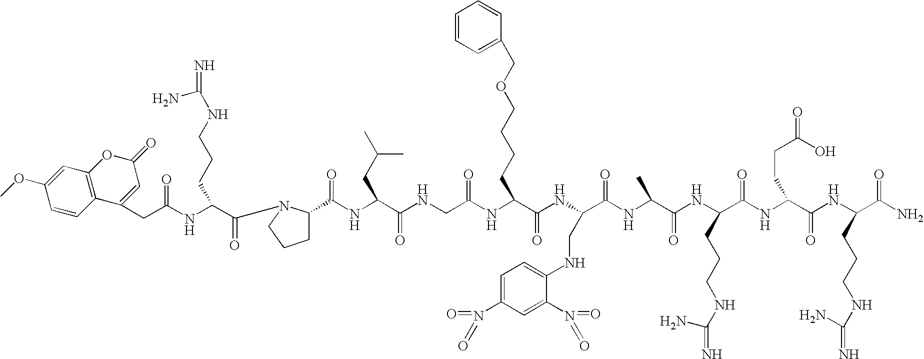

N-2-[(7-methoxy-2-oxo-2H-chromen-4-yl)acetyl]-D-arginyl-L-prolyl-L-leucylglycyl-6-(benzyloxy)-L-norleucyl-3-[(2,4-dinitrophenyl)amino]-L-alanyl-L-alanyl-D-arginyl-D-alpha-glutamyl-D-argininamide was prepared as described in Example 20, except that D-arginine was substituted for L-arginine, and D-glutamic acid was substituted for L-glutamic acid. Overall yield was 37% (0.150 g, 0.092 mmol), 98% purity by analytical reversed phase HPLC. Electrospray mass spectrometry gave M+2H 821.3, corresponding to the expected exact mass of 1639.7855.

### Example 22

Preparation of N-2-[(7-methoxy-2-oxo-2H-chromen-4-yl)acetyl]-D-arginyl-L-prolyl-L-glutaminyl-L-alpha-glutamyl-6-(benzyloxy)-L-norleucyl-3-[(2,4-dinitrophenyl)amino]-L-alanyl-L-alanyl-D-arginyl-D-alpha-glutamyl-D-argininamide (C₇₅H₁₀₆N₂₄O₂₄).

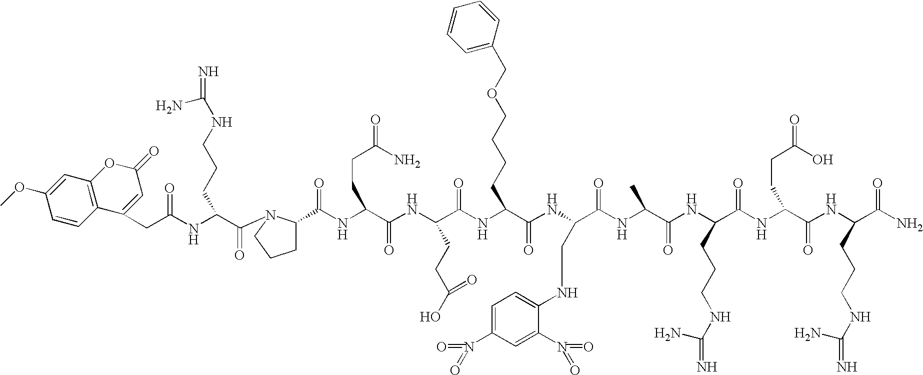

N-2-[(7-methoxy-2-oxo-2H-chromen-4-yl)acetyl]-D-arginyl-L-prolyl-L-glutaminyl-L-alpha-glutamyl-6-(benzyloxy)-L-norleucyl-3-[(2,4-dinitrophenyl)amino]-L-alanyl-L-alanyl-D-arginyl-D-alpha-glutamyl-D-argininamide was prepared as described in Example 21, except that L-leucine was replaced by L-glutamine, and glycine was replaced by L-glutamic acid. Overall yield was 28% (0.121 g, 0.070 mmol), 98% purity by analytical reversed phase HPLC. Electrospray mass spectrometry gave M+2H 864.4, corresponding to the expected exact mass of 1726.7812.

### Example 23

Preparation of N-2-[(7-methoxy-2-oxo-2H-chromen-4-yl)acetyl]-D-arginyl-L-prolyl-L-leueyl-L-alpha-glutamyl-6-(benzyloxy)-L-norleueyl-3-[(2,4-dinitrophenyl)amino]-L-alanyl-L-alanyl-D-arginyl-D-alpha-glutamyl-D-argininamide.

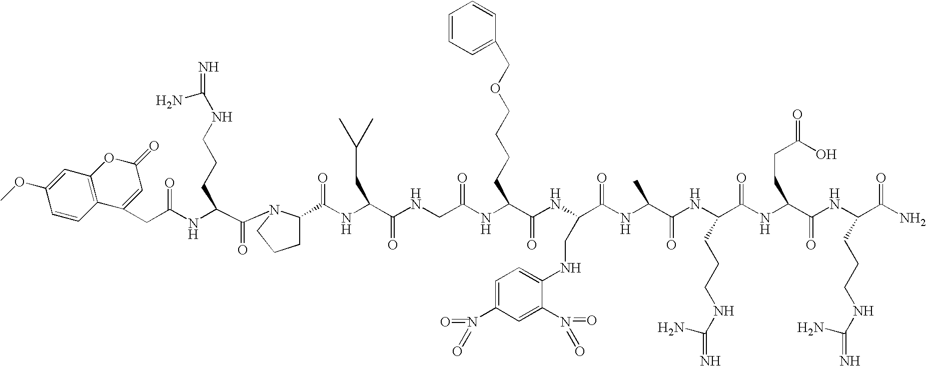

N-2-[(7-methoxy-2-oxo-2H-chromen-4-yl)acetyl]-D-arginyl-L-prolyl-L-leucyl-L-alpha-glutamyl-6-(benzyloxy)-L-norleucyl-3-[(2,4-dinitrophenyl)amino]-L-alanyl-L-alanyl-D-arginyl-D-alpha-glutamyl-D-argininamide was prepared as described in Example 21, except that glycine was replaced by L-glutamic acid. Overall yield was 22% (0.096 g, 0.056 mMoles), 98% purity by analytical reversed phase HPLC. Electrospray mass spectrometry gave M+2H 856.9, corresponding to the expected exact mass of 1711.8067.

### Example 24

Preparation of N-2-[(7-methoxy-2-oxo-2H-chromen-4-yl)acetyl]-D-arginyl-L-prolyl-L-glutaminylglycyl-6-(benzyloxy)-L-norleucyl-3-[(2,4-dinitrophenyl)amino]-L-alanyl-L-alanyl-D-arginyl-D-alpha-glutamyl-D-argininamide (C₇₂H₁₀₂N₂₄O₂₂).

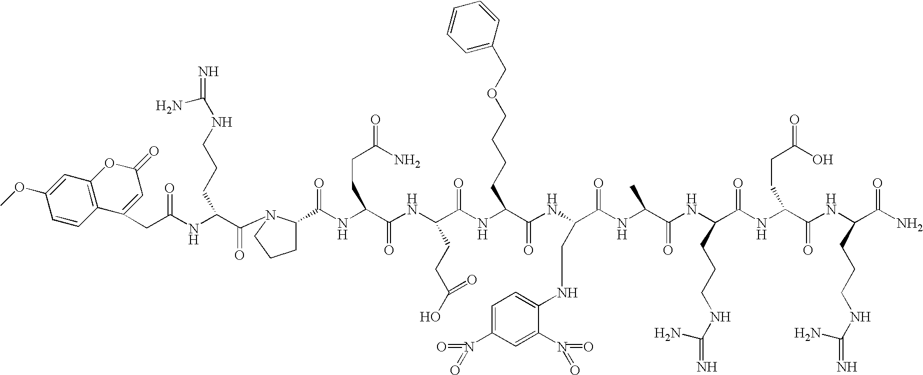

N-2-[(7-methoxy-2-oxo-2H-chromen-4-yl)acetyl]-D-arginyl-L-prolyl-L-glutaminylglycyl-6-(benzyloxy)-L-norleucyl-3-[(2,4-dinitrophenyl)amino]-L-alanyl-L-alanyl-D-arginyl-D-alpha-glutamyl-D-argininamide was prepared as described in Example 21, except that L-leucine was replaced by L-glutamine. Overall yield was 31% (0.128 g, 0.077 mmol), 98% purity by analytical reversed phase HPLC. Electrospray mass spectrometry gave M+2H 828.4, corresponding to the expected exact mass of 1654.7601.

### Example 25

Preparation of N-2-[(7-methoxy-2-oxo-2H-chromen-4-yl)acetyl]-D-arginyl-L-prolyl-L-leucylglycyl-6-phenoxy-L-norleucyl-3-[(2,4-dinitrophenyl)amino]-L-alanyl-L-alanyl-D-arginyl-D-alpha-glutamyl-D-argininamide.

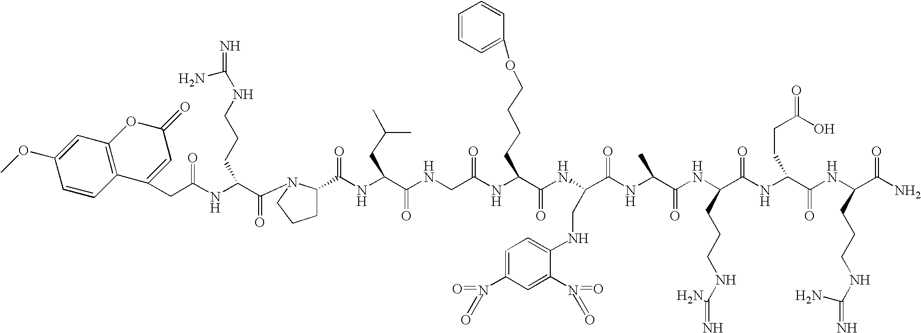

Part A. Preparation of benzyl-N-[(9H-fluoren-9-ylmethoxy)carbonyl]-6-phenoxynorleucinate.

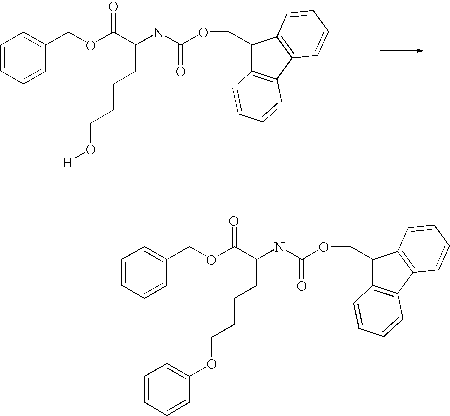

To a solution of the product from Example 8, Part B (1.0 g, 2.2 mmol) and phenol (300 mg, 3.2 mmol) in toluene (5 ml) were added diethyl azodicarboxylate (DEAD) (0.4 g, 2.3 mmol) and triphenylphosphine (0.6 g, 2.3 mmol). The resulting mixture was left to stir at ambient temperature for 12-15 hr (the course of the reaction was monitored by RPHPLC). After the completion of the reaction, the solution was concentrated via rotovaparator, then purified on SiO2 to result in a clear oil (2 g, 98%). ¹H NMR and Mass spectroscopy were consistent with the desired structure. Mass spectroscopy showed: C₃₄H₃₃NO₅−M^{+Na+H}_{found}=558.20(M^{+H}_{calc}=558).

Part B. Preparation of 1-N-[(9H-fluoren-9-ylmethoxy)carbonyl]-6-phenoxynorleucine.

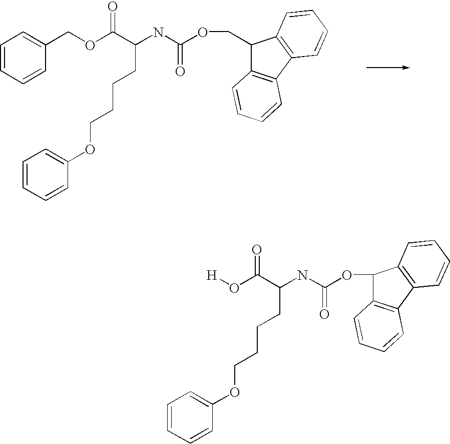

To a methanol (50 ml) solution of the product from Part A (1.2 g) was added 10% Pd on carbon (600 mg, Degussa type catalyst). The black mixture was shaken on a Parr apparatus for 2.5 hr @50 psi. After the completion of the reaction, the mixture was filtered through a celite pad. The solvent was removed under reduced pressure to give 800 mg of the N-FMOC amino acid. ¹H NMR and Mass spectroscopy were consistent with the desired structure. Mass spectroscopy showed: C₂₃H₂₇NO₅−M^{+Na+H}_{found}=556.20 (M^{+Na+H}_{calc}=556).

Part C. Preparation of N-2-[(7-methoxy-2-oxo-2H-chromen-4-yl)acetyl]-D-arginyl-L-prolyl-L-leucylglycyl-6-phenoxy-L-norleucyl-3-[(2,4-dinitrophenyl)amino]-L-alanyl-L-alanyl-D-arginyl-D-alpha-glutamyl-D-argininamide (C₇₂H₁₀₃N₂₃O₂₁).

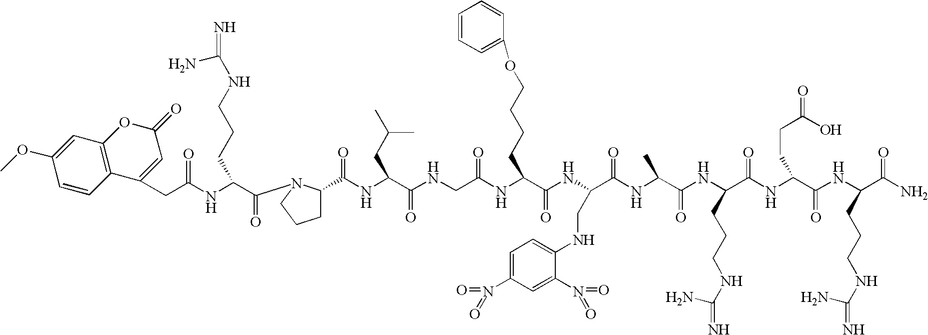

The product from Part B above was manually conjugated to a 1.0 mmol preparation of the product from Example 11, Part B, following the manual coupling protocol described in Example 8, Part C. The substrate was further elongated, and purified as described for Example 21 Overall yield was 41% (0.0.662 g, 0.407 mmol), 96% purity by analytical reversed phase HPLC. Electrospray mass spectrometry gave M+2H 813.8, M+H+Na 824.8, M+3H 543.1, corresponding to the expected exact mass of 1625.7699.

### Example 26

Preparation of N-2-[(7-methoxy-2-oxo-2H-chromen-4-yl)acetyl]-D-arginyl-L-prolyl-L-leucylglycyl-S-(4-methoxybenzyl)-L-cysteinyl-3-[(2,4-dinitrophenyl)amino]-L-alanyl-L-alanyl-D-arginyl-D-alpha-glutamyl-D-argininamide (C₇₁H₁₀₁N₂₃O₂₁S).

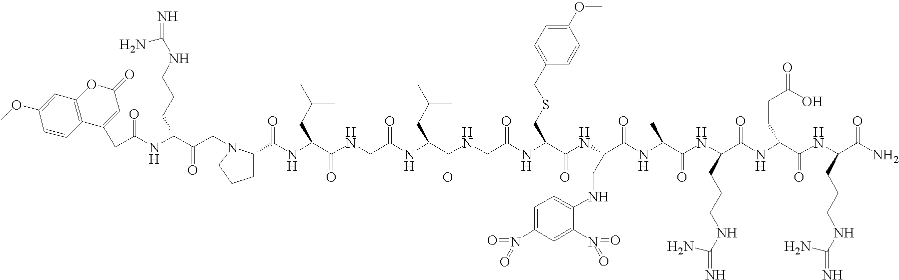

N-2-[(7-methoxy-2-oxo-2H-chromen-4-yl)acetyl]-D-arginyl-L-prolyl-L-leucylglycyl-S-(4-methoxybenzyl)-L-cysteinyl-3-[(2,4-dinitrophenyl)amino]-L-alanyl-L-alanyl-D-arginyl-D-alpha-glutamyl-D-argininamide was prepared as described in Example 21, except that S-(4-methoxybenzyl)-L-cystine was substituted for 6-(benzyloxy)-L-norleucine. Overall yield was 29% (0.120 g, 0.092 mmol), 98% purity by analytical reversed phase HPLC. Electrospray mass spectrometry gave M+2H 823.6, corresponding to the expected exact mass of 1644.7263.

### Example 27

Preparation of N-2-[(7-methoxy-2-oxo-2H-chromen-4-yl)acetyl]-N-(imidamidyl)-piperidin-3-yl-L-glycyl-L-prolyl-L-leucylglycyl-6-(benzyloxy)-L-norleucyl-3-[(2,4-dinitrophenyl)amino]-L-alanyl-L-alanyl-L-N-(imidamidyl)-piperidin-3-yl-L-glycyl-L-alpha-glutamyl-L-N-(imidamidyl)-piperidin-3-yl-glycylamide.

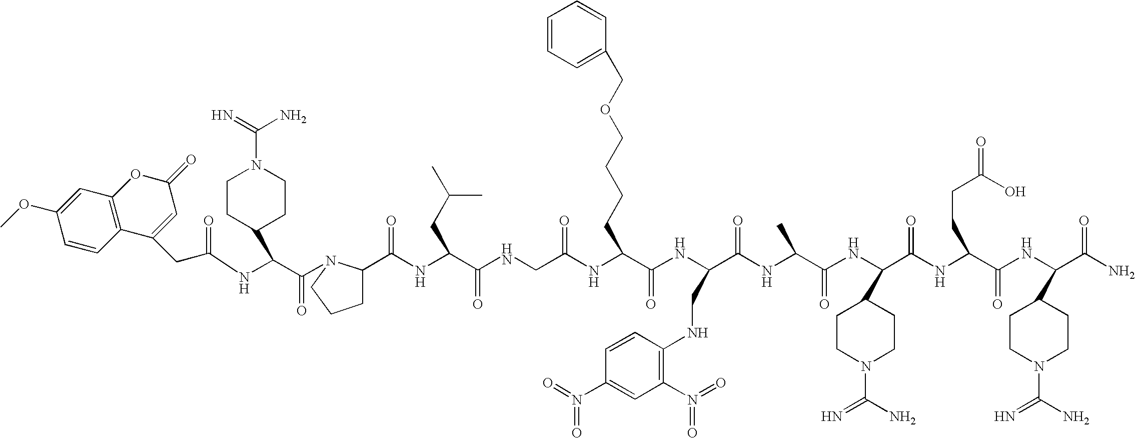

Part A. Preparation of N-(imidamidyl)-piperidin-3-yl-L-alanyl-L-prolyl-L-leucylglycyl-6-(benzyloxy)-L-norleucyl-3-[(2,4-dinitrophenyl)amino]-L-alanyl-L-alanyl-L-N-(imidamidyl)-piperidin-3-yl-L-alanyl-L-alpha-glutamyl-L-N-(imidamidyl)-piperidin-3-yl-alanyl-resin.

L-alanyl-L-N-(imidamidyl)-piperidin-3-yl-L-alanyl-L-alpha-glutamyl-L-N-(imidamidyl)-piperidin-3-yl-alanyl-resin was synthesized attached to Applied Biosystems Fmoc-Amide Resin (Product number 401435) using the Applied Biosystems Model 433A Synthesizer and the manufacturer's reagents and reaction vessel designed for the 0.25 mmol scale as described in Example 1, Part B. The manufacturer's preprogrammed cycles were modified to increase each cycle reaction time by 30 min. L-N-(imidamidyl)-piperidin-3-yl-alanine (RSP Amino Acid Analogues, Inc., Hopkinton, Mass., Product #6066-fp), L-glutamic acid, L-N-(imidamidyl)-piperidin-3-yl-alanine, and L-alanine were added to the resin in order, followed by conjugation of the product from Example 1, Part A, as described in Example 1, Part C. The resulting product was elongated still attached to the resin using the Applied Biosystems Model 433A Synthesizer and the manufacturer's reagents and reaction vessel designed for the 0.25 mmol scale. 6-(benzyloxy)-L-norleucine, glycine, L-leucine, L-proline, and L-N-(imidamidyl)-piperidin-3-yl-alanine were added in order, and the amino-terminal Fmoc was removed prior to removal of the resin from the machine.

Part B. Preparation of N-2-[(7-methoxy-2-oxo-2H-chromen-4-yl)acetyl]-N-(imidamidyl)-piperidin-3-yl-L-glycyl-L-prolyl-L-leucylglycyl-6-(benzyloxy)-L-norleucyl-3-[(2,4-dinitrophenyl)amino]-L-alanyl-L-alanyl-L-N-(imidamidyl)-piperidin-3-yl-L-glycyl-L-alpha-glutamyl-L-N-(imidamidyl)-piperidin-3-yl-glycylamide (C₇₉H₁₁₁N₂₃O₂₁).

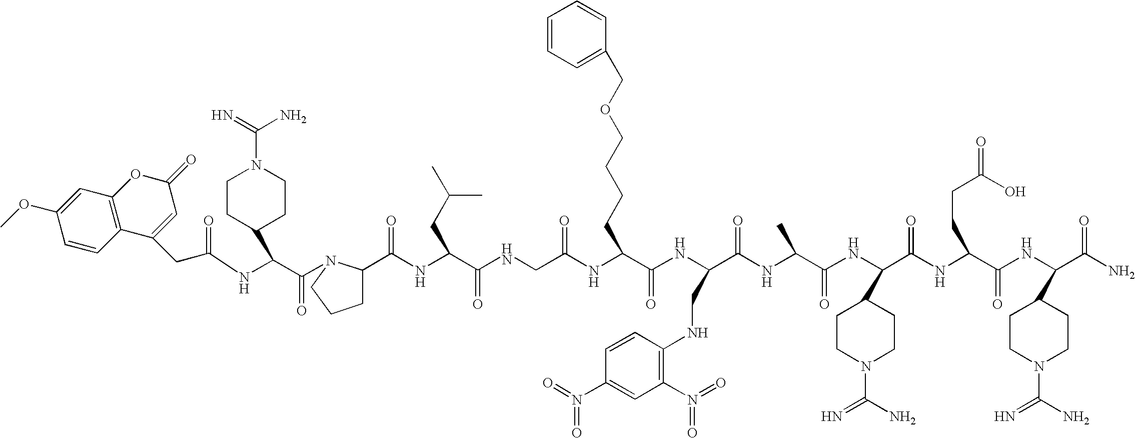

7-methoxycoumarin-4-acetic acid (MCA) was conjugated to the peptide from Part A, and the peptide was cleaved from the resin, and purified as described in Example 1, Part E. Overall yield was 2% (0.009 g, 0.005 mmol), 94% purity by analytical reversed phase HPLC. Electrospray mass spectrometry gave M+2H 859.89, corresponding to the expected exact mass of 1717.8325.

### Example 28

Preparation of N-2-[5-(2-oxohexahydro-1H-thieno[3,4-d]imidazol-4-yl)pentanoyl]-L-arginyl-L-prolyl-L-leucylglycyl-L-leucyl-N-6-(4-{[3-carboxy-4-(6-hydroxy-3-oxo-3H-xanthen-9-yl)phenyl]amino}-6-hydroxy-1,3,5-triazin-2-yl)-L-lysyl-L-alanyl-L-arginyl-L-alpha-glutamyl-L-argininamide (C₈₆H₁₂₅N₂₇O₂₀S).

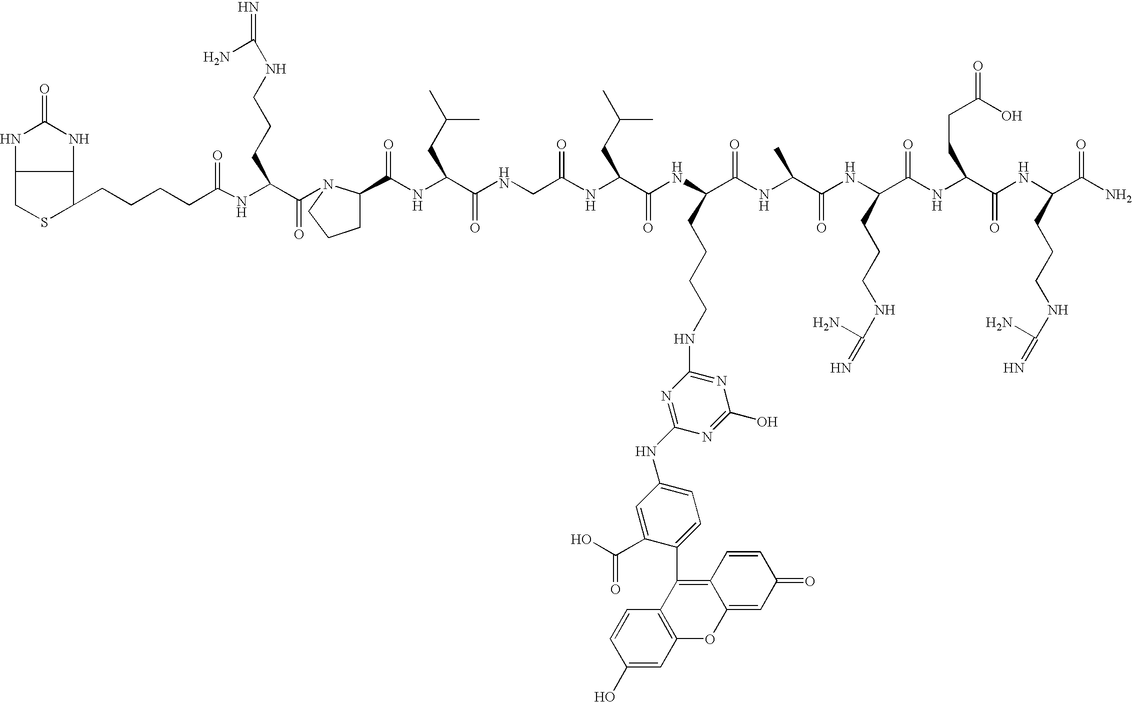

An aliquot (0.25 ml) of 1 molar potassium tert-butoxide in butanol was added to 3.5 ml of the crude reaction solution from Example 3, and the reaction was refluxed at 50° C. for 30 min. The solution turned vivid orange as the hydrolysis proceeded. The product was precipitated in 50 ml ether ,and pelleted at 5000 rpm for 5 min. The pellet was dissolved in dimethylsulfoxide, and the peptide was purified on a C18 reverse phase column developed with a 5-95% acetonitrile gradient in water. Fractions containing >95% pure target peptide substrate were lyophilized to dryness. The hydrolysis was only 10% complete. Yield was 9% (0.002 g, 0.001 mmol). Electrospray mass spectrometry gave M+2H 931.0 and M+3H 621.3 corresponding to the expected exact mass of 1859.9002. Fluorescence excitation scans were performed at fixed emission 515 nM, and emission scans were performed at fixed excitation 495 nM. Optimum was found to be unchanged from that of Example 2.

### Example 29

Preparation of N-2-[(7-methoxy-2-oxo-2H-chromen-4-yl)acetyl]-L-arginyl-L-prolyl-L-glutaminyl-L-alpha-glutamyl-6-(benzyloxy)-L-norleucyl-3-[(2,4-dinitrophenyl)amino]-L-alanyl-L-alanyl-L-arginyl-L-alpha-glutamyl-L-argininamide (C₇₅H₁₀₆N₂₄O₂₄).

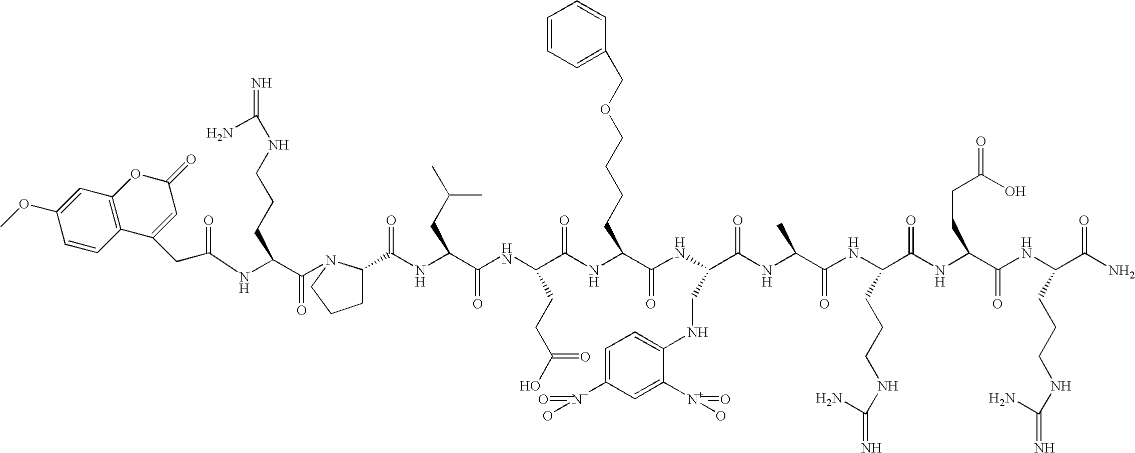

N-2-[(7-methoxy-2-oxo-2H-chromen-4-yl)acetyl]-L-arginyl-L-prolyl-L-glutaminyl-L-alpha-glutamyl-6-(benzyloxy)-L-norleucyl-3-[(2,4-dinitrophenyl)amino]-L-alanyl-L-alanyl-L-arginyl-L-alpha-glutamyl-L-argininamide was prepared as described in Example 22, except that L-arginine was substituted for D-arginine, and L-glutamic acid was substituted for D-glutamic acid. Overall yield was 32% (0.140 g, 0.081 mmol), 99.9% purity by analytical reversed phase HPLC. Electrospray mass spectrometry gave M+2H 864.41, M+3H 576.95, corresponding to the expected exact mass of 1726.7812.

### Example 30

In Vitro MMP Inhibition Analysis.

Several hydroxamate compounds were analyzed in in vitro assays to determine their ability to inhibit the MMP cleavage of peptide substrates. Inhibition constants (Kᵢ) were calculated from the assayed hydroxamate-MMP interactions.

Human recombinant MMP-1, MMP-2, MMP-9, MMP-13, and MMP-14 were used in this assay. The enzymes are prepared following known laboratory procedures. Protocols for the preparation and use of these enzymes are available in the scientific literature. See, e.g., Enzyme Nomenclature (Academic Press, San Diego, Calif., 1992) (and the citations therein). See also, Frije et al., J. Biol. Chem., 26(24), 16766-73 (1994).

In addition, many MMPs may be purchased from suppliers. For example, MMP-1, MMP-2, MMP-3, MMP-7, MMP-8, MMP-9, MMP-10, MMP-12, and MMP-13 are commercially available from R&D Systems in their 2003 catalog.

The MMP-1 proenzyme may be purified from spent media of MMP-1-transfected HT-1080 cells and the protein purified on a zinc chelating column. The MMP-2 proenzyme may be purified by gelatin Sepharose chromatography from MMP-2-transfected p2AHT2 cells. The MMP-9 proenzyme may be purified by gelatin Sepharose chromatography from spent media of MMP-9-transfected HT1080 cells.

The MMP-13 may be obtained as a proenzyme from a full-length cDNA clone using baculovirus, as described by V. A. Luckow, “Insect Cell Expression Technology,” Protein Engineering: Principles and Practice, pp. 183-218 (edited by J. L. Cleland et al., Wiley-Liss, Inc., 1996). The expressed proenzyme was first purified over a heparin agarose column, and then over a chelating zinc chloride column. Further details on baculovirus expression systems may be found in, for example, Luckow et al., J. Virol., 67, 4566-79 (1993). See also, O'Reilly et al, Baculovirus Expression Vectors: A Laboratory Manual (W. H. Freeman and Co., New York, N.Y., 1992). See also, King et al., The Baculovirus Expression System: A Laboratory Guide (Chapman & Hall, London, England, 1992).

The full length MMP-14 cDNA may be used to express the catalytic domain enzyme in E. coli inclusion bodies. Then the enzyme is solubilized in urea, purified on a preparative C-14 reverse phase HPLC column, and refolded in the presence of zinc acetate and purified for use.

All MMPs were activated using 4-aminophenylmercuric acetate (“APMA”, Sigma Chemical, St. Louis, Mo.) or trypsin. MMP-9 also was activated using human recombinant MMP-3 following standard cloning and purification techniques.

The fluorogenic, methoxycoumarin-containing polypeptide substrate MCA-ArgProLeuGlyLeuDpaAlaArgGluArgNH₂ (compound 1 of Table 4) was used as the MMP substrate in the MMP inhibition assay. Here, “MCA” is 7-methoxycoumarin-4-yl acetyl and “Dpa” is 3-(2,4-dinitrophenyl)-L-2,3-diaminopropionyl group. In the absence of MMP inhibitory activity, the substrate is cleaved at the Gly-Leu peptide bond. The cleavage separates the highly fluorogenic peptide from the 2,4-dinitrophenyl quencher, resulting in increase of fluorescent intensity.

Stock solutions of the hydroxamate inhibitors (or salts thereof) were prepared in 1% dimethyl sulfoxide (DMSO). The stock solutions were diluted in Buffer A (100 mM Tris-HCl, 100 mM NaCl, 10 mM CaCl₂, 0.05% polyoxyethylene 23 lauryl ether, pH 7.5) to obtain solutions with different hydroxamate concentrations, i.e., assay solutions with different concentrations of the assayed MMP inhibitory compound. The experiment controls contained the same amount of Buffer A/DMSO as the assayed sample, but contained no hydroxamate inhibitor.

To determine Kᵢ, the inhibitor samples are incubated at room temperature for 1 hr in the presence of 4 μM of MMP substrate, and analyzed on a Tecan SpectraFlour Plus plate reader. The excitation wavelength is 330 nm, and the emission (fluorescence) wavelength is 420 nm. In the absence of MMP inhibitory activity, the substrate is cleaved at the Gly-Leu bond resulting in an increase of relative fluorescence. Inhibition is observed as a reduced rate of increase in relative fluorescence.

The inhibitors are analyzed using a single low enzyme concentration with a single substrate concentration fixed at or below the Kₘ. This protocol is a modification of method by Knight et al., FEBS Lett., 296(3), 263-266 (1992). Apparent inhibitory constants are determined by non-linear regression of reaction velocity as a function of inhibitor and enzyme concentration using Morrison's equation, as described by Kuzmic, Anal. Biochem. 286, 45-50 (2000). Modifications were made in the non-linear regression method to allow a common control reaction rate and effective enzyme concentration to be shared between all dose-response relationships on a given assay plate. Since the substrate concentration was chosen to be at or below the Kₘ, the apparent Kᵢ's from this analysis were reported as Kᵢ's without correction for the influence of substrate.

Table 4 below shows the Kᵢ values of several inhibitors using the above assay with MMP-1 MMP-2, MMP-9, MMP-13, and MMP-14. All values in Table 4 are given in nM units.

TABLE 4
MMP Inhibition Assay Results

| | | MMP-1 | MMP-2 | MMP-9 | MMP-13 | MMP-14 |
|---|---|---|---|---|---|---|
| No. | Structure | Kᵢ (nM) | Kᵢ (nM) | Kᵢ (nM) | Kᵢ (nM) | Kᵢ (nM) |
| 1 | 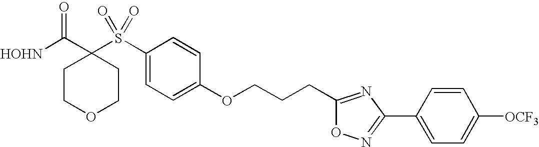 | >10000 | 412.93 | 1596.8 | 1.503 | >10000 |
| 2 | 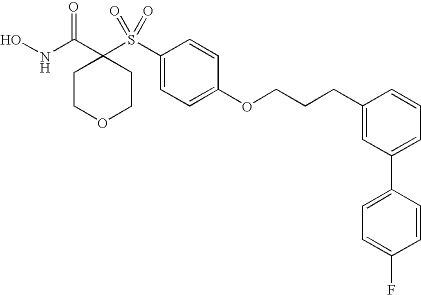 | >10000 | 1640 | 2360 | 3.04 | >10000 |
| 3 | 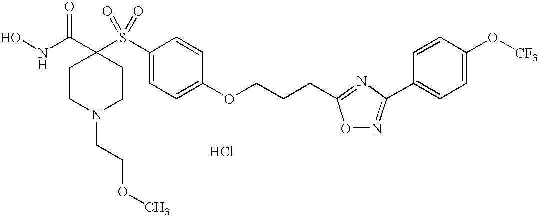 | >10000 | 186.28 | 661.7 | 0.486 | >10000 |

The above detailed description of preferred embodiments is intended only to acquaint others skilled in the art with the invention, its principles, and its practical application so that others skilled in the art may adapt and apply the invention in its numerous forms, as they may be best suited to the requirements of a particular use. This invention, therefore, is not limited to the above embodiments, and may be variously modified.

With reference to the use of the words “comprise” or “comprises” or “comprising” in this patent (including the claims), Applicants note that unless the context requires otherwise, those words are used on the basis and clear understanding that they are to be interpreted inclusively, rather than exclusively, and that Applicants intend each of those words to be so interpreted in construing this patent, including the claims below.

All references cited in this specification are hereby incorporated by reference. The discussion of the references herein is intended merely to summarize the assertions made by their authors, and no admission is made that any reference constitutes prior art.

We claim:

1. A compound or a salt thereof, wherein:
the compound comprises a peptide and corresponds in structure to Formula (I):
aa₍ᵢ₎-X—Y-aa₍ⱼ₎-Z (I);
aa₍ᵢ₎ comprises a sequence of i amino acids at the N-terminus of the peptide;
aa₍ⱼ₎ comprises a sequence of j amino acids at the C-terminus of the peptide;
i is an integer from 0 to 5;
j is an integer from 1 to 6;
a fluorophore is covalently attached to the peptide on one side of the X—Y bond, and at least one of a fluorescence quencher and a ligand is covalently attached to the peptide on the other side of the X—Y bond;
X comprises an MMP recognition sequence;
X is not Pro-Gln-Gln, Pro-Tyr-Ala, or Pro-Val-Glu;
Y comprises an amino acid; and
Z is:
a hydroxyl group at the C-terminus of the peptide, or
a protecting group at the C-terminus of the peptide.

2. A compound or salt thereof according to claim 1, wherein Y comprises a side chain comprising at least 8 non-hydrogen atoms.

3. A compound or salt thereof according to claim 2, wherein Y comprises a side chain comprising at least 10 non-hydrogen atoms.

4. A compound or salt thereof according to claim 3, wherein Y comprises a side chain comprising at least 11 non-hydrogen atoms.

5. A compound or salt thereof according to claim 4, wherein Y comprises a side chain comprising from 11 to 15 non-hydrogen atoms.

6. A compound or salt thereof according to claim 4, wherein Y comprises a side chain comprising at least 12 non-hydrogen atoms.

7. A compound or salt thereof according to claim 1, wherein the compound or salt is characterized in that MMP-2, MMP-9, or MMP-13 reacts with the compound or salt to cleave the compound or salt at the bond between X and Y when the compound or salt is combined with the MMP-2, MMP-9, or MMP-13.

8. A compound or salt thereof according to claim 7, wherein:
k_{cat}/Kₘ of the compound or salt with at least one of MMP-1 and MMP-7 is no greater than about 0.5×10⁻⁴M⁻¹s⁻¹, and
k_{cat}/Kₘ of the compound or salt with at least one of MMP-2, MMP-9, and MMP-13 is at least about 5×10⁻⁴M⁻¹s⁻¹.

9. A compound or salt thereof according to claim 8, wherein:
k_{cat}/Kₘ of the compound or salt with at least one of MMP-1 and MMP-7 is no greater than about 10⁻⁵ M⁻¹s⁻¹, and
k_{cat}/Kₘ of the compound or salt with at least one of MMP-2, MMP-9, and MMP-13 is at least about 50×10⁻⁴M⁻¹s⁻¹.

10. A compound or salt thereof according to claim 7, wherein the compound or salt is characterized in that the rate of reaction of the compound or salt with MMP-1 or MMP-7 is about zero.

11. A compound or salt thereof according to claim 10, wherein the compound or salt is characterized in that the rate of reaction of the compound or salt with MMP-1 and MMP-7 is about zero.

12. A compound or salt thereof according to claim 7, wherein Y is selected from the group consisting of 3-(2-napthyl)-L-alanine, O-benzyl-L-tyrosine, 6-(benzyloxy)-L-norleucine, S-(3-phenylpropyl)-L-cysteine, 6-phenoxy-L-norleucine and S-(4-methoxybenzyl)-L-cysteine.

13. A compound or salt thereof according to claim 7, wherein Y is selected from the group consisting of 6-(benzyloxy)-L-norleucine and 6-phenoxy-L-norleucine.

14. A compound or salt thereof according to claim 7, wherein the recognition sequence of X is selected from the group consisting of Ala-Gln-Gly, Ala-Met-His, Asn-Gln-Gly, Asp-Lys-Glu, Dnp-Gln-Gly, Dnp-Leu-Gly, Ile-Gly-Phe, Lys-Pro-Asn, Pro-Arg-Gly, Pro-Asp-Gly, Pro-Gln -Tyr, Pro-Gln-Ala, Pro-Gln-Glu, Pro-Gln-Gly, Pro-Gln-His, Pro-Gln-Leu, Pro-Gln-Met, Pro-Gln-Phe, Pro-Gln-Pro, Pro-Gln-Val, Pro-Glu-Asn, Pro-Glu-Gly, Pro-His-Gly, Pro-Leu-Ala, Pro-Leu-Gly, Pro-Met-Gly, Pro-Tyr-Gly, Pro-Val-Ala, Pro-Ser-Glu-Asn, Ser-Gly-Asn, Ser-His-Ser, Ser-Ile-Pro, Thr-Glu-Lys, Tyr-Arg-Trp, Tyr-His-Ser, and Pro-Leu-MeCys.

15. A compound or salt thereof according to claim 7, wherein Y comprises a side chain comprising at least 8 non-hydrogen atoms.

16. A compound or salt thereof according to claim 15, wherein Y comprises a side chain comprising 11 non-hydrogen atoms.

17. A compound or salt thereof according to claim 16, wherein the side chain corresponds in structure to Formula (17-1):

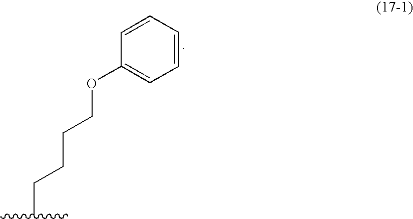

18. A compound or salt thereof according to claim 16, wherein the side chain corresponds in structure to Formula (18-1):

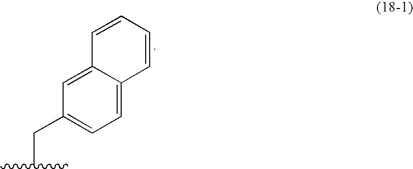

19. A compound or salt thereof according to claim 16, wherein the side chain corresponds in structure to Formula (19-1):

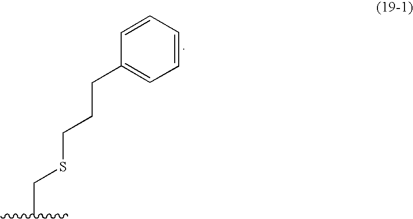

20. A compound or salt thereof according to claim 16, wherein the side chain corresponds in structure to Formula (20-1):

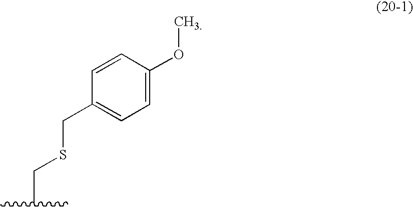

21. A compound or salt thereof according to claim 15, wherein Y comprises a side chain comprising at least 12 non-hydrogen atoms.

22. A compound or salt thereof according to claim 21, wherein the side chain corresponds in structure to Formula (22-1):

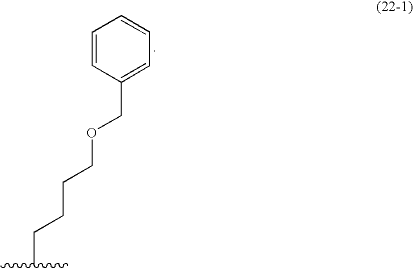

23. A compound or salt thereof according to claim 22, wherein the compound corresponds in structure to Formula (23-1):

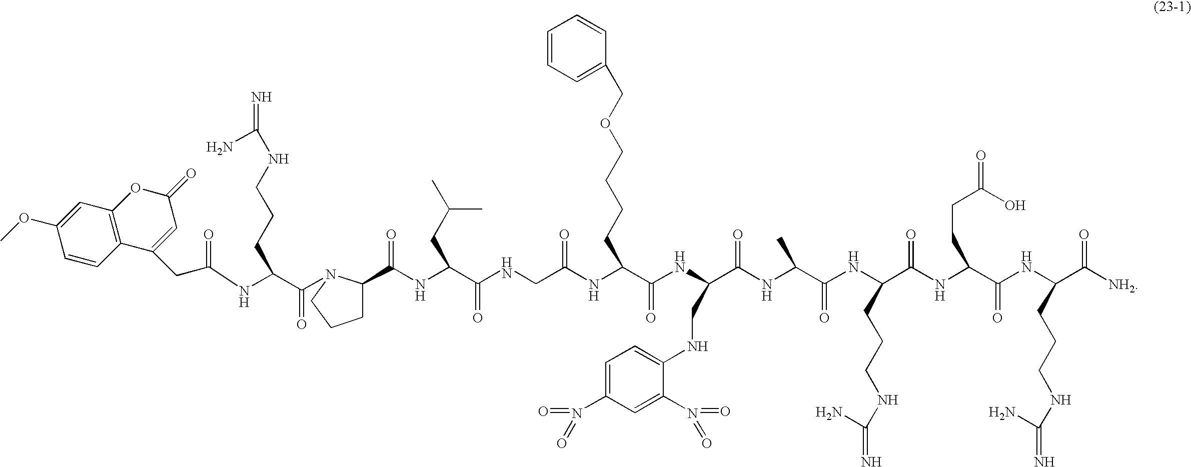

24. A compound or salt thereof according to claim 22, wherein the compound corresponds in structure to Formula (24-1):

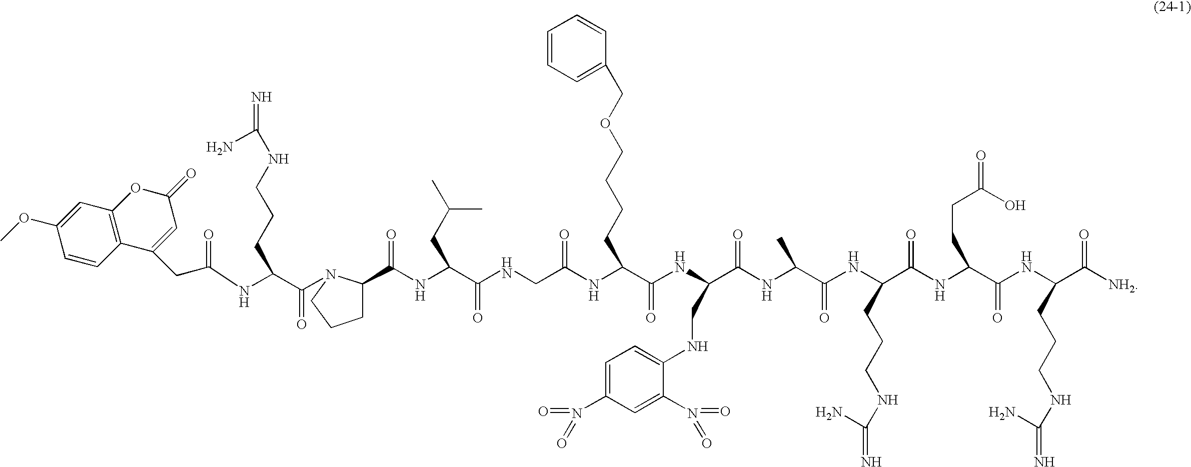

25. A compound or salt thereof according to claim 15, wherein Y comprises a side chain comprising 15 non-hydrogen atoms.

26. A compound or salt thereof according to claim 25, wherein the side chain corresponds in structure to Formula (26-1):

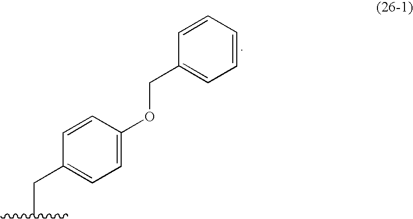

27. A compound or salt thereof according to claim 7, wherein i is zero.

28. A compound or salt thereof according to claim 7, wherein i is 1.

29. A compound or salt thereof according to claim 7, wherein j is from 3 to 5.

30. A compound or salt thereof according to claim 29, wherein i is zero or 1.

31. A compound or salt thereof according to claim 7, wherein aa(i) or aa(j) comprises an amino acid selected from the group consisting of arginine, lysine, aspartic acid, and glutamic acid.

32. A compound or salt thereof according to claim 31, wherein:
aa(i) comprises an amino acid selected from the group consisting of arginine, lysine, aspartic acid, and glutamic acid; and
aa(j) comprises an amino acid selected from the group consisting of arginine, lysine, aspartic acid, and glutamic acid.

33. A compound or salt thereof according to claim 7, wherein the compound or salt has a water solubility that is greater than a Kₘ of MMP-1, MMP-2, MMP-3, MMP-7, MMP-8, MMP-9, MMP-10, MMP-13, MMP-14, or MMP-26.

34. A compound or salt thereof according to claim 7, wherein the compound or salt has a water solubility of at least about 20 μM in 1% DMSO at pH 7.5.

35. A compound or salt thereof according to claim 34, wherein the compound or salt has a water solubility of at least about 100 μM in 1% DMSO at pH 7.5.

36. A compound or salt thereof according to claim 7, wherein the compound or salt comprises at least one amino acid having a log D contribution of less than about zero.

37. A compound or salt thereof according to claim 7, wherein the amino acid having a log D contribution of less than about zero is selected from the group consisting of D-Arginine, L-Arginine, D-Asparagine, L-Asparagine, D-Glutamic Acid, L-Glutamic acid, D-Histidine, L-Histidine, D-Glutamine, L-Glutamine, D-Lysine, L-Lysine, D-Serine, L-Serine, D-Threonine, L-Threonine, Glycine, D-Alanine, L-Alanine, D-citrulline, L-citrulline, D-ornithine, and L-ornithine.

38. A compound or salt thereof according to claim 7, wherein the fluorophore is a coumarin.

39. A compound or salt thereof according to claim 38, wherein the coumarin is substituted N-2-[(7-methoxy-2-oxo-2H-chromen-4-yl)acetyl].

40. A compound or salt thereof according to claim 38, wherein the coumarin is N-2-[(7-methoxy-2-oxo-2H-chromen-4-yl)acetyl].

41. A compound or salt thereof according to claim 7, wherein the fluorophore is a fluorescein.

42. A compound or salt thereof according to claim 41, wherein the fluorescein is substituted N-6-(4-{[3-carboxy-4-(6-hydroxy-3-oxo-3H-xanthen-9-yl)phenyl]amino}-6-chloro-1,3,5-triazin-2-yl).

43. A compound or salt thereof according to claim 41, wherein the fluorescein is N-6-(4-{[3-carboxy-4-(6-hydroxy-3-oxo-3H-xanthen-9-yl)phenyl]amino}-6-chloro-1,3,5-triazin-2-yl).

44. A compound or salt thereof according to claim 7, wherein a fluorophore is covalently attached to the peptide on one side of the X—Y bond, and a fluorescence quencher is covalently attached to the peptide on the other side of the X—Y bond.

45. A compound or salt thereof according to claim 44, wherein the fluorescence quencher is substituted (2,4-dinitrophenyl)amino.

46. A compound or salt thereof according to claim 44, wherein the fluorescence quencher is (2,4-dinitrophenyl)amino.

47. A compound or salt thereof according to claim 7, wherein a fluorophore is covalently attached to the peptide on one side of the X—Y bond, and a ligand is covalently attached to the peptide on the other side of the X—Y bond.

48. A compound or salt thereof according to claim 47, wherein the ligand is bound non-covalently to a solid support.

49. A compound or salt thereof according to claim 47, wherein the ligand is substituted biotin.

50. A compound or salt thereof according to claim 47, wherein the ligand is biotin.

51. A compound or salt thereof according to claim 47, wherein the ligand is substituted N-2-[(5-(2-oxohexahydro-1H-thieno[3,4-d]imidazol-4-yl)pentanoyl].

52. A compound or salt thereof according to claim 47, wherein the ligand is N-2-[(5-(2-oxohexahydro-1H-thieno[3,4-d]imidazol-4-yl)pentanoyl].

53. A compound or salt thereof according to claim 47, wherein the ligand is substituted epsilon amino caproic acid.

54. A compound or salt thereof according to claim 47, wherein the ligand is epsilon amino caproic acid.

55. A compound or salt thereof according to claim 7, wherein the compound or salt comprises at least one D-amino acid.

56. A compound or salt thereof according to claim 7, wherein the compound or salt comprises at least one amino acid selected from the group consisting of a beta-amino acid and a gamma-amino acid.

57. A compound or salt thereof according to claim 7, wherein Z is a protecting group.

58. A compound or salt thereof according to claim 57, wherein Z is an optionally-substituted amine.

59. A compound or salt thereof according to claim 1, wherein the compound corresponds in structure to a formula selected from group consisting of:

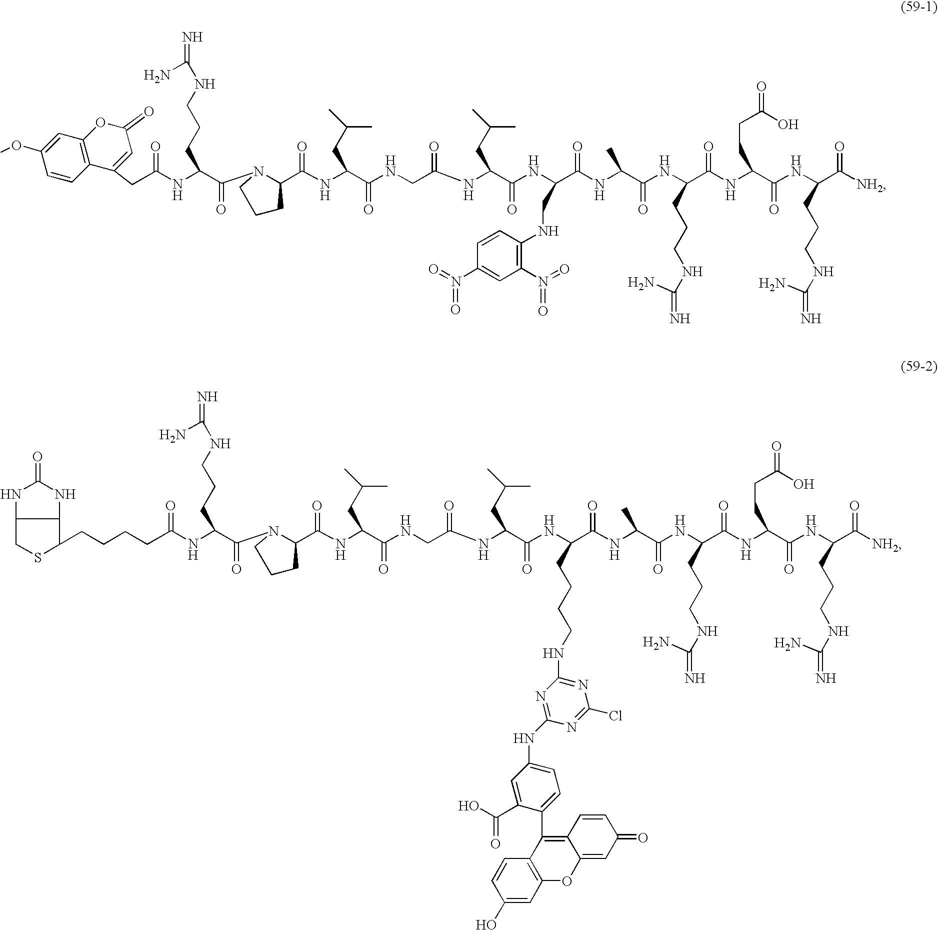

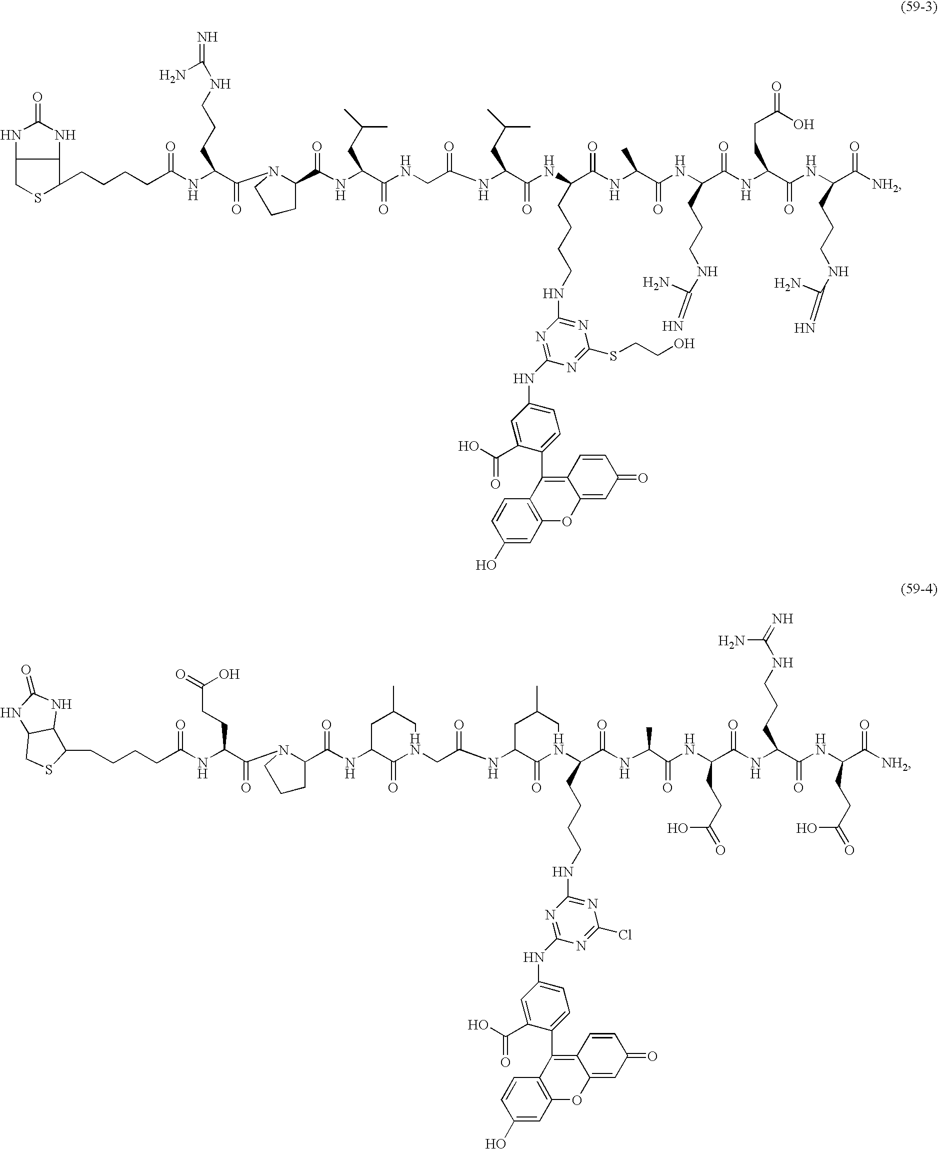

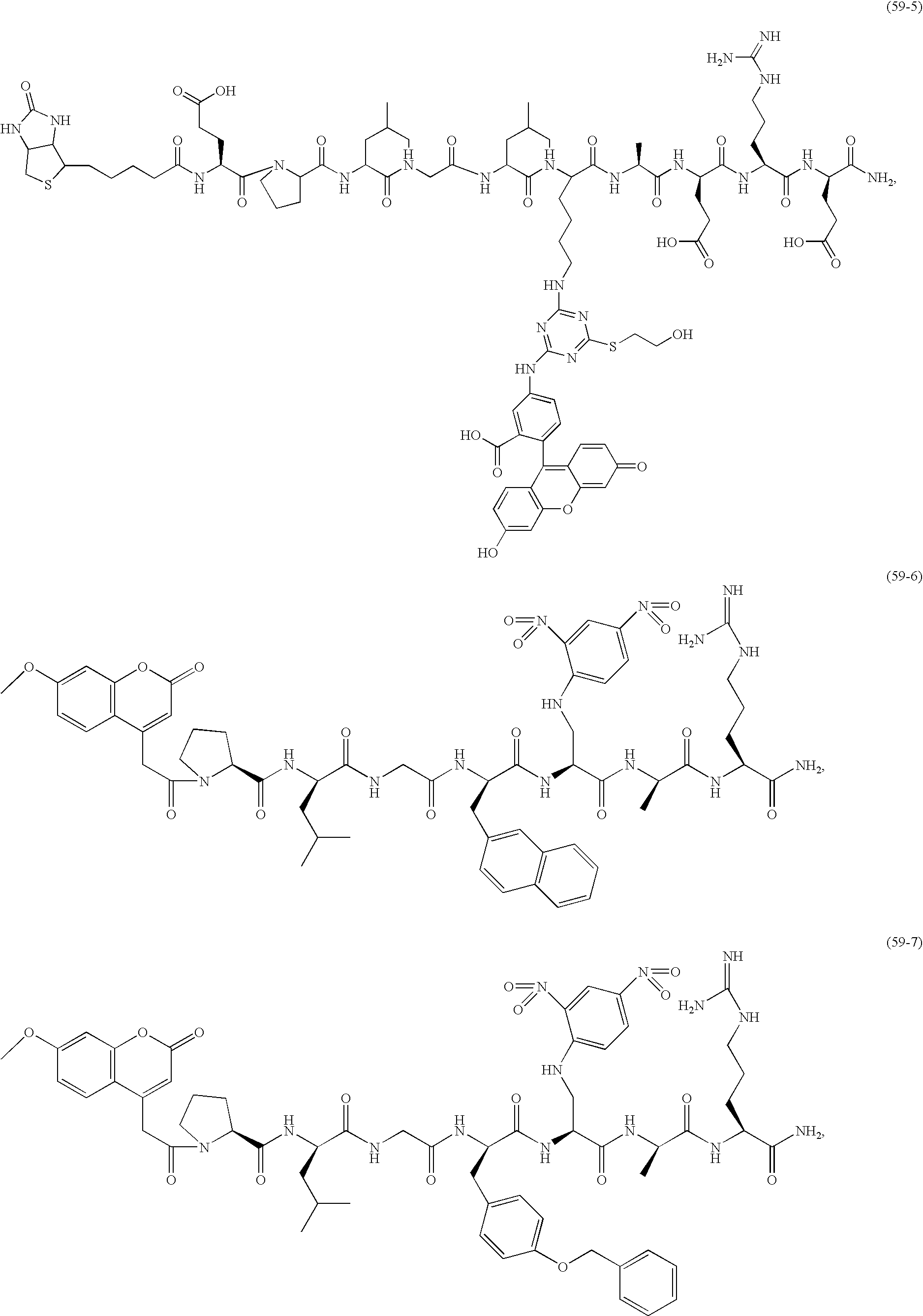

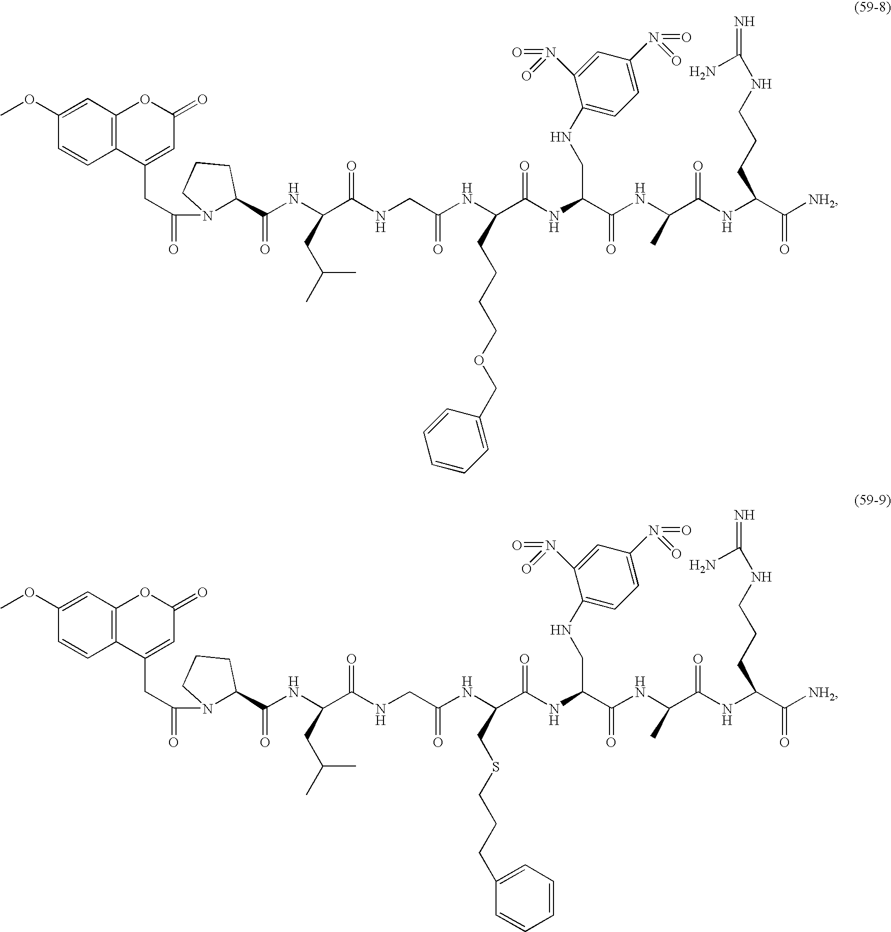

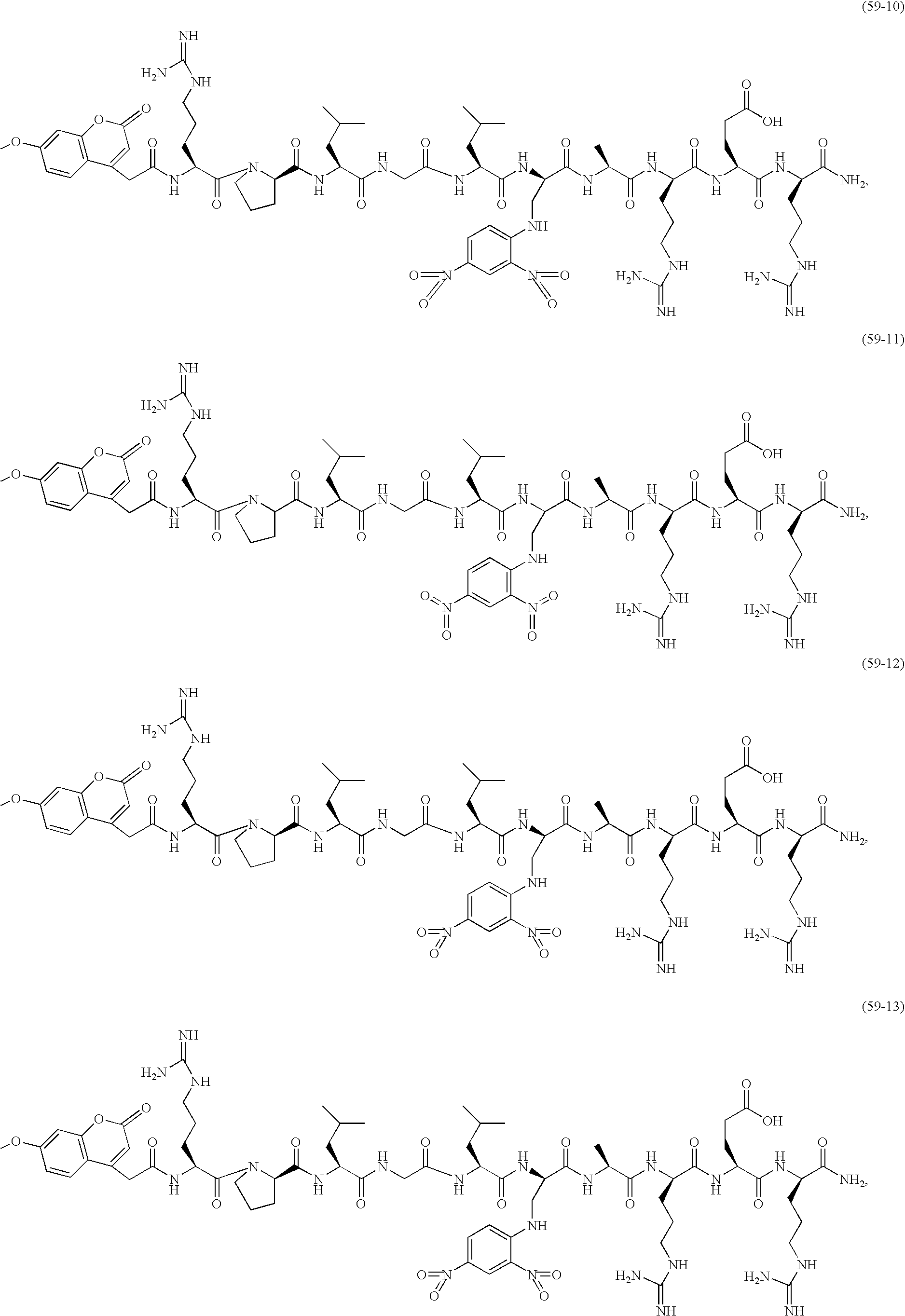

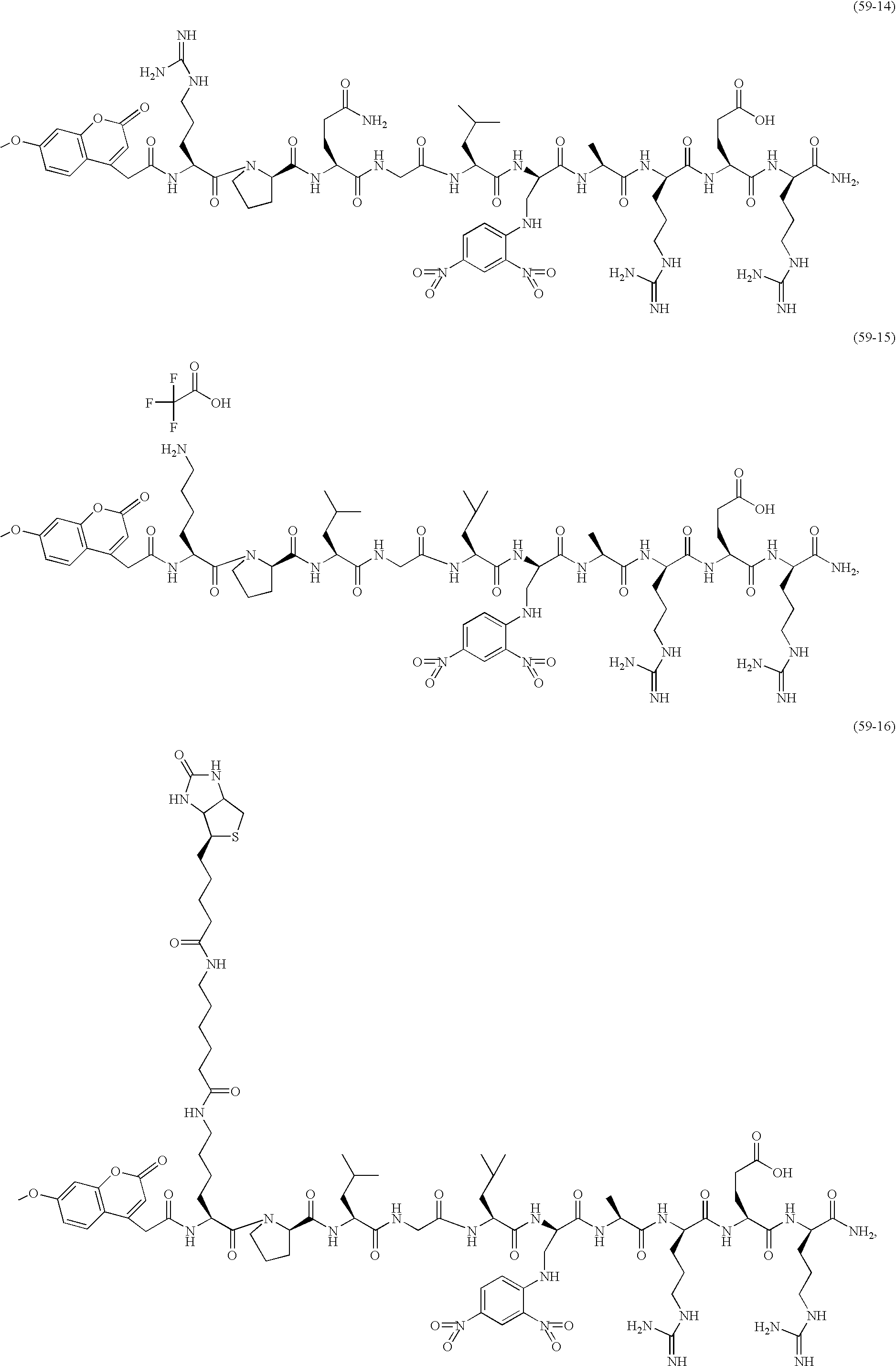

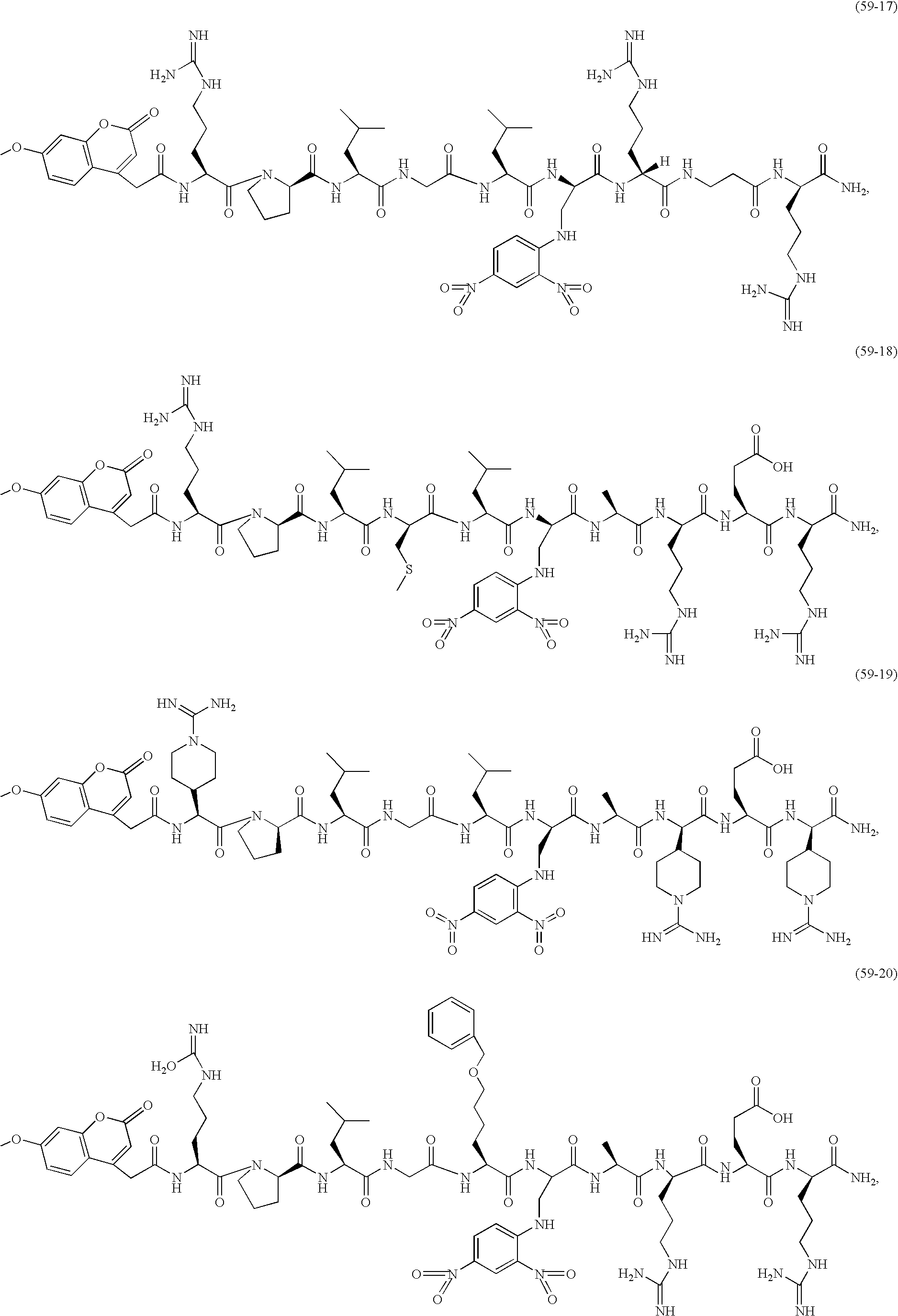

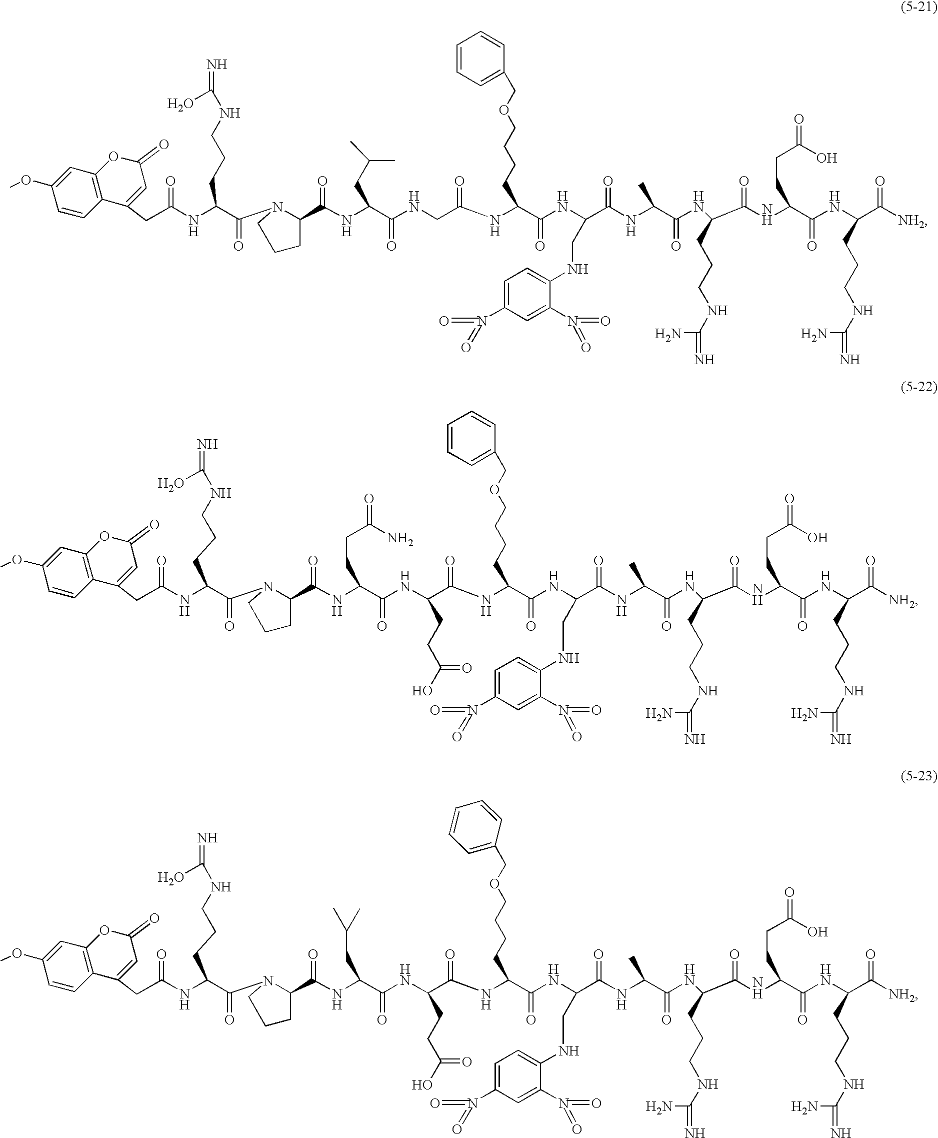

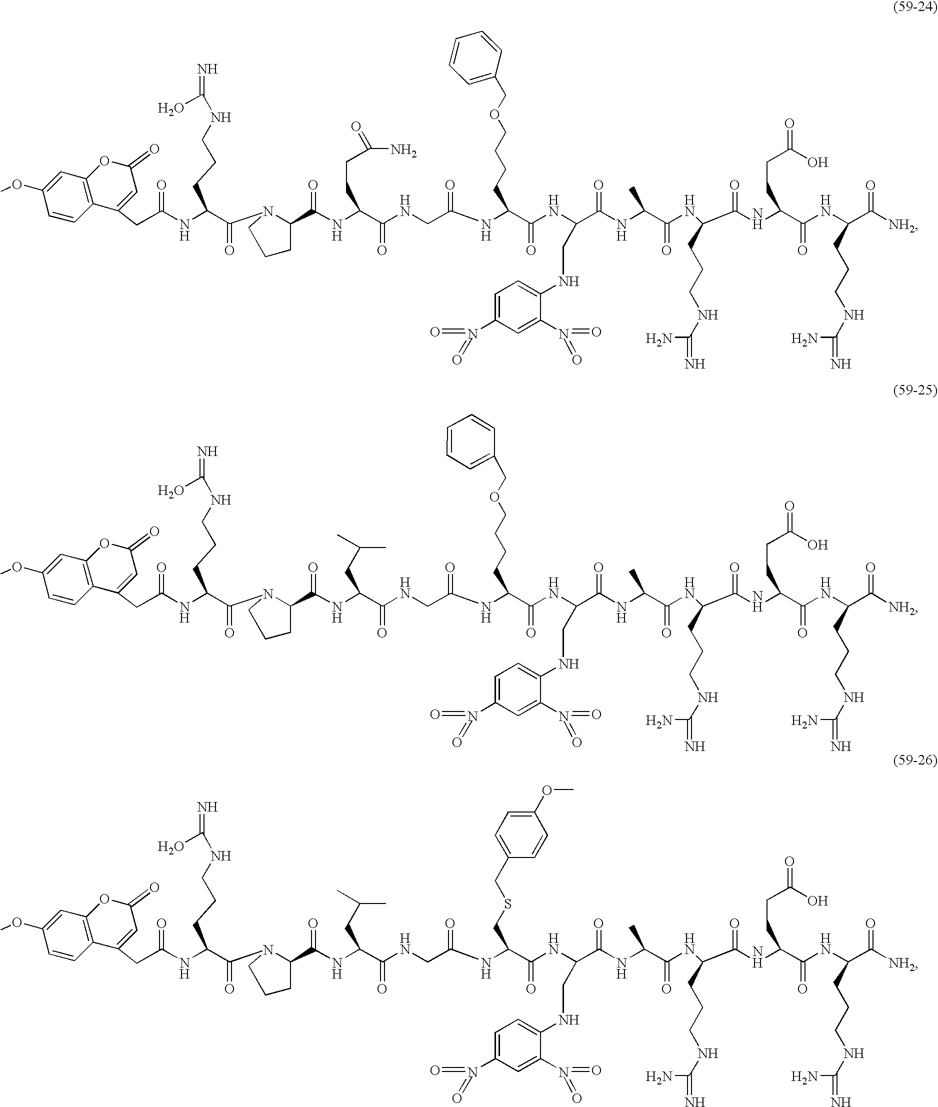

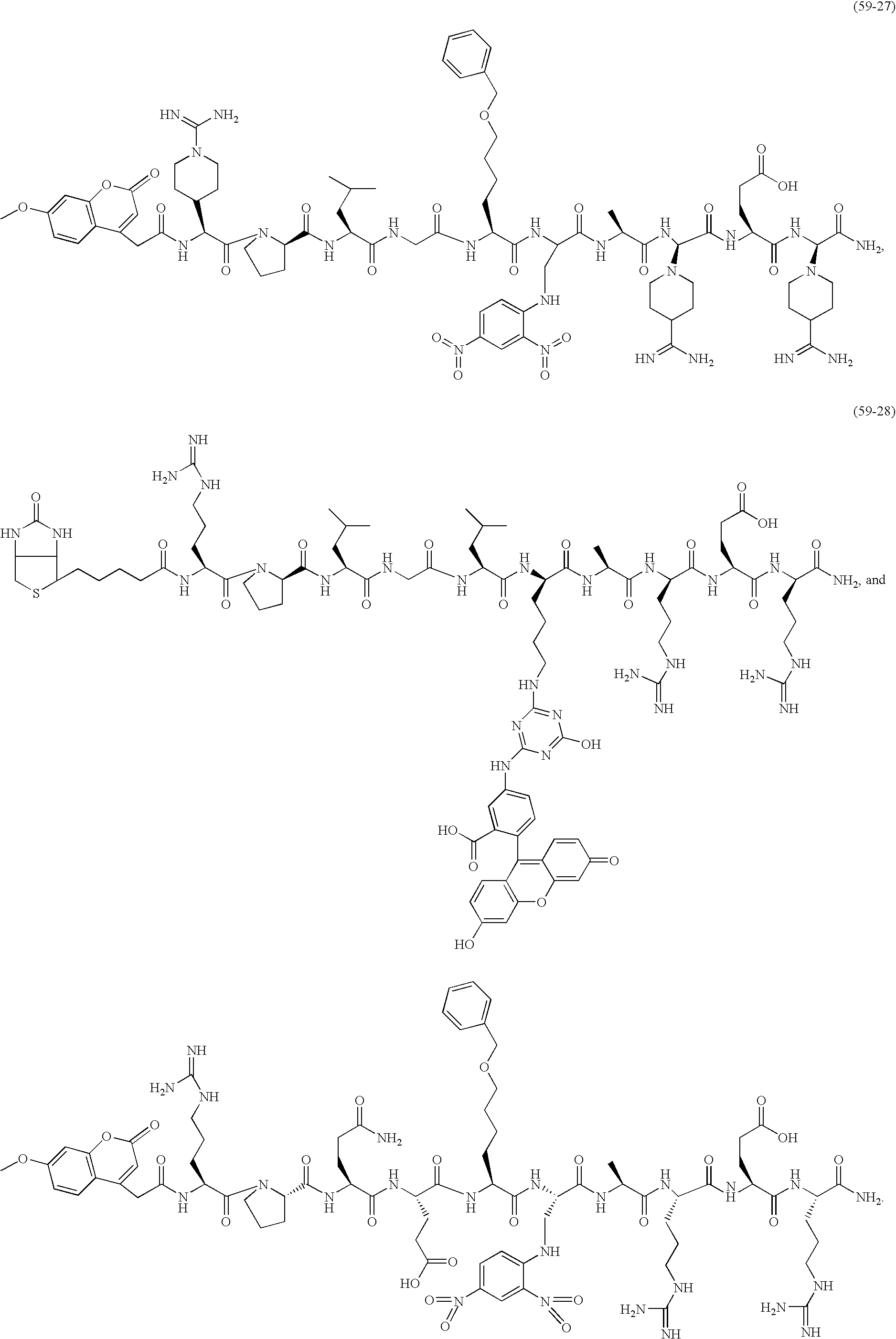

60. A compound or salt thereof according to claim 1, wherein the compound is selected from the group consisting of, SEQ ID NO: 8, SEQ ID NO: 20, SEQ ID NO: 21, SEQ ID NO: 22, SEQ ID NO: 23, SEQ ID NO: 24, and SEQ ID NO: 25.

61. A compound or a salt thereof, wherein:
the compound comprises a peptide and corresponds in structure to Formula (I):
aa₍ᵢ₎-X—Y-aa₍ⱼ₎-Z (I);
aa₍ᵢ₎ comprises a sequence of i amino acids at the N-terminus of the peptide;
aa₍ⱼ₎ comprises a sequence of j amino acids at the C-terminus of the peptide;
the peptide comprises a D-amino acid;
i is an integer from 0 to 5;
j is an integer from 1 to 6;
a fluorophore is covalently attached to the peptide on one side of the X—Y bond, and at least one of a fluorescence quencher and a ligand is covalently attached to the peptide on the other side of the X—Y bond;
X comprises an MMP recognition sequence;
Y comprises a bond or amino acid; and
Z is:
a hydroxyl group at the C-terminus of the peptide, or
a protecting group at the C-terminus of the peptide.

62. A compound or salt thereof according to claim 61, wherein the recognition sequence of X is selected from the group consisting of Ala-Gln-Gly, Ala-Met-His, Asn-Gln-Gly, Asp-Lys-Glu, Dnp-Gln-Gly, Dnp-Leu-Gly, Ile-Gly-Phe, Lys-Pro-Asn, Pro-Arg-Gly, Pro-Asp-Gly, Pro-Gln -Tyr, Pro-Gln-Ala, Pro-Gln-Gln, Pro-Gln-Glu, Pro-Gln-Gly, Pro-Gln-His, Pro-Gln-Leu, Pro-Gln-Met, Pro-Gln-Phe, Pro-Gln-Pro, Pro-Gln-Val, Pro-Glu-Asn, Pro-Glu-Gly, Pro-His-Gly, Pro-Leu-Ala, Pro-Leu-Gly, Pro-Met-Gly, Pro-Tyr-Ala, Pro-Tyr-Gly, Pro-Val-Ala, Pro-Val-Glu, Pro-Ser-Glu-Asn, Ser-Gly-Asn, Ser-His-Ser, Ser-Ile-Pro, Thr-Glu-Lys, Tyr-Arg-Trp, Tyr-His-Ser, and Pro-Leu-MeCys.

63. A compound or salt thereof according to claim 61, wherein the compound corresponds in structure to a formula selected from group consisting of:

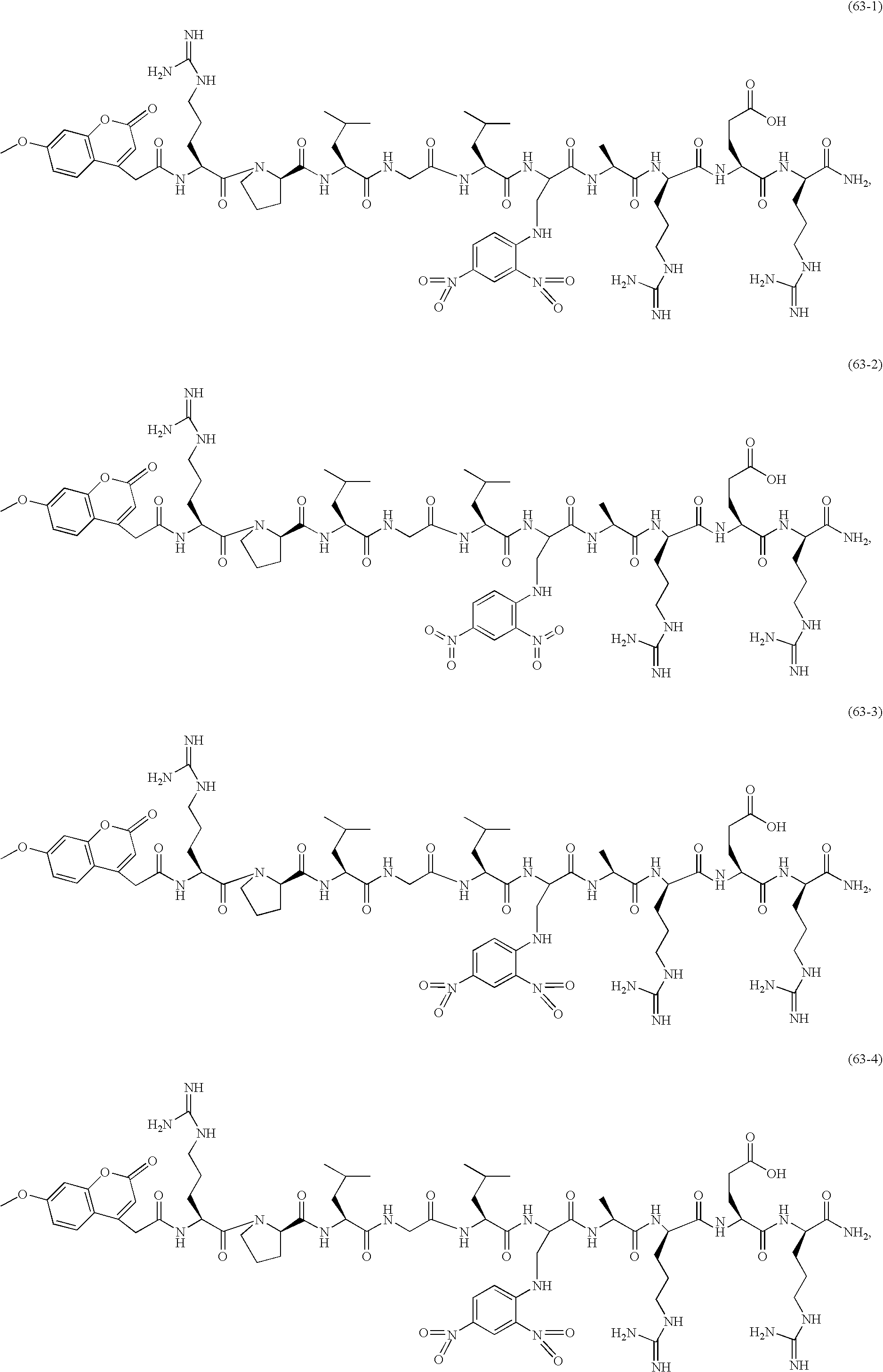

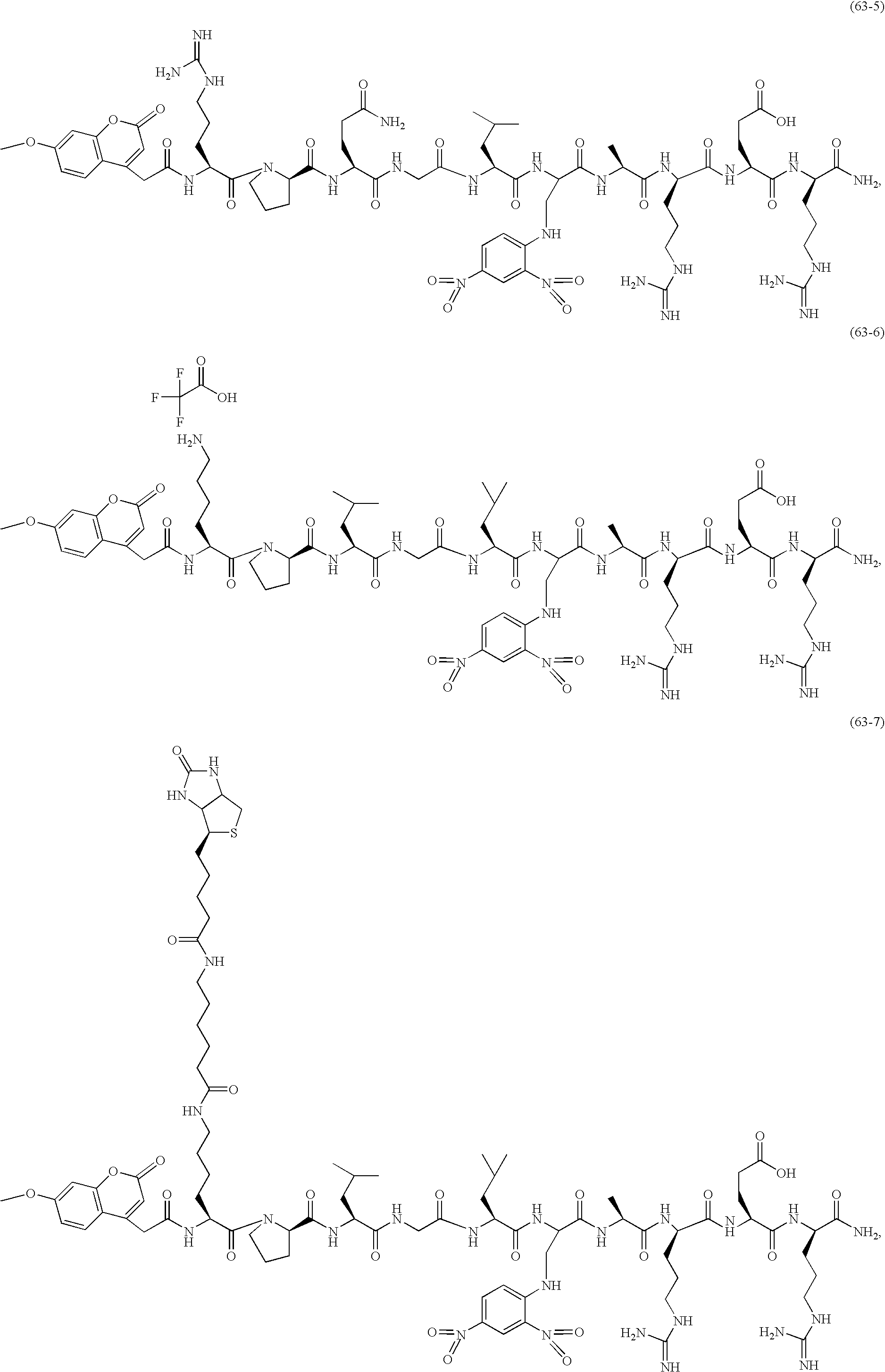

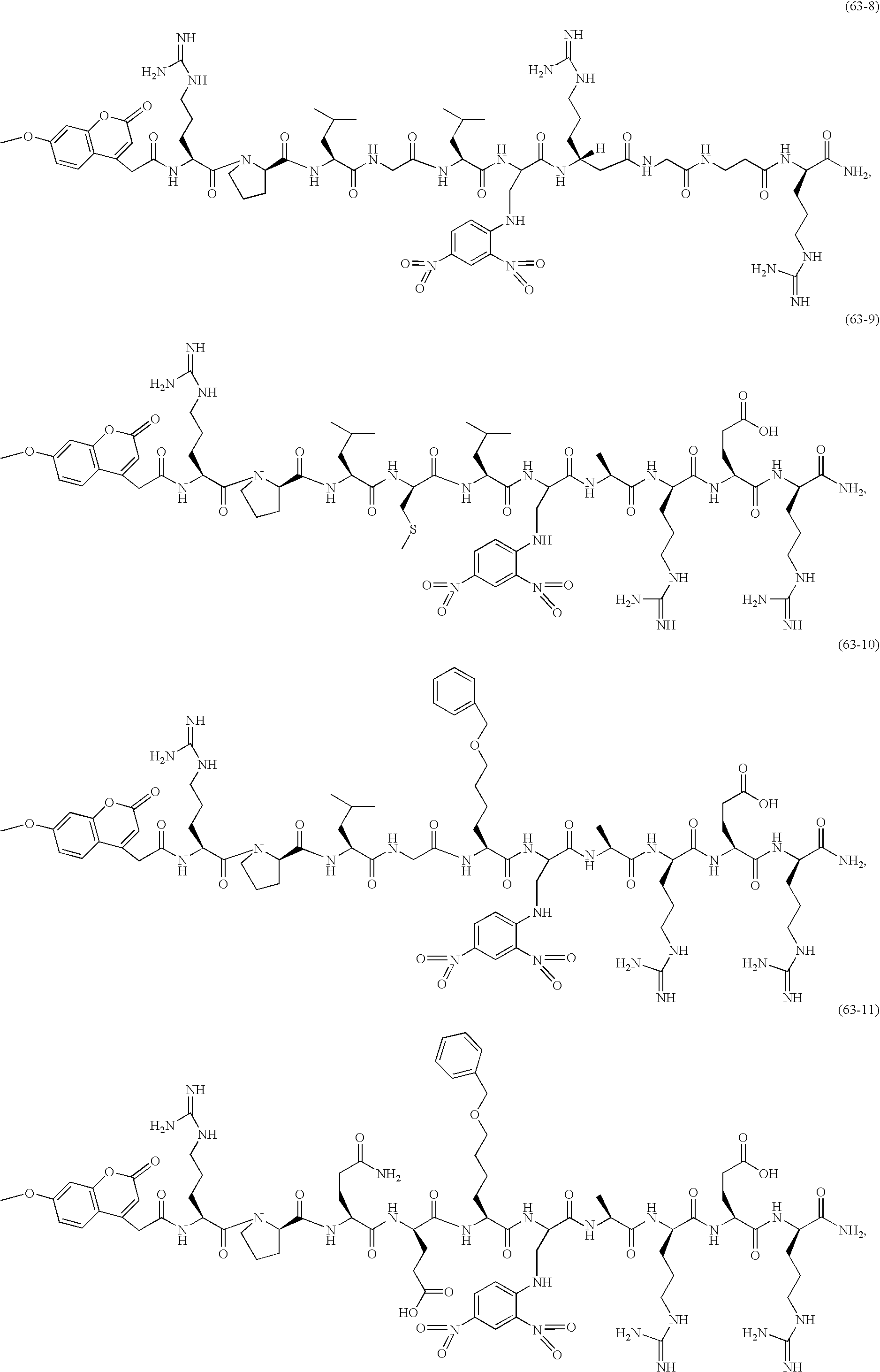

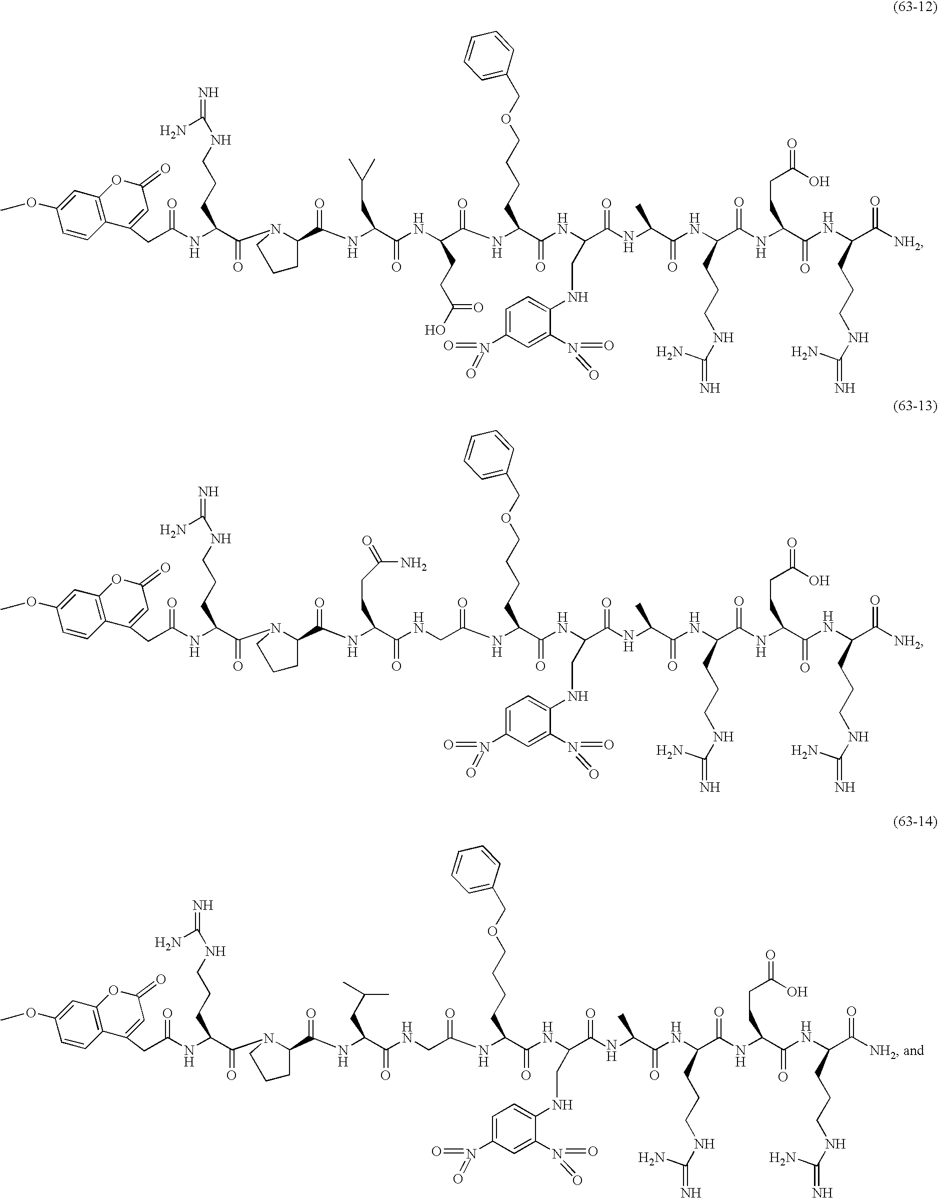

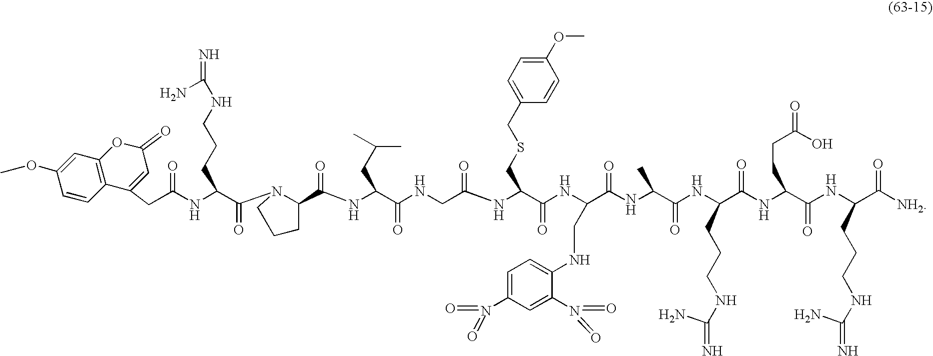

64. A compound or a salt thereof, wherein:
the compound comprises a peptide, and
the peptide comprises an amino acid selected from the group consisting of phenyloxynorleucine and benzyloxynorleucine.

65. A compound or salt thereof according to claim 64, wherein the compound or salt is characterized in that MMP-2, MMP-9, or MMP-13 reacts with the compound or salt to cleave the compound or salt at a bond between two amino acids when the compound or salt is combined with the MMP-2, MMP-9, or MMP-13.

66. A compound or salt thereof according to claim 65, wherein the compound or salt comprises phenyloxynorleucine.

67. A compound or salt thereof according to claim 66, wherein the compound or salt is characterized in that MMP-2, MMP-9, or MMP-13 reacts with the compound or salt to cleave the compound or salt at a bond between the phenyloxynorleucine and another amino acid when the compound or salt is combined with the MMP-2, MMP-9, or MMP-13.

68. A compound or salt thereof according to claim 66, wherein the compound corresponds in structure to Formula (68-1):

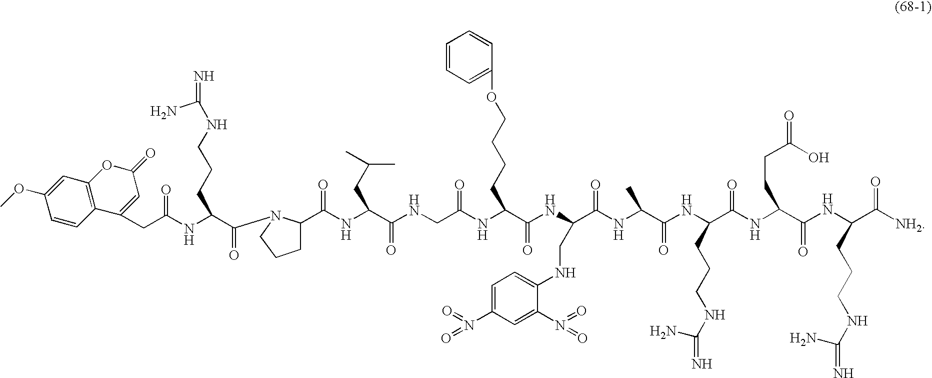

69. A compound or salt thereof according to claim 65, wherein the compound or salt comprises benzyloxynorleucine.

70. A compound or salt thereof according to claim 69, wherein the compound or salt is characterized in that MMP-2, MMP-9, or MMP-13 reacts with the compound or salt to cleave the compound or salt at a bond between the benzyloxynorleucine and another amino acid when the compound or salt is combined with the MMP-2, MMP-9, or MMP-13.

71. A compound or salt thereof according to claim 69, wherein the compound corresponds in structure to a formula selected from group consisting of:

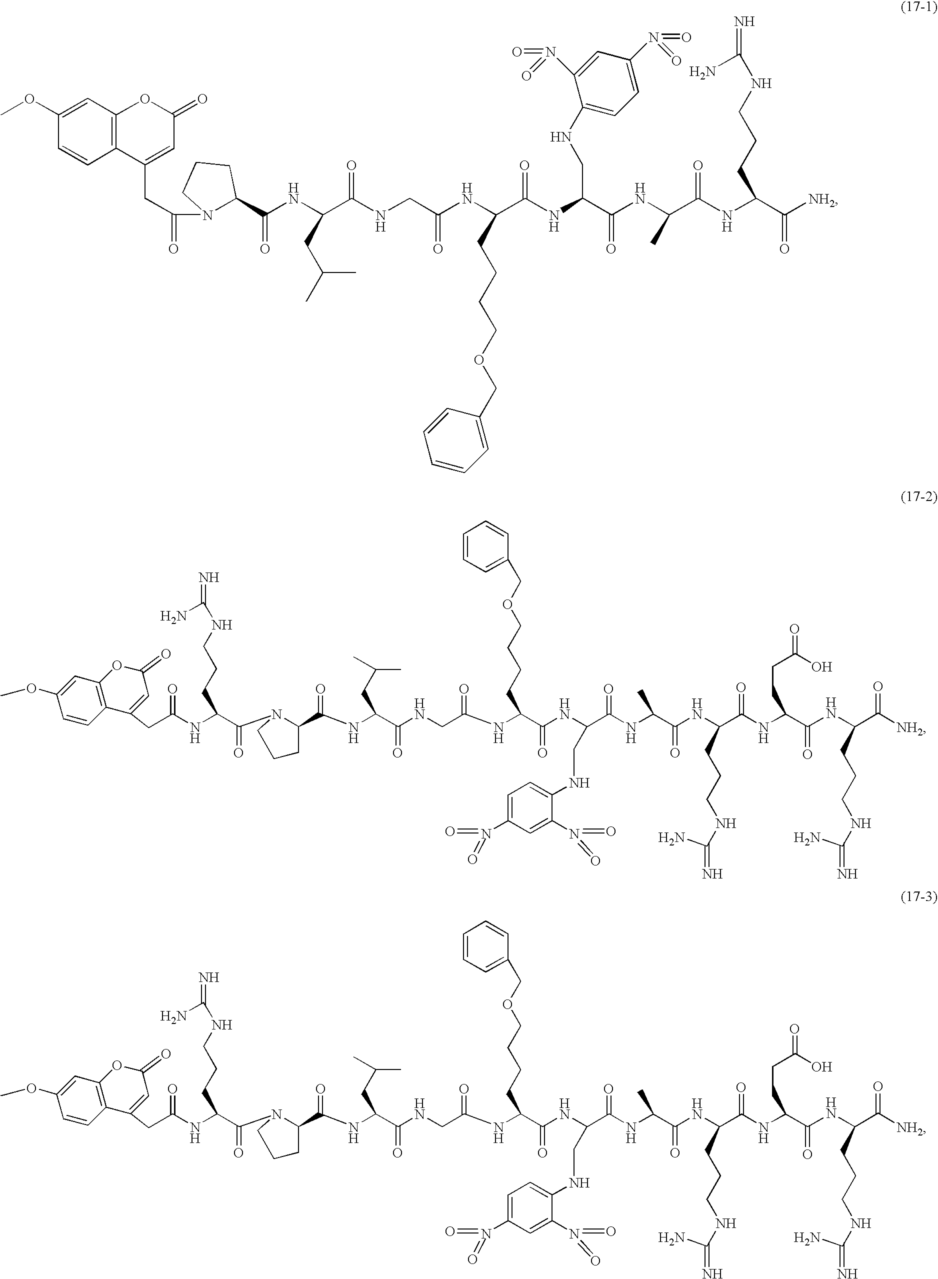

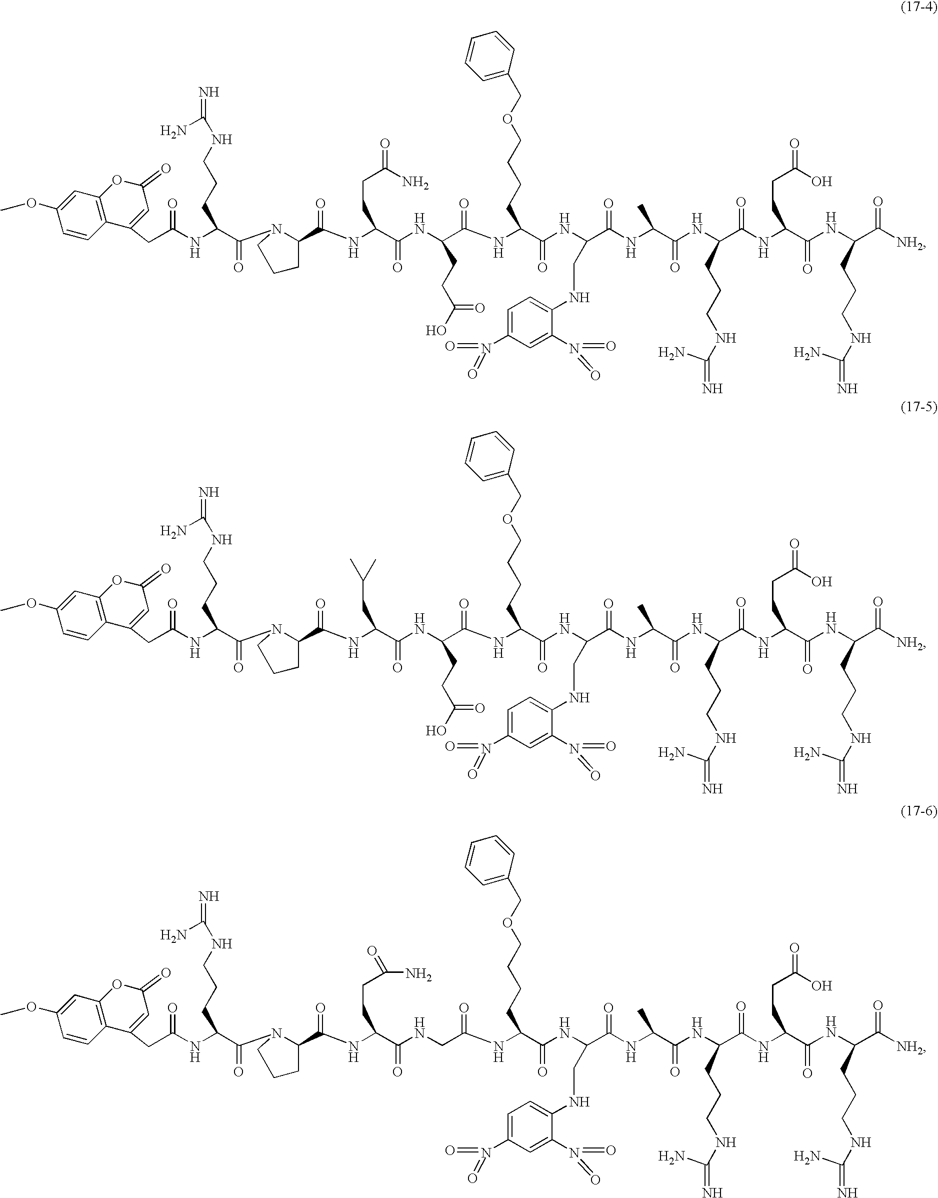

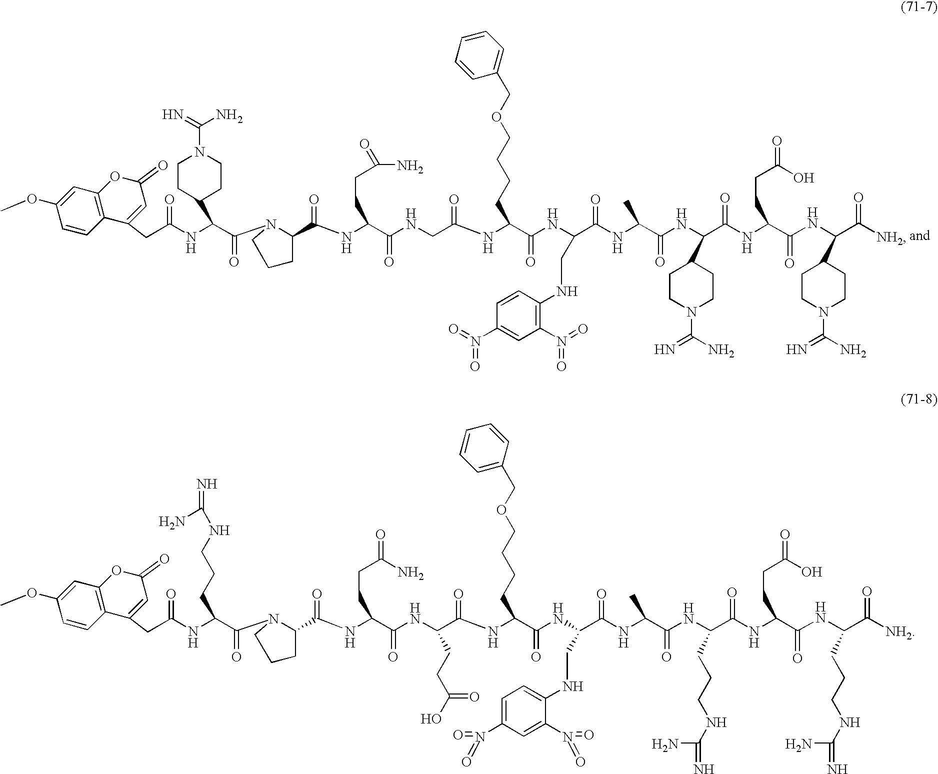

72. A method for determining the activity of a matrix metalloprotease, wherein the method comprises combining the matrix metalloprotease with a compound or salt recited in claim 1.

73. A method according to claim 72, wherein the compound or salt is a compound or salt recited in claim 59.

74. A method according to claim 72, wherein the matrix metalloprotease is MMP-2.

75. A method according to claim 72, wherein the matrix metalloprotease is MMP-9.

76. A method according to claim 72, wherein the matrix metalloprotease is MMP-13.

77. A method according to claim 72, wherein the method comprises combining:
one or more of MMP-2, MMP-9, and MMP-13;
one or more of MMP-1 and MMP-7; and
the compound or salt recited in claim 1.

78. A method for ex-vivo detection or monitoring of a disease associated with a pathological matrix metalloprotease level, wherein the method comprises measuring a cleavage of a compound or salt recited in claim 1 at the bond between X and Y.

79. A method according to claim 78, wherein the compound or salt is a compound or salt recited in claim 59.

80. A method according to claim 78, wherein the matrix metalloprotease is MMP-2.

81. A method according to claim 78, wherein the matrix metalloprotease is MMP-9.

82. A method according to claim 78, wherein the matrix metalloprotease is MMP-13.

83. A method for determining the activity of a matrix metalloprotease in a biological sample, wherein the method comprises:
combining the biological sample with a compound or salt recited in claim 1 to form a mixture, and
analyzing the mixture for the presence of a reaction product of the compound or salt with the matrix metalloprotease.

84. A method according to claim 83, wherein the compound or salt is a compound or salt recited in claim 59.

85. A method according to claim 83, wherein the matrix metalloprotease is MMP-2.

86. A method according to claim 83, wherein the matrix metalloprotease is MMP-9.

87. A method according to claim 83, wherein the matrix metalloprotease is MMP-13.

88. A method for measuring inhibitory activity of a prospective inhibitor of a matrix metalloprotease, wherein the method comprises:
combining the following to form a mixture:
a compound or salt recited in claim 1,
the prospective inhibitor, and
the matrix metalloprotease; and
analyzing the mixture for the presence of a reaction product of the compound or salt with the matrix metalloprotease.

89. A method according to claim 88, wherein the compound or salt is a compound or salt recited in claim 59.

90. A method according to claim 88, wherein the matrix metalloprotease is MMP-2.

91. A method according to claim 88, wherein the matrix metalloprotease is MMP-9.

92. A method according to claim 88, wherein the matrix metalloprotease is MMP-13.

93. A method for determining the activity of a matrix metalloprotease, wherein the method comprises combining the matrix metalloprotease with a compound or salt recited in claim 61.

94. A method according to claim 93, wherein the compound or salt is a compound or salt recited in claim 63.

95. A method according to claim 93, wherein the matrix metalloprotease is MMP-2.

96. A method according to claim 93, wherein the matrix metalloprotease is MMP-9.

97. A method according to claim 93, wherein the matrix metalloprotease is MMP-13.

98. A method according to claim 93, wherein the method comprises combining:
one or more of MMP-2, MMP-9, and MMP-13;
one or more of MMP-1 or MMP-7; and
the compound or salt recited in claim 61.

99. A method for ex-vivo detection or monitoring of a disease associated with a pathological matrix metalloprotease level, wherein the method comprises measuring a cleavage of a compound or salt recited in claim 61 at the bond between X and Y.

100. A method according to claim 99, wherein the compound or salt is a compound or salt recited in claim 63.

101. A method according to claim 99, wherein the matrix metalloprotease is MMP-2.

102. A method according to claim 99, wherein the matrix metalloprotease is MMP-9.

103. A method according to claim 99, wherein the matrix metalloprotease is MMP-13.

104. A method for determining the activity of a matrix metalloprotease in a biological sample, wherein the method comprises:
combining the biological sample with a compound or salt recited in claim 61 to form a mixture, and
analyzing the mixture for the presence of a reaction product of the compound or salt with the matrix metalloprotease.

105. A method according to claim 104, wherein the compound or salt is a compound or salt recited in claim 63.

106. A method according to claim 104, wherein the matrix metalloprotease is MMP-2.

107. A method according to claim 104, wherein the matrix metalloprotease is MMP-9.

108. A method according to claim 104, wherein the matrix metalloprotease is MMP-13.

109. A method for measuring inhibitory activity of a prospective inhibitor of a matrix metalloprotease, wherein the method comprises:
combining the following to form a mixture:
a compound or salt recited in claim 61,
the prospective inhibitor, and
the matrix metalloprotease; and
analyzing the mixture for the presence of a reaction product of the compound or salt with the matrix metalloprotease.

110. A method according to claim 109, wherein the compound or salt is a compound or salt recited in claim 63.

111. A method according to claim 109, wherein the matrix metalloprotease is MMP-2.

112. A method according to claim 109, wherein the matrix metalloprotease is MMP-9.

113. A method according to claim 109, wherein the matrix metalloprotease is MMP-13.

114. A method for determining the activity of a matrix metalloprotease, wherein the method comprises combining the matrix metalloprotease with a compound or salt recited in claim 64.

115. A method according to claim 114, wherein the compound or salt is a compound or salt recited in claim 68 or 71.

116. A method according to claim 114, wherein the matrix metalloprotease is MMP-2.

117. A method according to claim 114, wherein the matrix metalloprotease is MMP-9.

118. A method according to claim 114, wherein the matrix metalloprotease is MMP-13.

119. A method according to claim 114, wherein the method comprises combining:
one or more of MMP-2, MMP-9, and MMP-13;
one or more of MMP-1 and MMP-7; and
the compound or salt recited in claim 64.

120. A method for ex-vivo detection or monitoring of a disease associated with a pathological matrix metalloprotease level, wherein the method comprises measuring a cleavage of a compound or salt recited in claim 64.

121. A method according to claim 120, wherein the compound or salt is a compound or salt recited in claim 68 or 71.

122. A method according to claim 120, wherein the matrix metalloprotease is MMP-2.

123. A method according to claim 120, wherein the matrix metalloprotease is MMP-9.

124. A method according to claim 123, wherein the disease is an eye disease.

125. A method according to claim 124, wherein the disease is glaucoma.

126. A method according to claim 124, wherein the disease is selected from the group consisting of macular degeneration and diabetic macular edema.

127. A method according to claim 120, wherein the matrix metalloprotease is MMP-13.

128. A method for determining the activity of a matrix metalloprotease in a biological sample, wherein the method comprises:
combining the biological sample with a compound or salt recited in claim 64 to form a mixture, and
analyzing the mixture for the presence of a reaction product of the compound or salt with the matrix metalloprotease.

129. A method according to claim 128, wherein the compound or salt is a compound or salt recited in claim 68 or 71.

130. A method according to claim 128, wherein the matrix metalloprotease is MMP-2.

131. A method according to claim 128, wherein the matrix metalloprotease is MMP-9.

132. A method according to claim 131, wherein the biological sample comprises a fluid or tissue from an eye.

133. A method according to claim 128, wherein the matrix metalloprotease is MMP-13.

134. A method for measuring inhibitory activity of a prospective inhibitor of a matrix metalloprotease, wherein the method comprises:
combining the following to form a mixture:
a compound or salt recited in claim 64,
the prospective inhibitor, and
the matrix metalloprotease; and
analyzing the mixture for the presence of a reaction product of the compound or salt with the matrix metalloprotease.

135. A method according to claim 134, wherein the compound or salt is a compound or salt recited in claim 68 or 71.

136. A method according to claim 134, wherein the matrix metalloprotease is MMP-2.

137. A method according to claim 134, wherein the matrix metalloprotease is MMP-9.

138. A method according to claim 134, wherein the matrix metalloprotease is MMP-13.

139. A kit for detecting or monitoring a disease associated with pathological activity of a matrix metalloprotease, wherein the kit comprises a compound or salt recited in claim 1.

140. A kit according to claim 139, wherein the compound or salt is a compound or salt recited in claim 59.

141. A kit for detecting or monitoring a disease associated with pathological activity of a matrix metalloprotease, wherein the kit comprises a compound or salt recited in claim 61.

142. A kit according to claim 141, wherein the compound or salt is a compound or salt recited in claim 63.

143. A kit for detecting or monitoring a disease associated with pathological activity of a matrix metalloprotease, wherein the kit comprises a compound or salt recited in claim 64.

144. A kit according to claim 143, wherein the compound or salt is a compound or salt recited in claim 68 or 71.

145. A kit for evaluating the effectiveness of a prospective inhibitor of a matrix metalloprotease, wherein the kit comprises a compound or salt recited in claim 1.

146. A kit for evaluating the effectiveness of a prospective inhibitor of a matrix metalloprotease, wherein the kit comprises a compound or salt recited in claim 61.

147. A kit for evaluating the effectiveness of a prospective inhibitor of a matrix metalloprotease, wherein the kit comprises a compound or salt recited in claim 64.

148. A compound or a salt thereof, wherein:
the compound corresponds in structure to Formula (II): 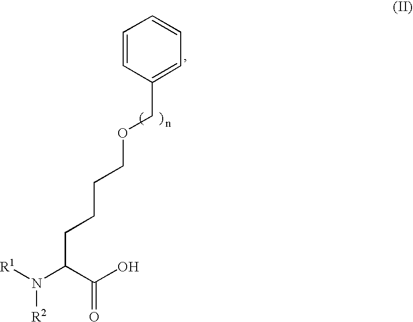
n is zero or 1, and
R¹ and R² are independently selected from the group consisting of hydrogen and a nitrogen protecting group.

149. A compound or salt thereof according to claim 148, wherein the compound corresponds in structure to Formula (IIA):

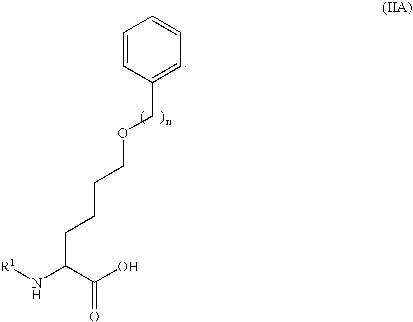

150. A compound or salt thereof according to claim 149, wherein the compound corresponds in structure to a formula selected from the group consisting of: [chemical structure]
